(19) United States
(12) Patent Application Publication
(10) Pub. No.: US 2026/0283532 A1
(43) Pub. Date: Sep. 24, 2026

(54) MEDICAL DEVICE AND METHOD FOR DETECTING ARRHYTHMIA

(71) Applicant: Medtronic, Inc., Minneapolis, MN (US)

(72) Inventors: Alfonso ARANDA HERNANDEZ, Minneapolis, MN (US); Timothy A. EBELING, Circle Pines, MN (US); Saul E. GREENHUT, Denver, CO (US); Scott R. HAWKINSON, Andover, MN (US); Troy E. JACKSON, Rogers, MN (US); Yuanzhen LIU, Palo Alto, CA (US); Irving J. SANCHEZ, Blaine, MN (US); James A. VANDER HEYDEN, Delano, MN (US); Xusheng ZHANG, Shoreview, MN (US)

(21) Appl. No.: 19/469,882

(22) PCT Filed: Feb. 29, 2024
(86) PCT No.: PCT/IB2024/051969

§ 371 Date: Sep. 26, 2025

Related U.S. Application Data
(60) Provisional application No. 63/492,474, filed on Mar. 27, 2023.

(51) Int. Cl. A61B 5/363; A61N 1/362; A61N 1/39

(52) U.S. Cl. CPC A61B 5/363; A61N 1/3621; A61N 1/39622; A61N 1/3987

(57) ABSTRACT

A medical device is configured to obtain a cardiac signal segment from a cardiac signal sensed by a sensing circuit of the medical device and perform a morphology analysis of the cardiac signal segment for classifying the cardiac signal segment as being one of ventricular tachyarrhythmia, non-ventricular tachyarrhythmia or asystole. The medical device may override a rejection rule for withholding a tachyarrhythmia detection by detecting ventricular tachyarrhythmia in response to a required number of tachyarrhythmia intervals being detected and the cardiac signal segment being classified as ventricular tachyarrhythmia based on the morphology analysis when the rejection rule is met.

This application claims the benefit of U.S. Provisional Patent Application Ser. No. 63/492,474, filed Mar. 27, 2023, the entire content of which is incorporated herein by reference.

### TECHNICAL FIELD

The disclosure relates generally to a medical device and method for sensing cardiac signals and detecting arrhythmia.

### BACKGROUND

Medical devices may sense electrophysiological signals from the heart, brain, nerve, muscle or other tissue. Such devices may be implantable, wearable or external devices using implantable and/or surface (skin) electrodes for sensing the electrophysiological signals. In some cases, such devices may be configured to deliver a therapy based on the sensed electrophysiological signals. For example, implantable or external cardiac pacemakers, cardioverter defibrillators, cardiac monitors and the like, sense cardiac electrical signals from a patient's heart. The medical device may sense cardiac electrical signals from a heart chamber and deliver electrical stimulation therapies to the heart chamber using electrodes carried by a transvenous medical electrical lead that positions electrodes within the patient's heart.

A cardiac pacemaker or cardioverter defibrillator may deliver therapeutic electrical stimulation to the heart via electrodes carried by one or more medical electrical leads coupled to the medical device. The electrical stimulation may include electrical pulses such as pacing pulses and/or cardioversion or defibrillation shocks. In some cases, a medical device may sense cardiac electrical signals attendant to the intrinsic depolarizations of the myocardium and control delivery of stimulation pulses to the heart based on sensed cardiac electrical signals. Cardiac signals sensed within a heart chamber using endocardial electrodes carried by transvenous leads, for example, generally have a high signal strength and quality for reliably sensing cardiac electrical events, such as ventricular R-waves sensed from within a ventricle. Upon detection of an abnormal rhythm, such as bradycardia, tachycardia or fibrillation, an appropriate electrical stimulation pulse or pulses may be delivered to restore or maintain a more normal rhythm of the heart. For example, an implantable cardioverter defibrillator (ICD) may deliver pacing pulses to the heart of the patient upon detecting bradycardia or tachycardia or deliver cardioversion or defibrillation (CV/DF) shocks to the heart upon detecting tachycardia or fibrillation.

### SUMMARY

In general, this disclosure is directed to a medical device and techniques for sensing cardiac electrical signals, detecting arrhythmias and delivering cardiac electrical stimulation therapies as needed. In some examples, the medical device may be coupled to an extracardiac medical lead carrying electrodes positioned outside of the heart for sensing cardiac electrical signals and delivering electrical stimulation pulses, including pacing pulses and/or CV/DF shocks. A medical device operating according to the techniques disclosed herein is configured to perform a gross morphology analysis (GMA) of a cardiac signal segment for use in detecting ventricular tachyarrhythmia and/or redetecting ventricular tachyarrhythmia before and/or after an electrical stimulation therapy has been delivered, e.g., a CV/DF shock. The cardiac signal segment can have a starting time that is independent of the timing of a sensed ventricular event signal.

The cardiac signal segment may be classified as being a tachyarrhythmia segment, a non-tachyarrhythmia segment or an asystole segment based on the GMA. When the classification of the cardiac signal segment is not a tachyarrhythmia segment (e.g., a non-tachyarrhythmia segment or asystole segment), the medical device may withhold a ventricular tachyarrhythmia detection based on a rejection rule being met. The medical device may be configured to perform cardiac signal analysis for determining when a rejection rule is met for withholding a ventricular tachyarrhythmia detection based on evidence of oversensing or evidence of supraventricular tachyarrhythmia (SVT). When a cardiac signal segment is classified as a tachyarrhythmia segment based on the GMA, control circuitry of the medical device may override a rejection rule for withholding a ventricular tachyarrhythmia detection. The medical device may detect ventricular tachyarrhythmia when a required number of tachyarrhythmia intervals are detected based on sensed ventricular event signals and a rejection rule is not met or is overridden due to a cardiac signal segment being classified as tachyarrhythmia based on the GMA.

In one example, the disclosure provides a medical device including a sensing circuit configured to sense a plurality of cardiac signals and sense ventricular event signals from the cardiac signals. The medical device includes a control circuit in communication with the sensing circuit. The control circuit may be configured to, for each of a plurality of ventricular event signals sensed by the sensing circuit, determine sensed event data from the cardiac signals. The control circuit is further configured to obtain a cardiac signal segment from the plurality of cardiac signals sensed by the sensing circuit. The cardiac signal segment can have a starting time that is independent of a timing of a ventricular event signal sensed by the sensing circuit. The control circuit may perform a morphology analysis of the cardiac signal segment for classifying the cardiac signal segment as being one of ventricular tachyarrhythmia, non-ventricular tachyarrhythmia or asystole. The control circuit may determine from the ventricular event signals sensed by the sensing circuit that a required number of tachyarrhythmia intervals is reached for detecting ventricular tachyarrhythmia. The control circuit may determine that a rejection rule for withholding a tachyarrhythmia detection is met based on the sensed event data. The control circuit may determine that the cardiac signal segment is classified as ventricular tachyarrhythmia based on the morphology analysis. The control circuit may override the rejection rule for withholding a tachyarrhythmia detection by detecting ventricular tachyarrhythmia in response to the required number of tachyarrhythmia intervals being reached and the cardiac signal segment being classified as ventricular tachyarrhythmia based on the morphology analysis. The medical device may further include a therapy delivery circuit including a capacitor. The therapy delivery circuit can be configured to start charging the capacitor for delivering a therapy in response to the control circuit detecting the ventricular tachyarrhythmia.

In another example, the disclosure provides a method including sensing a plurality of cardiac signals and sensing ventricular event signals from the plurality of cardiac signals. The method may include, for each of a plurality of ventricular event signals sensed by the sensing circuit, determining sensed event data from the plurality of cardiac signals. The method further includes obtaining a cardiac signal segment from the plurality of cardiac signals. The cardiac signal segment can have a starting time that is independent of a timing of a ventricular event signal sensed by the sensing circuit. The method may further include performing a morphology analysis of the cardiac signal segment for classifying the cardiac signal segment as being one of ventricular tachyarrhythmia, non-ventricular tachyarrhythmia or asystole. The method may include determining from the ventricular event signals that a required number of tachyarrhythmia intervals is reached for detecting ventricular tachyarrhythmia. The method may include determining that a rejection rule for withholding a tachyarrhythmia detection is met based on the sensed event data. The method may include determining that the cardiac signal segment is classified as ventricular tachyarrhythmia based on the morphology analysis. The method may include overriding the rejection rule for withholding a tachyarrhythmia detection by detecting ventricular tachyarrhythmia in response to the required number of tachyarrhythmia intervals being reached and the cardiac signal segment being classified as ventricular tachyarrhythmia based on the morphology analysis. The method may include starting charging a capacitor for delivering a therapy in response to detecting the ventricular tachyarrhythmia.

In yet another example, the disclosure provides a non-transitory computer readable medium storing a set of instructions that, when executed by a control circuit of a medical device, cause the medical device to sense a plurality of cardiac signals and sense ventricular event signals from the plurality of cardiac signals. The instructions may further cause the medical device to, for each of a plurality of ventricular event signals sensed by the sensing circuit, determine sensed event data from the plurality of cardiac signals. The instructions may further cause the medical device to obtain a cardiac signal segment from the plurality of cardiac signals. The cardiac signal segment can have a starting time that is independent of a timing of a ventricular event signal sensed by the sensing circuit. The instructions may further cause the medical device to perform a morphology analysis of the cardiac signal segment for classifying the cardiac signal segment as being one of ventricular tachyarrhythmia, non-ventricular tachyarrhythmia or asystole. The instructions may further cause the medical device to determine from the ventricular event signals that a required number of tachyarrhythmia intervals is reached for detecting ventricular tachyarrhythmia. The instructions may further cause the medical device to determine that a rejection rule for withholding a tachyarrhythmia detection is met based on the sensed event data. The instructions may further cause the medical device to determine that the cardiac signal segment is classified as ventricular tachyarrhythmia based on the morphology analysis. The instructions may further cause the medical device to override the rejection rule for withholding a tachyarrhythmia detection by detecting ventricular tachyarrhythmia in response to the required number of tachyarrhythmia intervals being reached and the first cardiac signal segment being classified as ventricular tachyarrhythmia based on the morphology analysis. The instructions may further cause the medical device to start charging a capacitor for delivering a therapy in response to detecting the ventricular tachyarrhythmia.

In another example the disclosure provides a medical device including a sensing circuit configured to sense a plurality of cardiac signals and sense ventricular event signals from at least one of the plurality of cardiac signals. The medical device includes a control circuit in communication with the sensing circuit. The control circuit may be configured to detect a ventricular tachyarrhythmia based on the plurality of cardiac signals. Subsequent to detecting the ventricular tachyarrhythmia, the control circuit may determine from the ventricular event signals sensed by the sensing circuit that a redetection number of tachyarrhythmia intervals is reached and determine when a rejection rule for withholding a tachyarrhythmia detection is met based on an analysis of the plurality of cardiac signals. The control circuit may be further configured to withhold redetection of a ventricular tachyarrhythmia when the rejection rule is met and the redetection number of tachyarrhythmia intervals is reached. The control circuit may be further configured to redetect the ventricular tachyarrhythmia when the first rejection rule is not met and the redetection number of tachyarrhythmia intervals is reached.

In another example, the disclosure provides a method including sensing a plurality of cardiac signals, sensing ventricular event signals from at least one of the plurality of cardiac signals. The method may include detecting a ventricular tachyarrhythmia based on the plurality of cardiac signals. Subsequent to detecting the ventricular tachyarrhythmia, the method may further include determining from the sensed ventricular event signals that a redetection number of tachyarrhythmia intervals is reached. The method may further include determining when a rejection rule for withholding a tachyarrhythmia detection is met based on an analysis of the plurality of cardiac signals. The method may further include withholding redetection of a ventricular tachyarrhythmia when the first rejection rule is met and the redetection number of tachyarrhythmia intervals is reached. The method may further include redetecting the ventricular tachyarrhythmia when the first rejection rule is not met and the redetection number of tachyarrhythmia intervals is reached.

In yet another example, the disclosure provides a non-transitory, computer readable medium storing a set of instructions that, when executed by a control circuit of a medical device, cause the medical device to sense a plurality of cardiac signals, sense ventricular event signals from at least one of the plurality of cardiac signals and detect a ventricular tachyarrhythmia based on the plurality of cardiac signals. The instructions may further cause the medical device to, subsequent to detecting the ventricular tachyarrhythmia, determine from the sensed ventricular event signals that a redetection number of tachyarrhythmia intervals is reached. The instructions may further cause the medical device to determine when a rejection rule for withholding a tachyarrhythmia detection is met based on an analysis of the plurality of cardiac signals. The instructions may further cause the medical device to withhold redetection of a ventricular tachyarrhythmia when the first rejection rule is met and the redetection number of tachyarrhythmia intervals is reached. The instructions may further cause the medical device to redetect the ventricular tachyarrhythmia when the first rejection rule is not met and the redetection number of tachyarrhythmia intervals is reached.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIGS. 1A and 1B are conceptual diagrams of one example of an ICD system that may be configured to sense cardiac event signals, detect arrhythmia and deliver electrical stimulation therapy according to the techniques disclosed herein.

FIGS. 2A-2C are conceptual diagrams of a patient implanted with an ICD system in a different implant configuration than the arrangement shown in FIGS. 1A-1B.

FIG. 3 is a conceptual diagram of an ICD according to one example.

FIG. 4 is a conceptual diagram of circuitry that may be included in a sensing circuit of the ICD shown in FIG. 3 according to one example.

FIG. 5 is a conceptual diagram of tachyarrhythmia operating states of an ICD for sensing and analyzing cardiac signals for detecting ventricular tachyarrhythmia and delivering therapy in response to a ventricular tachyarrhythmia detection according to some examples.

FIG. 6 is a conceptual diagram of operations that may be performed by an ICD during the unconcerned sensing state 1 of the tachyarrhythmia operating states shown in FIG. 5.

FIG. 7 is a flow chart of a method that may be performed by an ICD for selecting a reliable sensing channel for detecting ventricular tachyarrhythmia according to some examples.

FIG. 8 is a flow chart of a method that may be performed by an ICD for detecting suspected ventricular tachyarrhythmia undersensing during the unconcerned sensing state 1 of FIG. 6.

FIG. 9 is a flow chart of a method for performing a gross morphology analysis that of a cardiac signal segment that is acquired by an ICD independent of the timing of sensed ventricular event signals according to some examples.

FIG. 10 is a flow chart of a method for detecting ventricular tachyarrhythmia that may be performed, according to some examples, by an ICD 14 operating during the concerned state 2 of the tachyarrhythmia operating states described in conjunction with FIG. 5.

FIG. 11 is a flow chart of a method that may be performed by an ICD for determining when a cardiac event oversensing rejection rule for is met for withholding a ventricular tachyarrhythmia detection according to some examples.

FIG. 12 is a flow chart of a method that may be performed by an ICD for determining when an SVT rejection rule is met for withholding a ventricular tachyarrhythmia detection according to some examples.

FIG. 13 is a flow chart of a method that may be performed by an ICD for redetecting ventricular tachyarrhythmia while operating in the redetection state 5 of the tachyarrhythmia operating states shown in FIG. 5 according to some examples.

FIG. 14 is a diagram of a method for performing a gross morphology analysis of cardiac signal segments during a pacing period according to some examples.

### DETAILED DESCRIPTION

In general, this disclosure describes a medical device and techniques for sensing ventricular event signals, e.g., R-waves, and detecting arrhythmia. In various examples, the medical device performing the techniques disclosed herein may be included in an ICD system capable of sensing cardiac electrical signals, detecting arrhythmia based on an analysis of the sensed cardiac electrical signals, and delivering electrical stimulation therapy for treating the arrhythmia. In some examples, the ICD is coupled to an extra-cardiovascular lead. As used herein, the term “extra-cardiovascular” refers to a position outside the blood vessels, heart, and pericardium surrounding the heart of a patient. Implantable electrodes carried by extra-cardiovascular leads may be positioned extra-thoracically (outside the ribcage and sternum) or intra-thoracically (beneath the ribcage or sternum) but generally not in intimate contact with myocardial tissue, e.g., within the heart or within the pericardium. In other examples, a transvenous extra-cardiac lead may carry implantable electrodes that can be positioned intravenously but outside the heart in an extra-cardiac location, e.g., within the internal thoracic vein, jugular vein, or other vein, for sensing cardiac electrical signals and delivering cardiac pacing pulses.

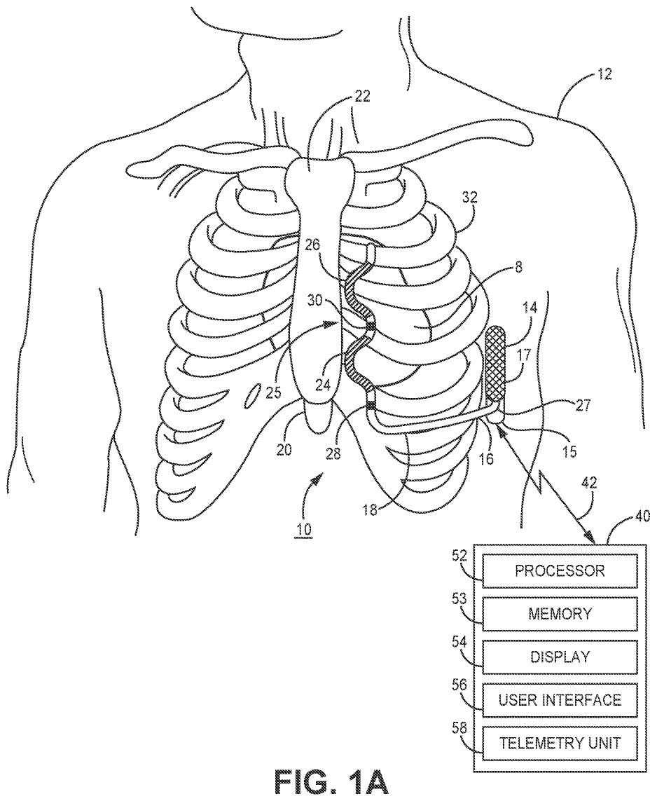

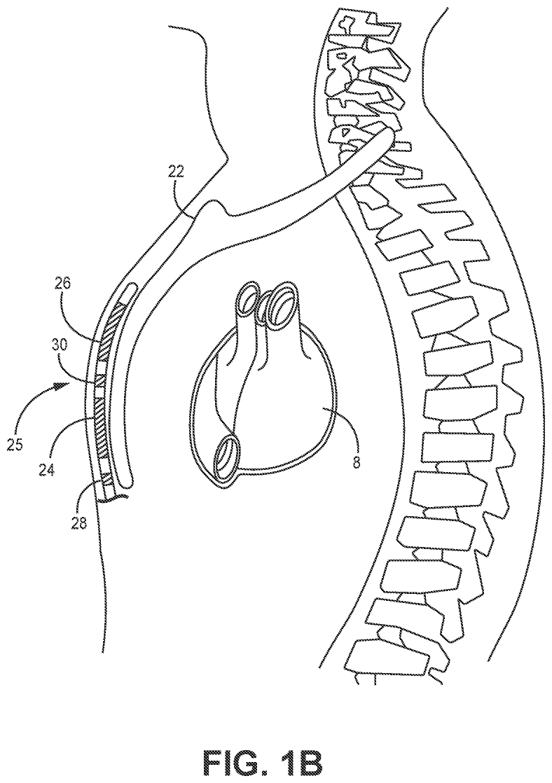

FIGS. 1A and 1B are conceptual diagrams of one example of an ICD system 10 that may be configured to sense cardiac electrical signals, detect arrhythmia and deliver electrical stimulation therapy according to the techniques disclosed herein. FIG. 1A is a front view of ICD system 10 implanted within patient 12. FIG. 1B is a side view of ICD system 10 implanted within patient 12. ICD system 10 includes an ICD 14 connected to an electrical stimulation and sensing lead 16, positioned in an extra-cardiovascular location in this example. FIGS. 1A and 1B are described in the context of an ICD system 10 capable of providing high voltage CV/DF shocks and/or cardiac pacing pulses in response to detecting a cardiac arrhythmia based on processing of sensed cardiac electrical signals. The techniques for detecting arrhythmia as disclosed herein may be implemented in a cardiac monitoring device that does not include cardiac pacing and/or CV/DF shock delivery capabilities in some examples. Furthermore, the techniques disclosed herein for sensing cardiac electrical signals and detecting arrhythmia may be implemented in a variety of medical devices including external or implantable cardiac monitors, pacemakers, and ICDs.

ICD 14 includes a housing 15 that forms a hermetic seal that protects internal components of ICD 14. The housing 15 of ICD 14 may be formed of a conductive material, such as titanium or titanium alloy. The housing 15 may function as an electrode (sometimes referred to as a “can” electrode). Housing 15 may be used as an active can electrode for use in delivering CV/DF shocks or other high voltage pulses delivered using a high voltage therapy circuit. In other examples, housing 15 may be available for use in delivering unipolar, relatively lower voltage cardiac pacing pulses and/or for sensing cardiac electrical signals in combination with electrodes carried by lead 16. In other instances, the housing 15 of ICD 14 may include a plurality of electrodes on an outer portion of the housing. The outer portion(s) of the housing 15 functioning as an electrode(s) may be coated with a material, such as titanium nitride, e.g., for reducing post-stimulation polarization artifact.

ICD 14 includes a connector assembly 17 (also referred to as a connector block or header) that includes electrical feedthroughs crossing housing 15 to provide electrical connections between conductors extending within the lead body 18 of lead 16 and electronic components included within the housing 15 of ICD 14. As will be described in further detail herein, housing 15 may house one or more processing circuits, memories, transceivers, cardiac electrical signal sensing circuitry, therapy delivery circuitry, power sources and other components for sensing cardiac electrical signals, detecting a heart rhythm, and controlling and delivering electrical stimulation pulses to treat an abnormal heart rhythm.

Elongated lead body 18 has a proximal end 27 that includes a lead connector (not shown) configured to be connected to ICD connector assembly 17 and a distal portion 25 that includes one or more electrodes. In the example illustrated in FIGS. 1A and 1B, the distal portion 25 of lead body 18 includes defibrillation electrodes 24 and 26 and pace/sense electrodes 28 and 30. In some cases, defibrillation electrodes 24 and 26 may together form a defibrillation electrode in that they may be configured to be activated concurrently. Alternatively, defibrillation electrodes 24 and 26 may form separate defibrillation electrodes in which case each of the electrodes 24 and 26 may be activated independently.

Electrodes 24 and 26 (and in some examples housing 15) are referred to herein as “defibrillation electrodes” because they can be utilized, individually or collectively, for delivering high voltage stimulation therapy (e.g., CV/DF shocks). Electrodes 24 and 26 may be elongated coil electrodes and generally have a relatively high surface area for delivering high voltage electrical stimulation pulses compared to pacing and sensing electrodes 28 and 30. However, electrodes 24 and 26 and housing 15 may also be utilized to provide pacing functionality, sensing functionality or both pacing and sensing functionality in addition to or instead of high voltage stimulation therapy. In this sense, the use of the term “defibrillation electrode” herein should not be considered as limiting the electrodes 24 and 26 for use in only high voltage CV/DF shock therapy applications. For example, either of electrodes 24 and 26 may be used as a sensing electrode in a sensing electrode vector for sensing cardiac electrical signals and determining a need for an electrical stimulation therapy.

Electrodes 28 and 30 are relatively smaller surface area electrodes which are available for use in sensing electrode vectors for sensing cardiac electrical signals and may be used for delivering relatively low voltage pacing pulses in some configurations. Electrodes 28 and 30 are referred to as pace/sense electrodes because they are generally configured for use in low voltage applications, e.g., used as either a cathode or anode for delivery of pacing pulses and/or sensing of cardiac electrical signals, as opposed to delivering high voltage CV/DF shocks. In some instances, electrodes 28 and 30 may provide only pacing functionality, only sensing functionality or both.

ICD 14 may obtain cardiac electrical signals corresponding to electrical activity of heart 8 via a combination of sensing electrode vectors that include combinations of electrodes 24, 26, 28 and/or 30. In some examples, housing 15 of ICD 14 is used in combination with one or more of electrodes 24, 26, 28 and/or 30 in at least one sensing electrode vector. Various sensing electrode vectors utilizing combinations of electrodes 24, 26, 28, and 30 and housing 15 are described below for sensing one or more cardiac electrical signals. Each cardiac electrical signal that is sensed by ICD 14 may be sensed using a different sensing electrode vector, which may be selected by sensing circuitry included in ICD 14. As described herein, in some examples the cardiac electrical signal(s) received via a selected sensing electrode vector may be used by ICD 14 for sensing cardiac event signals attendant to intrinsic depolarizations of the myocardium, e.g., R-waves attendant to ventricular depolarization and in some cases P-waves attendant to atrial depolarization. Sensed cardiac event signals may be used for determining the heart rate and determining a need for cardiac pacing, e.g., for treating bradycardia or asystole for preventing a long ventricular pause, or for determining a need for tachyarrhythmia therapies, e.g., anti-tachycardia pacing (ATP) or CV/DF shocks.

In the example illustrated in FIGS. 1A and 1B, electrode 28 is located proximal to defibrillation electrode 24, and electrode 30 is located between defibrillation electrodes 24 and 26. One, two or more pace/sense electrodes may be carried by lead body 18. For instance, a third pace/sense electrode may be located distal to defibrillation electrode 26 in some examples. Electrodes 28 and 30 are illustrated as ring electrodes; however, electrodes 28 and 30 may comprise any of a number of different types of electrodes, including ring electrodes, short coil electrodes, hemispherical electrodes, directional electrodes, segmented electrodes, or the like. Electrodes 28 and 30 may be positioned at other locations along lead body 18 and are not limited to the positions shown. In other examples, lead 16 may include fewer or more pace/sense electrodes and/or defibrillation electrodes than the example shown here.

In the example shown, lead 16 extends subcutaneously or submuscularly over the ribcage 32 medially from the connector assembly 27 of ICD 14 toward a center of the torso of patient 12, e.g., toward xiphoid process 20 of patient 12. At a location near xiphoid process 20, lead 16 bends or turns and extends superiorly, subcutaneously or submuscularly, over the ribcage and/or sternum, substantially parallel to sternum 22. Although illustrated in FIG. 1A as being offset laterally from and extending substantially parallel to sternum 22, the distal portion 25 of lead 16 may be implanted at other locations, such as over sternum 22, offset to the right or left of sternum 22, angled laterally from sternum 22 toward the left or the right, or the like. Alternatively, lead 16 may be placed along other subcutaneous or submuscular paths. The path of extra-cardiovascular lead 16 may depend on the location of ICD 14, the arrangement and position of electrodes carried by the lead body 18, and/or other factors. The techniques disclosed herein are not limited to a particular path of lead 16 or final locations of electrodes 24, 26, 28 and 30.

Electrical conductors (not illustrated) extend through one or more lumens of the elongated lead body 18 of lead 16 from the lead connector at the proximal lead end 27 to electrodes 24, 26, 28, and 30 located along the distal portion 25 of the lead body 18. The elongated electrical conductors contained within the lead body 18, which may be separate respective insulated conductors within the lead body 18, are each electrically coupled with respective defibrillation electrodes 24 and 26 and pace/sense electrodes 28 and 30. The respective conductors electrically couple the electrodes 24, 26, 28, and 30 to circuitry, such as a therapy delivery circuit and/or a sensing circuit, of ICD 14 via connections in the connector assembly 17, including associated electrical feedthroughs crossing housing 15. The electrical conductors transmit electrical stimulation pulses from a therapy delivery circuit within ICD 14 to one or more of defibrillation electrodes 24 and 26 and/or pace/sense electrodes 28 and 30 and transmit electrical signals produced by the patient's heart 8 from one or more of defibrillation electrodes 24 and 26 and/or pace/sense electrodes 28 and 30 to the sensing circuit within ICD 14.

The lead body 18 of lead 16 may be formed from a non-conductive material, including silicone, polyurethane, fluoropolymers, mixtures thereof, and/or other appropriate materials, and shaped to form one or more lumens within which the one or more conductors extend. Lead body 18 may be tubular or cylindrical in shape. In other examples, the distal portion 25 (or all of) the elongated lead body 18 may have a flat, ribbon or paddle shape. Lead body 18 may be formed having a preformed distal portion 25 that is generally straight, curving, bending, serpentine, undulating or zig-zagging.

In the example shown, lead body 18 includes a curving distal portion 25 having two “C” shaped curves, which together may resemble the Greek letter epsilon, “ε.” Defibrillation electrodes 24 and 26 are each carried by one of the two respective C-shaped portions of the lead body distal portion 25. The two C-shaped curves are seen to extend or curve in the same direction away from a central axis of lead body 18, along which pace/sense electrodes 28 and 30 are positioned. Pace/sense electrodes 28 and 30 may, in some instances, be approximately aligned with the central axis of the straight, proximal portion of lead body 18 such that mid-points of defibrillation electrodes 24 and 26 are laterally offset from pace/sense electrodes 28 and 30.

Other examples of extra-cardiovascular leads that may be implemented with the techniques described herein may include one or more defibrillation electrodes and one or more pacing and sensing electrodes carried by curving, serpentine, undulating or zig-zagging distal portion of the lead body 18. The techniques disclosed herein are not limited to any particular lead body design. In other examples, lead body 18 is a flexible elongated lead body without any pre-formed shape, bends or curves.

ICD 14 analyzes the cardiac electrical signal(s) received from one or more sensing electrode vectors to monitor for abnormal rhythms, such as asystole, bradycardia, ventricular tachycardia (VT) and/or ventricular fibrillation (VF). ICD 14 may analyze the heart rate and/or morphology of the cardiac electrical signals to monitor for a long pause (e.g., due to asystole or bradycardia) and ventricular tachyarrhythmia in accordance with techniques disclosed herein. ICD 14 generates and delivers electrical stimulation therapy in response to detecting a tachyarrhythmia, e.g., VT or VF (VT/VF), using a therapy delivery electrode vector which may be selected from any of the available electrodes 24, 26, 28 30 and/or housing 15. ICD 14 may deliver ATP in response to VT detection and in some cases may deliver ATP prior to a CV/DF shock or during high voltage capacitor charging in an attempt to avert the need for delivering a CV/DF shock. If ATP does not successfully terminate VT or when VF is detected, ICD 14 may deliver one or more CV/DF shocks via one or both of defibrillation electrodes 24 and 26 and/or housing 15.

In the absence of a sensed ventricular event signal, e.g., when a long pause in ventricular activity or asystole is detected, ICD 14 may generate and deliver a cardiac pacing pulse, such as a post-shock pacing pulse or bradycardia pacing pulse. The cardiac pacing pulses may be delivered using a pacing electrode vector that includes one or more of the electrodes 24, 26, 28, and 30 and the housing 15 of ICD 14.

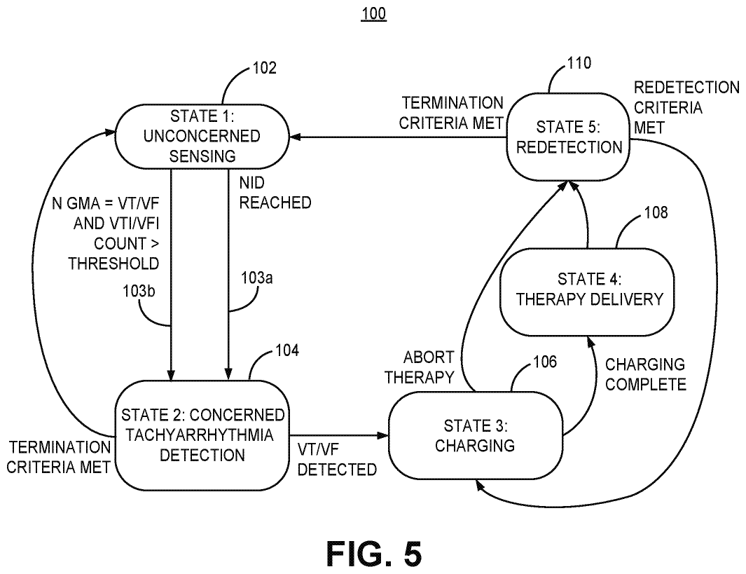

As described below, at least one sensing electrode vector may be selected for sensing a cardiac signal segment over a predetermined time interval to classify the cardiac signal segment as asystole, non-ventricular tachyarrhythmia or ventricular tachyarrhythmia based on a morphology analysis. The morphology analysis of a cardiac signal segment may be triggered when a long pause in ventricular activity is suspected based on a first analysis of sensed ventricular event signals according to a bradycardia sensing method. Additionally or alternatively, the morphology analysis of a cardiac signal segment may be triggered when undersensing of tachyarrhythmia (e.g., undersensing of fibrillation waves or low amplitude R-waves) is suspected based on a second analysis of sensed ventricular event signals according to a tachyarrhythmia sensing method. In still other examples, the morphology analysis may be triggered when ICD 14 transitions from one tachyarrhythmia operating state to another tachyarrhythmia operating state, e.g., as described below in conjunction with FIG. 5.

When the morphology analysis is triggered, the cardiac signal segment may be classified as a ventricular tachyarrhythmia segment (also referred to herein as a “VT/VF segment”), which may cause ICD 14 to increase its sensitivity for sensing ventricular event signals and inhibit ventricular pacing. If the cardiac signal segment is not classified as a VT/VF segment, e.g., a non-VT/VF segment or an asystole segment, this classification may be used for enabling ventricular pacing delivery. In some examples, ICD 14 is configured to detect VT/VF when a rejection rule is not met and a required number of VT/VF intervals have been detected based on sensed ventricular event signals. A rejection rule may include criteria applied to cardiac electrical signals sensed by ICD 14 for withholding a VT/VF detection when oversensing of P-waves, T-waves or non-cardiac noise is likely to be occurring and/or when an SVT is likely to be present, any of which may cause a false VT/VF detection. When a cardiac signal segment is classified as VT/VF, a rejection rule may be ignored or disregarded so that VT/VF detection is not withheld, even when other criteria applied for assessing the rejection rule are met. When a cardiac signal segment is not classified as VT/VF (e.g., classified as non-VT/VF or asystole) based on the morphology analysis, IMD 14 may withhold detection of VT/VF if a rejection rule is satisfied even when a required number of VT/VF intervals for detecting VT/VF have been counted.

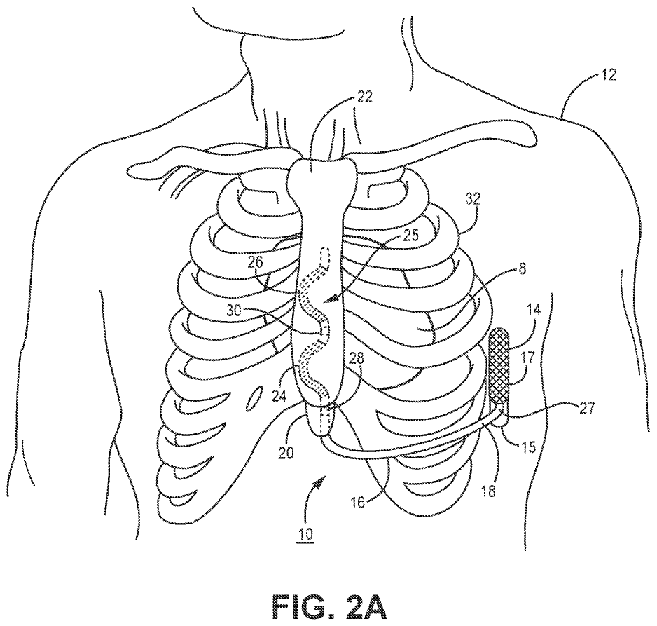

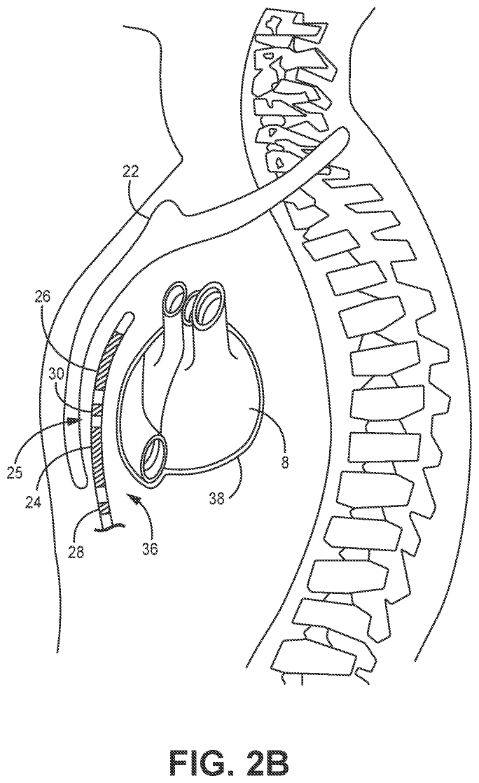

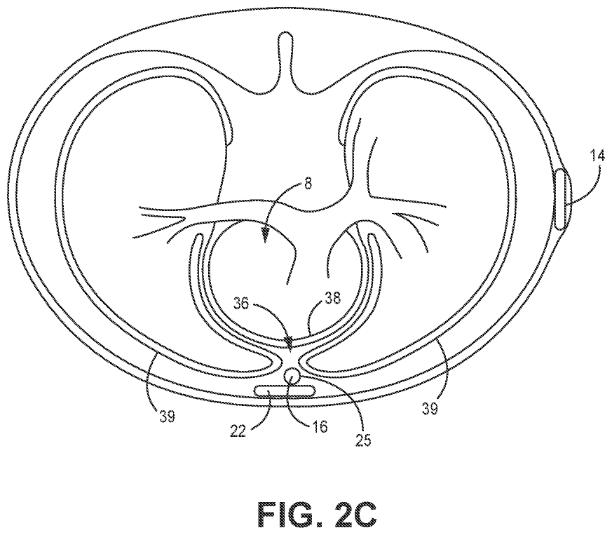

ICD 14 is shown implanted subcutaneously on the left side of patient 12 along the ribcage 32. ICD 14 may, in some instances, be implanted between the left posterior axillary line and the left anterior axillary line of patient 12. ICD 14 may, however, be implanted at other subcutaneous or submuscular locations in patient 12. For example, ICD 14 may be implanted in a subcutaneous pocket in the pectoral region. In this case, lead 16 may extend subcutaneously or submuscularly from ICD 14 toward the manubrium of sternum 22 and bend or turn and extend inferiorly from the manubrium to the desired location subcutaneously or submuscularly. In yet another example, ICD 14 may be placed abdominally. Lead 16 may be implanted in other extra-cardiovascular locations as well. For instance, as described with respect to FIGS. 2A-2C, the distal portion 25 of lead 16 may be implanted underneath the sternum/ribcage in the substernal space. FIGS. 1A and 1B are illustrative in nature and should not be considered limiting in the practice of the techniques disclosed herein.

A medical device operating according to techniques disclosed herein may be coupled to a transvenous or non-transvenous lead in various examples for carrying electrodes for sensing cardiac electrical signals and delivering electrical stimulation therapy. For example, the medical device, such as ICD 14, may be coupled to an extra-cardiovascular lead as illustrated in the accompanying drawings, referring to a lead that positions electrodes outside the blood vessels, heart, and pericardium surrounding the heart of a patient. Implantable electrodes carried by extra-cardiovascular leads may be positioned extra-thoracically (outside the ribcage and sternum), subcutaneously or submuscularly, or intra-thoracically (beneath the ribcage or sternum, sometimes referred to as a sub-sternal position) and may not necessarily be in intimate contact with myocardial tissue. An extra-cardiovascular lead may also be referred to as a “non-transvenous” lead.

In other examples, the medical device may be coupled to a transvenous lead that positions electrodes within a blood vessel, which may remain outside the heart in an “extra-cardiac” location or be advanced to position electrodes within a heart chamber. For instance, a transvenous medical lead may be advanced along a venous pathway to position electrodes in an extra-cardiac location within the internal thoracic vein (ITV), an intercostal vein, the superior epigastric vein, or the azygos, hemiazygos, or accessory hemiazygos veins, as examples. In still other examples, a transvenous lead may be advanced to position electrodes within the heart, e.g., within an atrial and/or ventricular heart chambers.

An external device 40 is shown in telemetric communication with ICD 14 by a wireless communication link 42 in FIG. 1A. External device 40 may include a processor 52, memory 53, display 54, user interface 56 and telemetry unit 58. Processor 52 controls external device operations and processes data and signals received from ICD 14. Display unit 54, which may include a graphical user interface, displays data and other information to a user for reviewing ICD operation and programmed parameters as well as cardiac electrical signals retrieved from ICD 14.

User interface 56 may include a mouse, touch screen, keypad or the like to enable a user to interact with external device 40 to initiate a telemetry session with ICD 14 for retrieving data from and/or transmitting data to ICD 14, including programmable parameters for controlling cardiac event signal sensing, arrhythmia detection and therapy delivery. Telemetry unit 58 includes a transceiver and antenna configured for bidirectional communication with a telemetry circuit included in ICD 14 and is configured to operate in conjunction with processor 52 for sending and receiving data relating to ICD functions via communication link 42.

Communication link 42 may be established between ICD 14 and external device 40 using a radio frequency (RF) link such as BLUETOOTH®, Wi-Fi, or Medical Implant Communication Service (MICS) or other RF or communication frequency bandwidth or communication protocols. Data stored or acquired by ICD 14, including physiological signals or associated data derived therefrom, results of device diagnostics, battery status, and histories of detected rhythm episodes and delivered therapies, etc., may be retrieved from ICD 14 by external device 40 following an interrogation command.

External device 40 may be embodied as a programmer used in a hospital, clinic or physician's office to retrieve data from ICD 14 and to program operating parameters and algorithms in ICD 14 for controlling ICD functions. External device 40 may alternatively be embodied as a home monitor or handheld device. External device 40 may be used to program cardiac signal sensing parameters, cardiac rhythm detection parameters and therapy control parameters used by ICD 14. At least some control parameters used in sensing cardiac event signals and detecting arrhythmias according to the techniques disclosed herein as well as therapy delivery may be programmed into ICD 14 using external device 40 in some examples.

FIGS. 2A-2C are conceptual diagrams of patient 12 implanted with extra-cardiovascular ICD system 10 in a different implant configuration than the arrangement shown in FIGS. 1A-1B. FIG. 2A is a front view of patient 12 implanted with ICD system 10. FIG. 2B is a side view of patient 12 implanted with ICD system 10. FIG. 2C is a transverse view of patient 12 implanted with ICD system 10. In this arrangement, extra-cardiovascular lead 16 of system 10 is implanted at least partially underneath sternum 22 of patient 12. Lead 16 extends subcutaneously or submuscularly from ICD 14 toward xiphoid process 20 and at a location near xiphoid process 20 bends or turns and extends superiorly within anterior mediastinum 36 (see FIG. 2C) in a substernal position.

Anterior mediastinum 36 may be viewed as being bounded laterally by pleurae 39, posteriorly by pericardium 38, and anteriorly by sternum 22 (see FIG. 2C). The distal portion 25 of lead 16 may extend along the posterior side of sternum 22 substantially within the loose connective tissue and/or substernal musculature of anterior mediastinum 36. A lead implanted such that the distal portion 25 is substantially within anterior mediastinum 36, may be referred to as a “substernal lead.”

In the example illustrated in FIGS. 2A-2C, lead 16 is located substantially centered under sternum 22. In other instances, however, lead 16 may be implanted such that it is offset laterally from the center of sternum 22. In some instances, lead 16 may extend laterally such that distal portion 25 of lead 16 is underneath/below the ribcage 32 in addition to or instead of sternum 22. In other examples, the distal portion 25 of lead 16 may be implanted in other extra-cardiac, intra-thoracic locations, including in the pleural cavity or around the perimeter of and adjacent to the pericardium 38 of heart 8.

In the various example implant locations of lead 16 and electrodes 24, 26, 28 and 30 shown and described herein, cardiac signals sensed by ICD 14 may have a relatively low and/or variable signal strength, e.g., caused by postural changes, respiration or other body movement, and/or may be contaminated by skeletal muscle myopotentials and/or environmental EMI. Undersensing of R-waves or fibrillation waves may result in an undetected tachyarrhythmia when ATP or CV/DF therapy may be needed. Oversensing of P-waves, T-waves, skeletal muscle myopotentials or other noise may lead to a false tachyarrhythmia detection resulting in unnecessary ATP or CV/DF shock delivery. In other instances, oversensing of cardiac signals (e.g., falsely sensing P-waves or T-waves as being R-waves) or non-cardiac noise (skeletal muscle myopotentials, EMI or other electrical noise) may result in withholding of pacing pulses when cardiac pacing is needed to prevent a long ventricular pause or asystole. Undersensing of R-waves or fibrillation waves may cause unneeded ventricular pacing pulse delivery that could confound VT/VF detection. Techniques disclosed herein provide improvements in sensing ventricular event signals (e.g., R-waves) and detecting arrhythmias by an implantable medical device. Improvements in sensing and detecting arrhythmias with high sensitivity and specificity can improve the performance of the implantable medical device in delivering appropriate electrical stimulation therapy for successfully treating the detected arrhythmia.

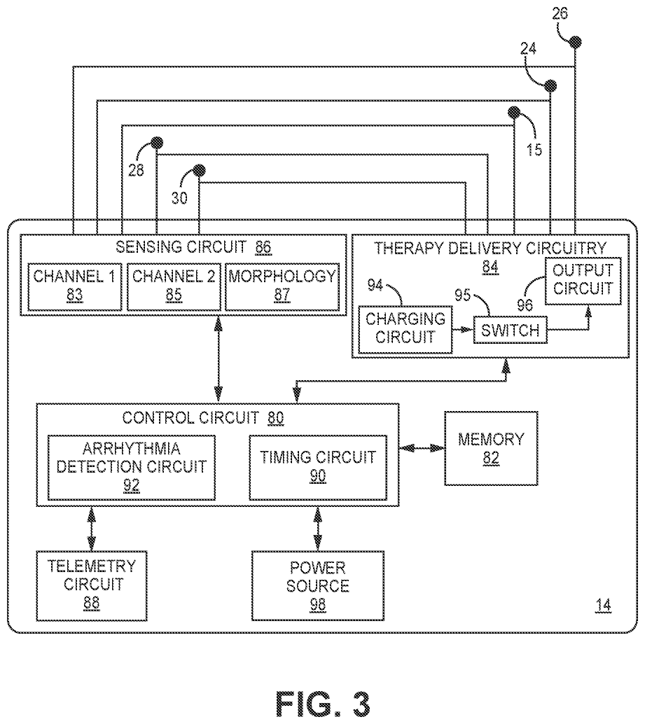

FIG. 3 is a conceptual diagram of ICD 14 according to one example. The electronic circuitry enclosed within housing 15 (shown schematically as an electrode in FIG. 3) may include software, firmware and/or hardware that cooperatively monitor cardiac electrical signals, determine when an electrical stimulation therapy is necessary, and deliver therapy as needed according to programmed therapy delivery algorithms and control parameters. ICD 14 may be coupled to a lead, such as lead 16 carrying electrodes 24, 26, 28, and 30, for delivering electrical stimulation pulses to the patient's heart and for sensing cardiac electrical signals.

ICD 14 includes a control circuit 80, memory 82, therapy delivery circuit 84, cardiac electrical signal sensing circuit 86, and telemetry circuit 88. A power source 98 provides power to the circuitry of ICD 14, including each of the components 80, 82, 84, 86, and 88 as needed. Power source 98 may include one or more energy storage devices, such as one or more rechargeable or non-rechargeable batteries. The connections between power source 98 and each of the other components 80, 82, 84, 86 and 88 are to be understood from the general block diagram of FIG. 3 but are not shown for the sake of clarity. For example, power source 98 may be coupled to one or more charging circuits included in therapy delivery circuit 84 for charging holding capacitors included in therapy delivery circuit 84 that are discharged at appropriate times under the control of control circuit 80 for producing electrical pulses according to a therapy protocol. Power source 98 is also coupled to components of cardiac electrical signal sensing circuit 86, such as sense amplifiers, analog-to-digital converters, switching circuitry, etc. as needed.

The circuits shown in FIG. 3 represent functionality included in ICD 14 and may include any discrete and/or integrated electronic circuit components that implement analog and/or digital circuits capable of producing the functions attributed to ICD 14 herein. Functionality associated with one or more circuits may be performed by separate hardware, firmware and/or software components, or integrated within common hardware, firmware and/or software components. For example, cardiac electrical signal sensing and analysis for detecting arrhythmia may be performed cooperatively by sensing circuit 86 and control circuit 80 and may include operations implemented in a processor or other signal processing circuitry included in sensing circuit 86 and/or control circuit 80 executing instructions stored in memory 82 and control signals such as blanking and timing intervals and sensing threshold amplitude signals sent from control circuit 80 to sensing circuit 86.

Control circuit 80 may include hardware configured to perform subroutines of signal processing and analysis techniques disclosed herein to reduce the processing burden associated with firmware and/or software execution of processing routines. For example hardware subroutines (HSRs) may be implemented in control circuit 80 to perform specific processing functions such as dedicated math operations, which may include any of sum, absolute value, difference, extrema, histogram counts, signal filtering (e.g., biquad filter, difference filter or other filters), etc. These HSRs could be called by control circuit firmware when processing and analyzing a cardiac signal for detecting arrhythmia, which may include a low pass filter, difference filter, gradient filter or other signal processing. HSRs may be called when control circuit 80 is determining various morphology parameters from a cardiac signal for detecting arrhythmia as described herein, which may include any of a mean period, spectral width, low slope content, signal pulse amplitudes, signal pulse intervals, etc. These HSRs can unload the processing burden associated with firmware and/or software processing to reduce current drain of power source 98 and thereby extend the useful life of ICD 14.

The various circuits of ICD 14 may include an application specific integrated circuit (ASIC), an electronic circuit, a processor (shared, dedicated, or group) and memory that execute one or more software or firmware programs, a combinational logic circuit, state machine, HSR, or other suitable components or combinations of components that provide the described functionality. The particular form of software, hardware and/or firmware employed to implement the functionality disclosed herein will be determined primarily by the particular system architecture employed in the ICD and by the particular sensing, detection and therapy delivery methodologies employed by the ICD. Providing software, hardware, and/or firmware to accomplish the described functionality in the context of any modern medical device system, given the disclosure herein, is within the abilities of one of skill in the art.

Memory 82 may include any volatile, non-volatile, magnetic, or electrical non-transitory computer readable storage media, such as random access memory (RAM), read-only memory (ROM), non-volatile RAM (NVRAM), electrically-erasable programmable ROM (EEPROM), flash memory, or any other memory device. Furthermore, memory 82 may include non-transitory computer readable media storing instructions that, when executed by one or more processing circuits, cause control circuit 80 and/or other ICD components to perform various functions attributed to ICD 14 or those ICD components. The non-transitory computer-readable media storing the instructions may include any of the media listed above.

Control circuit 80 communicates, e.g., via a data bus, with therapy delivery circuit 84 and sensing circuit 86 for sensing cardiac electrical signals, detecting cardiac rhythms, and controlling delivery of cardiac electrical stimulation therapies in response to sensed cardiac signals. Therapy delivery circuit 84 and sensing circuit 86 may be electrically coupled to electrodes 24, 26, 28, 30 carried by lead 16 and/or the housing 15, which may function as a common or ground electrode or as an active can electrode for delivering CV/DF shock pulses or cardiac pacing pulses.

Cardiac electrical signal sensing circuit 86 (also referred to herein as “sensing circuit” 86) may be selectively coupled to electrodes 28, 30 and/or housing 15 in order to monitor electrical activity of the patient's heart. Sensing circuit 86 may additionally be selectively coupled to defibrillation electrodes 24 and/or 26 for use in a sensing electrode vector together or in combination with one or more of electrodes 28, 30 and/or housing 15. Sensing circuit 86 may be enabled to receive cardiac electrical signals from at least one sensing electrode vector selected from the available electrodes 24, 26, 28, 30, and housing 15 in some examples. At least two, three or more cardiac electrical signals from two, three or more different sensing electrode vectors may be received simultaneously by sensing circuit 86 in some examples. Sensing circuit 86 may monitor one or more cardiac electrical signals for sensing cardiac event signals, e.g., R-waves attendant to intrinsic ventricular myocardial depolarizations. In some examples, sensing circuit 86 may be configured to monitor two cardiac electrical signals simultaneously for sensing cardiac event signals. At least one cardiac electrical signal may be received by sensing circuit 86 and passed to control circuit 80 for processing and analysis for determining when morphology-based criteria for detecting an arrhythmia are met. As described below, a cardiac electrical signal received over a predetermined or specified time interval may be analyzed for classifying the signal segment as being VT/VF, non-VT/VF or asystole. In the example shown, sensing circuit 86 may include switching circuitry for selecting which of electrodes 24, 26, 28, 30, and housing 15 are coupled as a first sensing electrode vector to a first sensing channel 83 for receiving a first cardiac electrical signal, which electrodes are coupled as a second sensing electrode vector to a second sensing channel 85 of sensing circuit 86 for receiving a second cardiac electrical signal, and which electrodes are coupled as a third sensing electrode vector to a morphology signal channel 87 for receiving a third cardiac electrical signal.

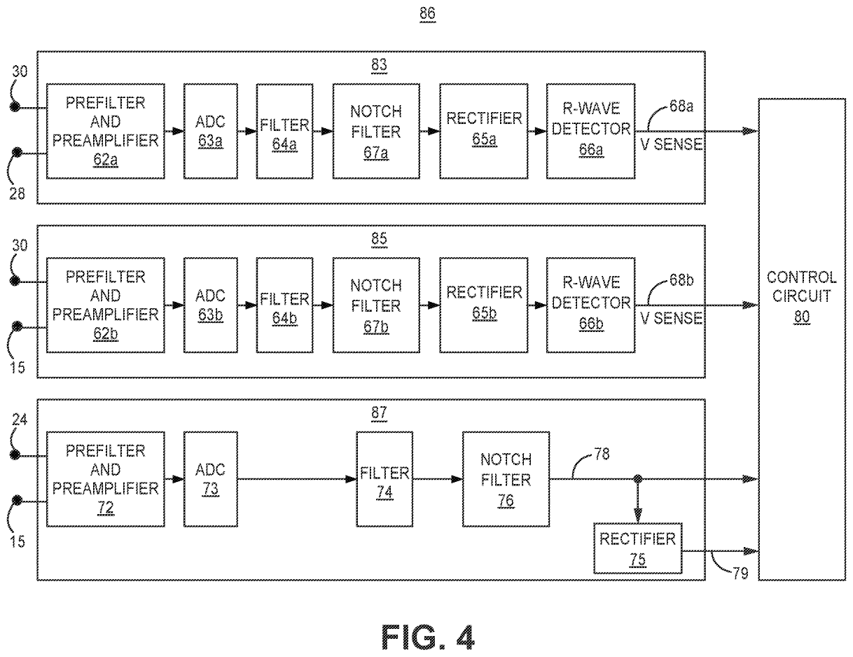

Each sensing channel 83 and 85, when both are included, may be configured to amplify, filter and digitize the cardiac electrical signal received from selected electrodes coupled to the respective sensing channel to improve the signal quality for sensing cardiac event signals, such as R-waves. The cardiac event detection circuitry within sensing circuit 86 may include one or more sense amplifiers, filters, rectifiers, threshold detectors, comparators, analog-to-digital converters (ADCs), timers or other analog and/or digital components as described further in conjunction with FIG. 4. A cardiac event sensing threshold may be automatically adjusted by each sensing channel 83 and 85 under the control of control circuit 80, based on sensing threshold control parameters, such as various timing intervals and sensing threshold amplitude values that may be determined by control circuit 80, stored in memory 82, and/or controlled by hardware, firmware and/or software of control circuit 80 and/or sensing circuit 86.

First sensing channel 83 and second sensing channel 85 may each control a cardiac event sensing threshold, e.g., an R-wave sensing threshold, that is applied to the incoming cardiac electrical signal for sensing cardiac event signals, e.g., R-waves. Upon sensing a cardiac event signal based on a sensing threshold crossing, first sensing channel 83 may produce a sensed event signal that is passed to control circuit 80. For example, upon detecting an R-wave sensing threshold crossing by the cardiac electrical signal received via a first sensing electrode vector, the first sensing channel 83 may generate a ventricular sensed event (Vsense) signal that is passed to control circuit 80. Similarly, upon detecting an R-wave sensing threshold crossing by a second cardiac electrical signal received by second sensing channel 85, the second sensing channel 85 may generate a Vsense signal that is passed to control circuit 80. The first and second sensing channels 83 and 85 may be configured to automatically adjust the R-wave sensing threshold used by each channel separately. The Vsense signals and relative timing from each other may be used by control circuit 80 for determining sensed event intervals for use in detecting VT/VF and/or controlling pacing pulse delivery.

Vsense signals received from sensing circuit 86 by control circuit 80 can be used by control circuit 80 for determining sensed event intervals, which are referred to herein as RR intervals (RRIs). An RRI is the time interval between two Vsense signals received by control circuit 80 from the same sensing channel 83 or 85, which may also be referred to as an “in-channel” sensed event interval. Control circuit 80 may include a timing circuit 90 for determining RRIs between consecutive Vsense signals received from a given sensing channel 83 or 85. Based on RRIs, control circuit 80 may detect VT/VF in some examples. In some instances, when a Vsense signal is received following a delivered pacing pulse, the RRI is determined from the pacing pulse to the Vsense signal. As such, RRIs may include time intervals between consecutive Vsense signals and intervals between a delivered pacing pulse and a Vsense signal.

Illustrative techniques disclosed herein are described in conjunction with sensing circuit 86 configured to receive two different cardiac electrical signals by the two cardiac event sensing channels 83 and 85 for sensing R-waves from the two cardiac electrical signals and for receiving a third cardiac electrical signal by morphology signal channel 87 for passing a digitized electrocardiogram (ECG) signal to control circuit 80 for morphology analysis. The three cardiac electrical signals sensed by sensing circuit 86 may be received using three different sensing electrode vectors selected from the available electrodes 24, 26, 28 and 30 and housing 15. In other examples, two cardiac electrical signals may be received by sensing circuit 86 from two different sensing electrode vectors, with one signal passed to the first sensing channel 83 and the other signal passed to the second sensing channel 85. Either or both of the two signals may be passed to control circuit 80 as a multi-bit digital ECG signal used by control circuit 80 for morphology analysis for analysis of a predetermined time segment of the ECG signal for detecting asystole and tachyarrhythmia according to the techniques disclosed herein.

Timing circuit 90 may be configured to control various timers and/or counters used in setting various intervals and windows used in sensing ventricular event signals, determining time intervals between received Vsense signals, performing morphology analysis and controlling the timing of cardiac pacing pulses generated by therapy delivery circuit 84. Timing circuit 90 may start a timer in response to receiving Vsense signals from sensing channels 83 and 85 for timing the RRIs between consecutively received in-channel Vsense signals (and in some instances from a delivered pacing pulse to a Vsense signal). Control circuit 80 may pass the RRI to arrhythmia detection circuit 92 for determining and counting tachyarrhythmia intervals.

Control circuit 80 may include an arrhythmia detection circuit 92 configured to analyze RRIs received from timing circuit 90 and cardiac electrical signals received from morphology signal channel 87 for detecting arrhythmia. Arrhythmia detection circuit 92 may be configured to detect a long ventricular pause (or asystole) and ventricular tachyarrhythmia based on sensed cardiac electrical signals meeting respective long pause (or asystole) or tachyarrhythmia detection criteria. For example, when a threshold number of Vsense signals from one sensing channel 83 or 85 each occur at a sensed event interval (RRI) that is less than a tachyarrhythmia detection interval, control circuit 80 may detect VT/VF. An RRI that is less than the tachyarrhythmia detection interval can be referred to as a “tachyarrhythmia interval” or a “VT/VF interval.”

In some examples, a tachyarrhythmia detection based on the threshold number of tachyarrhythmia intervals being reached may be confirmed or rejected based on morphology analysis of a cardiac electrical signal. As further described herein, one or more rejection rules relating to the possible oversensing of cardiac or non-cardiac events and/or possible SVT may be applied to cardiac electrical signals sensed by sensing circuit 86 for rejecting a VT/VF detection based on the threshold number of VT/VF intervals being reached for detecting VT/VF. Arrhythmia detection circuit 92 may perform RRI analysis and cardiac electrical signal analysis to detect VT/VF or withhold a VT/VF detection based on a combination of VT/VF interval counts, morphology analysis of cardiac signal segments and one or more rejection rules as further described in conjunction with the accompanying flow charts and diagrams presented herein.

Arrhythmia detection circuit 92 may be implemented in control circuit 80 as hardware, software and/or firmware that processes and analyzes signals received from sensing circuit 86 for detecting arrhythmia, including a long pause in ventricular activity or asystole and VT/VF. Arrhythmia detection circuit 92 may identify signal pulses for determining amplitude and/or pulse interval metrics of a cardiac electrical signal segment for use in detecting arrhythmia as further described below. Arrhythmia detection circuit 92 may be configured to determine morphology metrics of cardiac signal segments that are correlated to the signal amplitude, stability, slope content, and/or frequency content of the cardiac signal segment(s) in some examples. The morphology metrics may be compared to criteria for detecting a long pause or asystole or for detecting VT/VF for enabling therapy delivery circuit 84 to appropriately deliver (or inhibit) cardiac pacing and/or CV/DF shock therapy in response to an arrhythmia detection.

In some examples, arrhythmia detection circuit 92 may include comparators and counters for counting RRIs determined by timing circuit 90 from Vsense signals received from sensing channel 83 and/or sensing channel 85 that fall into various rate detection zones for determining a ventricular rate or performing other rate-or interval-based assessment of Vsense signals for detecting and discriminating VT and VF. For example, arrhythmia detection circuit 92 may compare the RRIs determined by timing circuit 90 to one or more tachyarrhythmia detection interval zones, such as a tachycardia detection interval zone and a fibrillation detection interval zone. RRIs falling into a detection interval zone can be counted by a respective VT interval (VTI) counter or VF interval (VFI) counter and in some cases in a combined VT/VF interval counter. The VF detection interval threshold may be set to 300 to 350 milliseconds (ms), as an example. For instance, if the VF detection interval is set to 320 ms, RRIs that are less than 320 ms are counted by the VFI counter. When VT detection is enabled, the VT detection interval may be programmed to be in the range of 350 to 420 ms, or 400 ms as an example. RRIs that are less than the VT detection interval but greater than or equal to the VF detection interval may be counted by a VTI counter. VT or VF may be detected when the respective VT or VFI counter (or a combined VT/VF interval counter) reaches a threshold number of intervals to detect (NID) and any other VT/VF detection criteria are met as described herein.

As an example, the NID to detect VT may require that the VTI counter reaches 18 VTIs, 24 VTIs, 32 VTIs or other selected NID. In some examples, the VTIs may be required to be consecutive intervals, e.g., 18 out of 18, 24 out of 24, or 32 out of 32 or 100 out of the most recent 100 consecutive RRIs. The NID required to detect VF may be programmed to a threshold number of X VFIs out of Y consecutive RRIs. For instance, the NID required to detect VF may be 18 VFIs out of the most recent 24 consecutive RRIs, 30 VFIs out 40 consecutive RRIs, or as high as 120 VFIs out of 160 consecutive RRIs as examples (or other percentage of a specified number of RRIs). When a VTI or VFI counter reaches a respective NID, a ventricular tachyarrhythmia may be detected by arrhythmia detection circuit 92. The NID may be programmable and range from as low as 12 to as high as 120, with no limitation intended. A VTI counter or VFI counter may reach a respective NID when VTIs or VFIs are detected consecutively or non-consecutively out of a specified number of most recent RRIs. In some cases, a combined VT/VF interval counter may count both VTIs and VFIs and detect a tachyarrhythmia episode based on the fastest intervals detected when a specified NID is reached.

Arrhythmia detection circuit 92 may be configured to perform other signal analysis for determining if other detection criteria are satisfied before detecting VT or VF based on an NID being reached, such as R-wave morphology criteria, onset criteria, stability criteria and noise and oversensing rejection criteria. As described below, arrhythmia detection circuit 92 (or generally control circuit 80) may perform morphology analysis of a cardiac signal segment for classifying the segment as being VT/VF, non-VT/VF or asystole in some examples. To support these additional analyses, sensing circuit 86 may pass a digitized ECG signal to control circuit 80, e.g., from morphology signal channel 87, for morphology analysis performed by arrhythmia detection circuit 92 for detecting and discriminating heart rhythms. The cardiac electrical signal received by control circuit 80 from morphology signal channel 87 is referred to herein a the “morphology signal.” A cardiac electrical signal received by the morphology signal channel 87 (and/or sensing channel 83 and/or sensing channel 85) may be passed through a filter and amplifier, provided to a multiplexer and thereafter converted to a multi-bit digital signal by an analog-to-digital converter, which may all be included in sensing circuit 86, for storage in memory 82. Memory 82 may include one or more circulating buffers to temporarily store digital cardiac signal segments for analysis performed by control circuit 80. Control circuit 80 may be a microprocessor-based controller, which may include HSRs, that employs digital signal analysis techniques to characterize the digitized signals stored in memory 82 to recognize and classify the patient's heart rhythm employing any of numerous signal processing methodologies for analyzing cardiac signals and cardiac event waveforms, e.g., R-waves.

Therapy delivery circuit 84 includes at least one charging circuit 94, including one or more charge storage devices such as one or more high voltage capacitors for generating high voltage shock pulses for treating VT/VF. Charging circuit 94 may include one or more low voltage capacitors for generating relatively lower voltage pulses, e.g., for cardiac pacing therapies. Therapy delivery circuit 84 may include switching circuitry 95 that controls when the charge storage device(s) are discharged through an output circuit 96 across a selected pacing electrode vector or CV/DF shock vector.

In response to detecting VT/VF, control circuit 80 may schedule a therapy and control therapy delivery circuit 84 to generate and deliver the therapy, such as ATP and/or CV/DF shock(s). Therapy can be generated by initiating charging of high voltage capacitors of charging circuit 94. Charging is controlled by control circuit 80 which monitors the voltage on the high voltage capacitors, which is passed to control circuit 80 via a charging control line. When the voltage reaches a predetermined value set by control circuit 80, a logic signal is generated on a capacitor full line and passed to therapy delivery circuit 84, terminating charging. A CV/DF shock pulse is delivered to the heart under the control of the timing circuit 90 by an output circuit 96 of therapy delivery circuit 84 via a control bus. The output circuit 96 may include an output capacitor or other output circuitry through which the charged high voltage capacitor is discharged via switching circuitry, e.g., an H-bridge, which determines the electrodes used for delivering the cardioversion or defibrillation pulse and the pulse wave shape. Therapy delivery circuit 84 may be configured to deliver electrical stimulation pulses for inducing tachyarrhythmia, e.g., T-wave shocks or trains of induction pulses, upon receiving a programming command from external device 40 (FIG. 1A) during ICD implant or follow-up testing procedures.

In some examples, the high voltage therapy circuit configured to deliver CV/DF shock pulses can be controlled by control circuit 80 to deliver pacing pulses, e.g., for delivering ATP, post shock pacing pulses, bradycardia pacing pulses or asystole pacing pulses. Therapy delivery circuit 84 may be configured to generate and deliver cardiac pacing pulses using the high voltage capacitor(s) that are chargeable to a shock voltage amplitude by charging the high voltage capacitor(s) to a relatively lower voltage corresponding to a cardiac pacing pulse amplitude for capturing and pacing the ventricular myocardium. Therapy delivery circuit 84 may include a low voltage therapy circuit including one or more separate or shared charging circuits, switch circuits and output circuits for generating and delivering relatively lower voltage pacing pulses for a variety of pacing needs. Charging of capacitors to a programmed pulse amplitude and discharging of the capacitors for a programmed pulse width may be performed by therapy delivery circuit 84 according to control signals received from control circuit 80 for delivering cardiac pacing pulses. As described above, timing circuit 90 may include various timers or counters that control when cardiac pacing pulses are delivered. The microprocessor of control circuit 80 may set the amplitude, pulse width, polarity or other characteristics of cardiac pacing pulses, which may be based on programmed values stored in memory 82.

Control parameters utilized by control circuit 80 for sensing cardiac event signals, detecting arrhythmias, and controlling therapy delivery may be programmed into memory 82 via telemetry circuit 88. Telemetry circuit 88 includes a transceiver and antenna for communicating with external device 40 (shown in FIG. 1A) using RF communication or other communication protocols as described above. Under the control of control circuit 80, telemetry circuit 88 may receive downlink telemetry from and send uplink telemetry to external device 40.

FIG. 4 is a conceptual diagram of circuitry that may be included in sensing circuit 86 shown in FIG. 3 according to some examples. Sensing circuit 86 may include a first sensing channel 83, second sensing channel 85 and morphology signal channel 87. First sensing channel 83 and second sensing channel 85 may each be selectively coupled via switching circuitry included in sensing circuit 86 to a respective sensing electrode vector including at least one electrode carried by extra-cardiovascular lead 16. First sensing channel 83 may be coupled to a first sensing electrode vector for receiving a first cardiac electrical signal, and second sensing channel 85 may be coupled to a second sensing electrode vector, different than the first sensing electrode vector for receiving a second cardiac electrical signal, different than the first cardiac electrical signal. In some examples, first sensing channel 83 may be coupled to a sensing electrode vector that is a short bipole, having a relatively shorter inter-electrode distance than the sensing electrode vector coupled to the second sensing channel 85 or to morphology signal channel 87. In the example shown, the first sensing channel 83 is coupled to pace/sense electrodes 28 and 30 carried by lead 16. In some examples, first sensing channel 83 may be coupled to a sensing electrode vector that is approximately vertical (when the patient is in an upright position) or approximately aligned with the cardiac axis to increase the likelihood of a relatively high R-wave signal amplitude relative to the P-wave signal amplitude. A relatively short inter-electrode distance, e.g., between electrodes 28 and 30 carried by lead 16, may be relatively less likely to be contaminated by skeletal muscle myopotential noise, EMI or other non-cardiac noise compared to a relatively longer inter-electrode distance but may have greater variability in R-wave signal strength compared to a relatively longer inter-electrode distance.

The second sensing channel 85 may be coupled to a second sensing electrode vector that is a short bipole or a relatively longer bipole compared to the first sensing electrode vector. The second sensing electrode vector may also be generally vertical or aligned with the cardiac axis. However, the second sensing electrode vector may be orthogonal or transverse relative to the first sensing electrode vector in other examples. In the example shown, the second sensing channel 85 is coupled to pace/sense electrode 30 and housing 15 such that it is a relatively longer bipole that is substantially transverse to the sensing electrode vector coupled to the first sensing channel 83. In other examples, the first or second sensing channels may be coupled to either of pace/sense electrodes 28 or 30 paired with housing 15, either of pace/sense electrodes 28 or 30 paired with coil electrode 24, or either of pace sense electrodes 28 or 30 paired with coil electrode 26, as long as at least one electrode is different between the two sensing electrode vectors. In further examples, either or both of first or second sensing channels 83 or 85 may be coupled to a sensing electrode vector that does not necessarily include one of pace/sense electrodes 28 or 30. For example, a sensing electrode vector may be coupled to sensing channel 83 or sensing channel 85 that includes one or both of coil electrodes 24 or 26 and/or housing 15.

Sensing circuit 86 may include a morphology signal channel 87 for sensing a third cardiac electrical signal. For instance, morphology signal channel 87 may receive a raw cardiac electrical signal from a third sensing electrode vector, for example from a vector that includes one electrode 24, 26, 28 or 30 carried by lead 16 paired with housing 15. Morphology signal channel 87 may be selectively coupled to a relatively long bipole having an inter-electrode distance or spacing that is greater than the sensing electrode vector coupled to first sensing channel 83 and/or second sensing channel 85 in some examples. The third sensing electrode vector may be, but not necessarily, approximately orthogonal to at least one of the first channel sensing electrode vector or the second channel sensing electrode vector. In the example shown, coil electrode 24 and housing 15 may be coupled to morphology signal channel 87 to provide the third sensed cardiac electrical signal. The third cardiac electrical signal received by morphology signal channel 87 may be used by control circuit 80 for morphology analysis for a variety of sensing and arrhythmia detection purposes. For example, control circuit 80 may perform a morphology analysis of a signal sensed by morphology signal channel 87 to determine when morphology-based tachyarrhythmia classification of a cardiac electrical signal segment occurs for use in controlling cardiac pacing, the sensitivity of sensing circuitry for ventricular event signal sensing, detecting VT/VF, detecting termination of VT/VF and/or redetecting VT/VF. In some examples, control circuit 80 may perform waveform morphology matching criteria for validating a Vsense signal for inhibiting a bradycardia pacing pulse. Various morphology analyses that may be performed by control circuit 80 using a signal received from morphology signal channel 87 are described below in conjunction with the accompanying flow charts and diagrams presented herein.

For these purposes, in some examples, the sensing electrode vector coupled to morphology signal channel 87 may provide a relatively far-field or more global cardiac signal compared to a relatively shorter bipole that may be coupled to the first sensing channel 83 or the second sensing channel 85. In other examples, any vector selected from the available electrodes, e.g., electrodes 24, 26, 28, 30 and/or housing 15, may be included in a sensing electrode vector coupled to morphology signal channel 87. The sensing electrode vectors coupled to first sensing channel 83 and second sensing channel 85 and, at least in some examples, morphology signal channel 87 may be different sensing electrode vectors, which may have no common electrodes or only one common electrode but not both electrodes in common between the different sensing electrode vectors. In other examples, however, the sensing electrode vector coupled to one of the first sensing channel 83 or the second sensing channel 85 may be the same sensing electrode vector coupled to the morphology signal channel 87. In this case, a sensing channel 83 or 85 and the morphology signal channel 87 may be combined or include shared components such that a morphology signal and Vsense signals may be output to control circuit 80 from one sensing channel.

The first sensing channel 83 and the second sensing channel 85 may each receive a cardiac electrical signal for sensing ventricular event signals attendant to ventricular myocardial depolarizations in response to the cardiac electrical signal crossing an R-wave sensing threshold. The morphology signal channel 87 may receive a third cardiac electrical signal for passing a multi-bit digital ECG signal to control circuit 80 for morphology analysis. In the illustrative example shown in FIG. 4, the signals received by first sensing channel 83, second sensing channel 85 and morphology signal channel 87 are provided as differential input signals to a pre-filter and pre-amplifier 62a, 62b, and 72, respectively. Non-physiological high frequency and DC signals may be filtered by a low pass or bandpass filter included in each of pre-filter and pre-amplifiers 62a, 62b and 72, and high voltage signals may be removed by protection diodes included in pre-filter and pre-amplifiers 62a, 62b and 72. Pre-filter and pre-amplifiers 62a, 62b and 72 may amplify the pre-filtered signal by a gain of between 10 and 100, and in one example a gain of 17, though each channel may have a different gain and filter bandwidth. Pre-filter and pre-amplifiers 62a, 62b and 72 may convert the differential input signal to a single-ended output signal passed to an analog-to-digital converter (ADC) 63a, 63b, and 73, respectively. Pre-filter and pre-amplifiers 62a, 62b and 72 may provide anti-alias filtering and noise reduction prior to digitization.

ADC 63a, ADC 63b and ADC 73, respectively, convert the first cardiac electrical signal, second cardiac electrical signal and third cardiac electrical signal from an analog signal to a digital bit stream, which may be sampled at 128 or 256 Hz, as examples. ADC 63a, ADC 63b and ADC 73 may be sigma-delta converters (SDC), but other types of ADCs may be used. In some examples, the outputs of ADC 63a, ADC 63b and ADC 73 may be provided to decimators (not shown), which function as digital low-pass filters that increase the resolution and reduce the sampling rate of the respective cardiac electrical signals.

The digital outputs of ADC 63a, ADC 63b and ADC 73 are each passed to respective filters 64a, 64b and 74, which may be digital bandpass filters. The bandpass filters 64a, 64b and 74 may have the same or different bandpass frequencies. For example, filters 64a and 64b may have a bandpass of approximately 10 Hz to 50 Hz, or approximately 13 Hz to 39 Hz, for passing cardiac electrical signals such as R-waves typically occurring in this frequency range. Filter 74 of the morphology signal channel 87 may have a relatively wider bandpass of approximately 2.5 to 100 Hz. In some examples, each of sensing channel 83, sensing channel 85 and morphology signal channel 87 may further include a notch filter 67a, 67b, and 76, respectively, to filter 50 Hz and 60 Hz noise signals. Each notch filter 67a, 67b, and 76 may be individually turned on or off in some examples.

The narrow bandpass and notch-filtered signal (if notch filter is turned on) in first sensing channel 83 and second sensing channel 85 is passed from respective filter 64a or filter 64b (or 67a or 67b) to rectifier 65a or rectifier 65b to produce a filtered, rectified signal output to respective R-wave detectors 66a and 66b. First sensing channel 83 includes an R-wave detector 66a for sensing ventricular event signals in response to the first cardiac electrical signal crossing an R-wave sensing threshold. Second sensing channel 85 includes an R-wave detector 66b for sensing ventricular event signals in response to the second cardiac electrical signal crossing an R-wave sensing threshold, which may be controlled separately from the R-wave sensing threshold controlled by R-wave detector 66a, in some examples. R-wave detectors 66a and 66b may each include an auto-adjusting sense amplifier, comparator and/or other detection circuitry that compares the incoming filtered and rectified cardiac electrical signal to an R-wave sensing threshold and produces a Vsense signal 68a or 68b when the respective first or second cardiac electrical signal crosses the respective R-wave sensing threshold outside of a post-sense (or post-pace) blanking interval.

The R-wave sensing threshold may be a multi-level sensing threshold, e.g., as generally disclosed in U.S. Pat. No. 10,252,071 (Cao, et al.), incorporated herein by reference in its entirety. Briefly, the multi-level sensing threshold may have a starting sensing threshold value held for a first drop time interval, which may be equal to a tachycardia detection interval or an expected R-wave to T-wave interval, then drops to a second sensing threshold value held until a second drop time interval expires, which may be 0.6 to 2.5 seconds long in some examples, or 1 to 2.5 seconds long in other examples, and can be 2.15 seconds (from the Vsense signal) in one example. The R-wave sensing threshold may drop to the second sensing threshold in a single step decrement in some examples. After the second drop time interval, the sensing threshold drops to a minimum sensing threshold, which may be equal to a programmed sensitivity or an increased sensitivity based on morphology analysis of a cardiac signal segment. The increased sensitivity can be a sensitivity amplitude setting that is lower in amplitude (e.g., in millivolts) than the programmed sensitivity amplitude setting. The sensitivity is also referred to herein as the “sensing floor” because it represents the minimum amplitude of the cardiac electrical signal that may be sensed as a ventricular event signal, e.g., an R-wave or fibrillation wave. The R-wave sensing threshold may drop to the sensing floor e.g., to the programmed sensitivity or to an increased sensitivity (lower amplitude setting than the programmed sensitivity), in a single step decrement in some examples.

The R-wave sensing thresholds used by R-wave detector 66a and 66b may each be set to a starting value based on a maximum peak amplitude of the respective first or second cardiac electrical signal determined by the R-wave detector 66a or 66b during the most recent post-sense blanking interval. In some examples, an R-wave peak tracking period may be defined as a portion of the post-sense blanking period during which the maximum peak amplitude is determined. The starting R-wave sensing threshold of each sensing channel 83 and 85 may decrease over time according to one or more stepwise drops and/or linear or non-linear decay rates until reaching the minimum sensing threshold, e.g., equal to the sensitivity setting, or until an R-wave sensing threshold crossing by the cardiac electrical signal occurs. In some instances, the R-wave sensing threshold may be adjusted to the minimum sensing threshold (equal to the sensitivity setting) before the expiration of the first drop time interval or before the expiration of the second drop time interval depending on the maximum peak amplitude determined during the R-wave peak tracking period.

The techniques described herein are not limited to a specific behavior of the sensing threshold or specific R-wave sensing techniques. Instead, other decaying, step-wise adjusted or other automatically adjusted sensing thresholds may be utilized for sensing ventricular event signals from the respective first and second cardiac electrical signals. R-wave detector 66a or 66b may produce a Vsense signal 68a or 68b, respectively, in response to the respective first cardiac electrical signal or second cardiac electrical signal crossing the R-wave sensing threshold. The Vsense signal 68a or 68b is passed to control circuit 80.

The wideband-filtered, digital cardiac electrical signal 78 output from morphology signal channel 87 may be passed to control circuit 80 for performing morphology-based arrhythmia detection methods according to the techniques disclosed herein. In some examples, the digital cardiac electrical signal 78 is passed to rectifier 75 and a rectified wideband filtered signal 79 is passed to control circuit 80 for processing and analysis. In some cases, both the filtered, non-rectified signal 78 and the rectified signal 79 are passed to control circuit 80 from morphology signal channel 87 for use in determining morphology features of the ECG signal. As described below, an n-second ECG signal segment may be buffered in memory 82 by control circuit 80 for processing and analysis for classifying the segment, e.g., as one of asystole, VT/VF or non-VT/VF. The n-second ECG signal segment(s) analyzed by arrhythmia detection circuit 92 may undergo additional low pass, bandpass and/or high pass filtering and/or other signal processing prior to analysis for determining morphology features or other features of the ECG signal segment for arrhythmia detection.

The configuration of sensing channels 83 and 85 and morphology signal channel 87 as shown in FIG. 4 is illustrative in nature and should not be considered limiting of the techniques described herein. Sensing circuit 86 may include more or fewer components than illustrated and described in FIG. 4 and some components may be shared between sensing channels 83 and 85 and morphology signal channel 87. For example, a common cardiac electrical signal from a selected sensing electrode vector may be received by a prefilter and preamplifier circuit and ADC and subsequently be passed to a narrowband filter in one of sensing channels 83 or 85 and to a wideband filter in morphology signal channel 87. In other examples, sensing circuit 86 may include none, one or more than two sensing channels, each configured to produce a Vsense signal, and/or more than one morphology signal channel. In other examples, a wideband filtered morphology signal may be passed to control circuit 80 from one of sensing channels 83 or 85 for performing analysis of cardiac signal segments according to the techniques disclosed herein for use in detecting arrhythmia. Furthermore, the components for filtering, amplifying, digitizing, rectifying, etc. may be arranged in a different order or combination than shown in FIG. 4.

FIG. 5 is a conceptual diagram 100 of tachyarrhythmia operating states of ICD 14 relating to methods performed by ICD 14 for sensing and analyzing cardiac signals for detecting VT/VF and delivering therapy in response to a VT/VF detection according to some examples. At block 102, ICD 14 is operating in an unconcerned sensing state 1 (also referred to herein as “unconcerned state 1” or merely “state 1.” During the unconcerned sensing state 1, control circuit 80 receives Vsense signals from sensing circuit 86 and determines RRIs according to tachyarrhythmia sensing methods. In some examples, Vsense signals are received from each of sensing channels 83 and 85. Each sensing channel 83 and 85 may be initially sensing ventricular event signals according to a programmed sensitivity for each sensing channel, which may be a user programmed or default sensitivity setting and is referred to hereafter as the “programmed sensitivity.”

RRIs are determined by control circuit 80 between successively received, in-channel Vsense signals for each sensing channel 83 and 85. The RRIs are compared to a VT detection interval zone or threshold (when VT detection is enabled) and/or to a VF detection interval zone or threshold interval for identifying VTIs and/or VFIs. When an RRI is determined to be a VTI or VFI, a respective VTI counter or VFI counter (and/or combined VT/VF interval counter) designated for counting VTIs and VFIs identified for the respective sensing channel 83 or 85 is incremented. Thus each sensing channel 83 and 85 may be associated with a VTI counter, a VFI counter and/or a combined VT/VF interval counter. The VTI counter, VFI counter and a combined VT/VF interval counter, if used, are also referred to herein collectively as VTI/VFI counters. However, it is recognized that in some examples, VT detection may not be enabled in ICD 14 such that only VFI counters may be used for tracking VFIs for each sensing channel 83 and 85.

When a VTI or VFI counter for at least one sensing channel 83 or 85 reaches an NID required for detecting VT or VF, respectively, ICD 14 may transition (as indicated by arrow 103a) to the concerned tachyarrhythmia detection state 2 of block 104. ICD 14 may transition to the concerned tachyarrhythmia suspected state 2, also referred to herein as “concerned state 2” or simply “state 2,” at block 104 in response to the NID being reached when sensing circuit 86 is sensing ventricular event signals according to the programmed sensitivity setting for each sensing channel 83 and/or 85. As described below, in order to transition to the concerned tachyarrhythmia detection state 2, control circuit 80 may identify one sensing channel 83 or 85 as the reliable sensing channel for VT/VF detection when the NID is met for either sensing channel 83 or 85. The sensing channel 83 or 85 that is selected as the reliable sensing channel may be required to have reached an NID (by an associated VTI/VFI counter) in order to transition from state 1 of block 102 to state 2 of block 104. If the sensing channel 83 or 85 that is identified as the reliable sensing channel for detecting VT/VF is not the sensing channel associated with a VTI/VFI counter that has reached an NID, control circuit 80 may remain in the unconcerned sensing state 1 of block 102. Control circuit 80 may perform methods during the unconcerned sensing state 1 for sensing ventricular event signals using two sensing channels 83 and 85 and determining when criteria are met for transitioning to the concerned state 2 of block 104 as generally disclosed in U.S. patent application Ser. No. 17/823,055, filed Aug. 28, 2022 (Liu, et al.).

As further described below, during the unconcerned state 1, control circuit 80 may enable a gross morphology analysis (GMA) of a cardiac signal segment. “Gross morphology analysis,” or “GMA,” as used herein refers to an analysis performed by control circuit 80 of a cardiac signal segment that can begin and end independent of a timed relation to a Vsense signal received from sensing circuit 86. The cardiac signal segment undergoing GMA may have a total time duration that is greater than multiple VT detection intervals or multiple VF detection intervals. As such, the classification of the cardiac signal segment based on the GMA is not dependent on the timing, rate or intervals between Vsense signals. The classification of a cardiac signal segment based on the GMA can, therefore, be relatively immune to or independent of confounding factors of oversensing of ventricular event signals or the presence of SVT compared to rate or interval based VT/VF detection alone. By performing the GMA, VT/VF detection and discrimination of true VT/VF episodes from episodes of oversensing and SVT can be improved.

In performing the GMA, control circuit 80 determines at least one characteristic of the sample points of the cardiac signal segment without using or relying on the timing of any R-wave(s), fibrillation wave(s) or sensed event signals within the cardiac signal segment. The analysis may use sample points spanning the entire cardiac signal segment at a sampling interval for determining a morphology metric, for example. The analysis may determine at least one metric relating to the frequency content of the cardiac signal segment. The analysis may include determining a metric relating to a slope content of the cardiac signal segment. In some examples, the analysis may include determining a metric relating to the amplitude and/or noise content of the cardiac signal segment.

In some examples, when GMA is enabled during the unconcerned sensing state 1 and the first cardiac signal segment analyzed is classified as being VT/VF, control circuit 80 may continue the GMA of one or more subsequent cardiac signal segments. When a threshold number of cardiac signal segments are classified as being VT/VF based on the GMA and a VTI and/or VFI count is trending up toward the NID but has not yet reached the NID, control circuit 80 may transition from state 1 to state 2 (as indicated by arrow 103b). For example, to cause a transition to state 2 of block 104 as indicated by transition arrow 103b, the VTI/VFI count may be required to be greater than a threshold value that is 50%, 60%, 70%, 80% or 90% of the NID, but can be less than the NID, when at least 2, 3 or other specified number of cardiac signal segments are classified as VT/VF based on the GMA.

In other instances, control circuit 80 may increase the sensitivity of sensing channels 83 and 85 for sensing ventricular event signals during the unconcerned sensing state 1 of block 102 when at least one cardiac signal segment is classified as VT/VF based on the GMA of the cardiac signal segment. The NID may be reached after increasing the sensitivity of sensing channels 83 and/or 85 to make them more sensitive to sensing low amplitude R-waves and/or fibrillation waves. When the NID is reached after increasing the sensitivity, control circuit 80 may transition to the concerned state 2 of block 104 (arrow 103a).

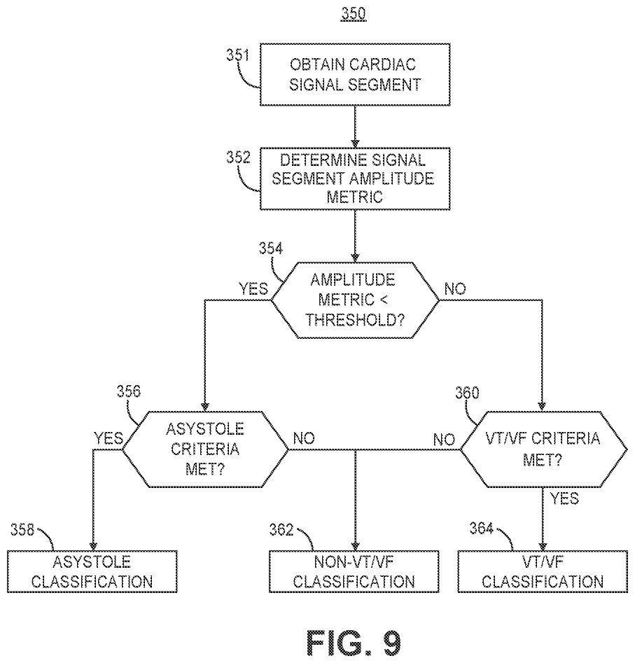

In various examples, control circuit 80 may be configured perform GMA to classify cardiac signal segments, e.g., as described in conjunction with FIG. 9 below, and determine RRIs from the Vsense signals received from sensing circuit 86 for counting VT/VF intervals for determining when criteria are met for transitioning from the unconcerned state 1 (block 102) to concerned state 2 (block 104). For instance, in other examples, when a threshold number of cardiac signal segments are classified as VT/VF out of Y consecutive cardiac signal segments (where X may equal Y in some examples), ICD 14 may transition to the concerned state 2 at block 104. For example, when at least two consecutive, two out of three, three consecutive, three out of four or other selected number of most recent cardiac signal segments are classified as VT/VF, control circuit 80 may transition to the concerned state 2 of block 104.

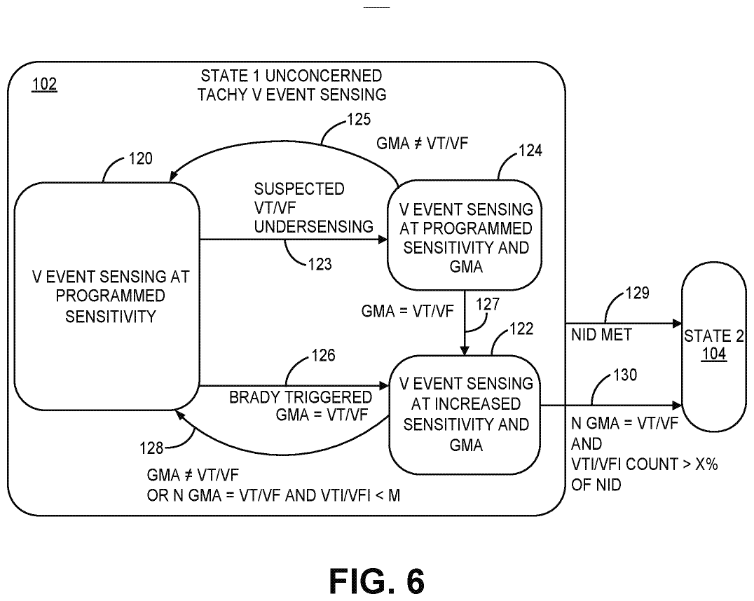

The criteria for transitioning from the unconcerned sensing state 1 of block 102 to the concerned state 2 of block 104 based on a threshold number of cardiac signal segments being classified as VT/VF based on the GMA may include requiring a threshold VTI/VFI count which may be less than the NID (but greater than zero). For instance, the VTI/VFI count may be required to be at least 3, 5, 8, 10 or 20 or other selected threshold value when at least one cardiac signal segment is classified as being VT/VF based on the GMA in order to transition to the concerned state 2 of block 104. In still other examples, multiple conditions for transitioning from the unconcerned sensing state 1 of block 102 to the concerned state 2 of block 104 may be defined that include different threshold numbers of VT/VF segments classified based on the GMA paired with different threshold values of VTI/VFI counts. For instance, if a relatively higher number of cardiac signal segments are classified as VT/VF based on the GMA, e.g., two out of two or three out of three, a relatively lower threshold value of the VTI/VFI interval counter(s) may be required, e.g., 10 VT/VF intervals out of 30 RRIs. If a relatively lower number of cardiac signal segments are classified as VT/VF based on the GMA, e.g., one out of three, a relatively higher threshold value of the VTI/VFI counts may be required, e.g., 20 out of 30 RRIs. In some examples, a threshold number of cardiac signal segments being classified as VT/VF based on the GMA when all VTI/VFI counts are zero may cause a transition from the unconcerned sensing state 1 to the concerned sensing state 2. For instance, if three, four or other threshold number of consecutive signal segments are classified as VT/VF based on the GMA, control circuit 80 may transition to the concerned sensing state 2. As such multiple state transition conditions that include an NID being reached, a threshold number of VT/VF cardiac signal segments based on the GMA, and/or one or more combinations of a threshold number of VT/VF cardiac signal segments based on the GMA and scaled VTI/VFI count values may be defined. When any one state transition condition is satisfied, control circuit 80 may transition from the unconcerned sensing state 1 of block 102 to the concerned state 2 of block 104. Aspects of the operation of ICD 14 in the tachyarrhythmia operating state of block 102, i.e., the unconcerned sensing state 1 of block 102, are described below in conjunction with FIG. 6.

It is to be understood that a transition from state 1 of block 102 to state 2 of block 104 may not be representative of a VT/VF detection that would cause ATP and/or CV/DF shock therapy to be scheduled or initiated. During the concerned tachyarrhythmia detection state 2 of block 104, control circuit 80 may suspect that a VT/VF episode may be occurring based on the NID being reached and/or other criteria being met to cause the transition to state 2 to occur. However, while operating in the concerned state 2, control circuit 80 may determine if all criteria for detecting VT/VF are met. For example, control circuit 80 may determine if any VT/VF rejection rules are met. A VT/VF rejection rule is met when processing and analysis of cardiac electrical signals sensed by sensing circuit 86 indicates that the NID may be reached due to an SVT, oversensing of cardiac events such as P-waves or T-waves being falsely sensed as R-waves, or oversensing of non-cardiac noise being falsely sensed as R-waves. A VT/VF rejection rule is applied for withholding a VT/VF detection when the likelihood or probability of the NID being reached due to a true VT/VF is uncertain or lowered based on the VT/VF rejection rule analysis of the sensed cardiac electrical signals. If no VT/VF rejection rules (also referred to herein as “rejection rules”) are met and the NID is met for detecting VT/VF, control circuit 80 may transition to the charging state 3 of block 106. VT/VF may be detected based on the NID being reached and no VT/VF rejection rules being met.

As further described below, in applying a rejection rule to withhold a VT/VF detection when an NID is met, control circuit 80 may further require that a cardiac signal segment is not classified as a VT/VF segment based on the GMA. When a cardiac signal segment is classified as a VT/VF segment, a rejection rule that is met may be overridden by the VT/VF classification of the cardiac signal segment based on the GMA such that a VT/VF detection is not withheld based on the rejection rule. In some examples, a GMA result of VT/VF classification of a cardiac signal segment may prevent a rejection rule from becoming met. Either way, when an NID is met, a GMA result of VT/VF may override withholding of a VT/VF detection based on a rejection rule.

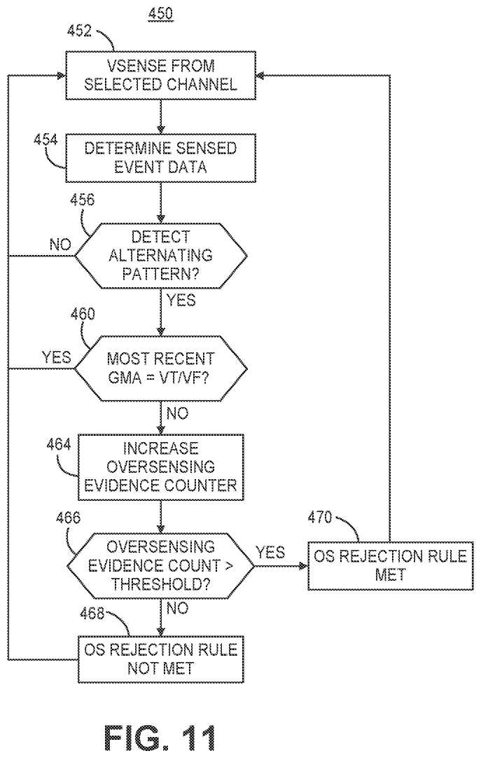

If a VT/VF rejection rule is determined to be met during the concerned state 2 of block 104 and the VT/VF rejection rule is not overridden by a VT/VF classification of a cardiac signal segment based on the GMA, ICD 14 may withhold detection of the suspected VT/VF episode and remain in the concerned state 2 of block 104 until no VT/VF rejection rules are met based on continued analysis of cardiac signals sensed by sensing circuit 86. Methods performed by ICD control circuitry during the concerned tachyarrhythmia operating state 2 of block 104 are described below, e.g., in conjunction with the flow chart of FIG. 11. Control circuit 80 may subsequently transition to the charging state 3 of block 106 when VT/VF is detected based on the NID being met when no VT/VF rejection rules are met. During charging state 3 of block 106, therapy delivery circuit 84 may begin charging a high voltage capacitor, under the control of control circuit 80, for preparing to deliver a CV/DF shock. If a cardiac signal segment is being buffered or processed for GMA upon transitioning to the charging state 3, the buffering or processing according to the GMA may continue without interruption so that a GMA result can be available for determining that abort therapy criteria are met and/or, if abort therapy criteria are met, the GMA result can be available for use in redetecting the VT/VF in redetection state 5 as described below.

In some instances, however, while operating in state 2 of block 104, control circuit 80 may determine that termination criteria are met based on analysis of the cardiac electrical signals sensed by sensing circuit 86 before all VT/VF detection criteria are satisfied (e.g., NID being met when all VT/VF rejection rules are unmet) during the concerned state 2 of block 104. Examples of termination criteria may include requiring a threshold number of cardiac signal segments classified as being non-VT/VF or asystole (not VT/VF) based on the GMA. Other termination criteria may be based on a median RRI determined from Vsense signals received from sensing circuit 86 being greater than a threshold interval. Other termination criteria may include detecting one or more normal sinus rhythm events based on an RRI and R-wave morphology matching, for example, which may cause the VTI/VFI counters to be reset, e.g., to zero or to a lower value than the current counter value.

In yet another example, termination may be detected by control circuit 80 during the concerned sensing state of block 104 when a threshold number (e.g., 6, 8, 10, 12 or other specified number) of RRIs are greater than a slow interval threshold. The slow interval threshold may be set based on the VT detection interval threshold or the VF detection interval threshold (e.g., an offset or percentage longer than a VT/VF detection interval threshold). The slow interval threshold may be determined based on the detected rhythm cycle length (RCL). For instance, the RCL may be determined as the mean or median of a specified number of most recent RRIs counted as VT/VF intervals, e.g., just prior to the NID being reached. The RCL may be computed by control circuit 80 as a trimmed mean, e.g., by determining the mean RRI after dropping the shortest and/or longest RRI of the most recent N RRIs up to the NID being met. In an example, if eight RRIs after the NID being reached are longer than the RCL plus an offset (e.g., plus 40, 50, 60, 70, or 80 ms), control circuit 80 may detect termination. A variety of methods may be implemented for determining that a suspected or detected VT/VF episode has terminated prior to therapy being delivered. Control circuit 80 may transition from the concerned state 2 of block 104 back to the unconcerned sensing state 1 of block 102 in response to termination criteria being met. Upon returning to the unconcerned sensing state 1, control circuit 206 may reset the VTI/VFI counters to zero.

During the charging state 3 of block 106, control circuit 80 may determine that abort therapy criteria are met based on an analysis of the cardiac electrical signals sensed by sensing circuit 86. Control circuit 80 may abort a CV/DF shock prior to delivery when 60%, 70%, 80% or other threshold number of a RRIs are longer than a slow interval threshold. For instance, if 4 out of the most recent 5 RRIs are at least 60 ms (or other offset) longer than the RCL, control circuit 80 may abort a CV/DF shock therapy. In another example, abort therapy criteria may be met when at least 4 out of 5 most recent RRIs are equal to or greater than the VF detection interval threshold plus an offset (e.g., 60 ms). In some examples, when VT is detected, the slow interval threshold is the RCL plus 60 ms. When the detected tachyarrhythmia is a fast VT or VF, the slow interval threshold may be the VF detection interval threshold plus 60 ms or the RCL plus 60 ms, whichever is greater. However, in some examples, when the difference between the minimum RRI and the maximum RRI used to compute the RCL is more than a threshold difference (e.g., more than 50 ms difference), the slow interval threshold may be determined as the VF detection interval threshold plus an offset, e.g., 60 ms. In still other examples, a cardiac electrical signal segment classified as VT/VF based on the GMA may satisfy abort therapy criteria. It is recognized that a variety of abort therapy criteria may be defined and applied to RRIs by control circuit 80 up until ATP or CV/DF shock delivery for making a determination that the rhythm is a slowing rhythm, justifying aborting the ATP or CV/DF shock therapy.

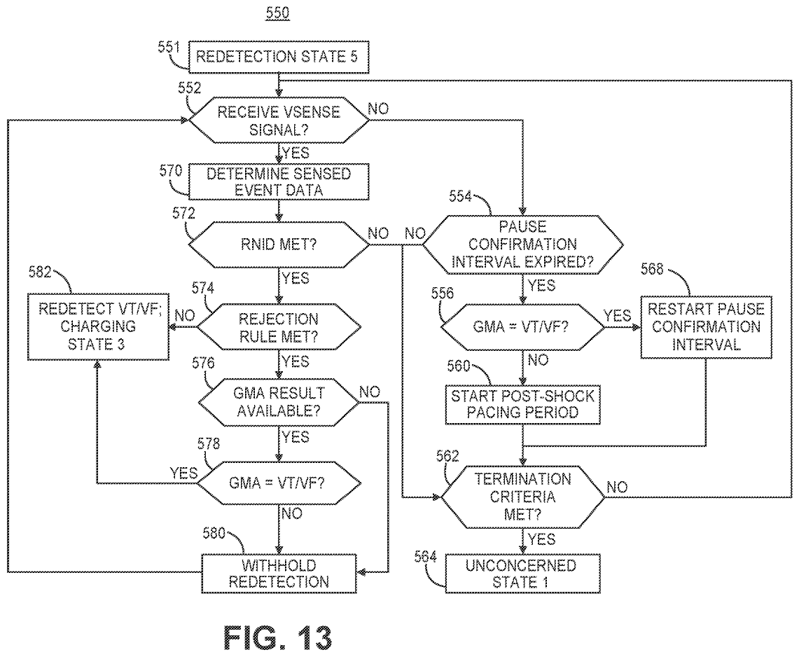

When abort therapy criteria are met during charging state 3 of block 106, control circuit 80 may terminate charging of the high voltage capacitor by therapy delivery circuit 84 (if charging is not already complete), cancel the pending CV/DF shock and advance to the redetection state 5 of block 110. Operations during the redetection state 5 are further described below. If a cardiac signal segment is being buffered or processed for GMA upon transitioning to the charging state 3 of block 106, the GMA may continue without interruption. The GMA result can be available for determining that abort therapy criteria are met and/or used by control circuit 80 during redetection state 5 if the therapy is aborted, e.g., as further described below in conjunction with FIG. 13.

In some examples, therapy delivery circuit 84 may be configured to deliver one or more sequences of ATP therapy during capacitor charging of charging state 3 (block 106). Control circuit 80 may determine that abort therapy criteria are met during capacitor charging after ATP has been delivered and advance to the redetection state 5 of block 110. In other examples, control circuit 80 may determine that termination criteria are met, which may be after delivering ATP, during or upon completion of capacitor charging. While not shown in FIG. 5, in some examples control circuit 80 may transition back to the unconcerned sensing state 1 of block 102 in response to termination criteria being met during charging state 3. The detected VT/VF may spontaneously terminate or a delivered ATP therapy may terminate the VT/VF episode without having to deliver a CV/DF shock.

When capacitor charging is completed for delivering a therapy during charging state 3 (block 106) before abort therapy criteria are met, control circuit 80 may transition to the therapy delivery state 4 of block 108. Therapy delivery circuit 84 may deliver ATP or a CV/DF shock after the transition to state 4. Therapy delivery circuit 84 may be controlled by control circuit 80 to synchronize the ATP or the CV/DF shock to the patient's intrinsic heart rhythm, e.g., based on the timing of received Vsense signals. The therapy may be synchronized to a Vsense signal received outside a refractory period. The therapy may be delivered upon expiration of a specified time interval after charge completion. In other examples, a CV/DF shock can be delivered during state 4 without necessarily being synchronized to the patient's intrinsic heart rhythm. In some cases, abort therapy criteria could be met after capacitor charging is completed but before the therapy is delivered, e.g., while waiting for a synchronizing event during the therapy delivery state 4.

Control circuit 80 may determine that abort therapy criteria are met after capacitor charging is completed when a specified number of Vsense signals are refractory events (e.g., when three or another specified number of consecutive Vsense signals are received during a post-sense ventricular refractory period). Control circuit 80 may determine that abort therapy criteria are met after capacitor charging when a single RRI is greater than the slow interval threshold or when any of the other examples of abort therapy criteria described above are met. It is to be understood that any time after capacitor charging has started, when abort therapy criteria are met before the therapy is delivered, control circuit 80 may transition to redetection state 5 of block 110 without delivering the therapy.

After a therapy is delivered in state 4 of block 108, which may be ATP or a CV/DF shock (or when abort therapy criteria are met before therapy delivery), control circuit 80 may transition to redetection state 5 at block 110. VT/VF may be redetected by control circuit 80 when a reduced NID, which may be referred to as a “redetection NID” or “RNID,” is met following therapy delivery or after a therapy is aborted due to the abort therapy criteria being met. Control circuit 80 may determine that the RNID is met based on Vsense signals received from the sensing channel 83 or 85 that was selected as the reliable sensing channel for tachyarrhythmia detection when control circuit 80 transitioned to the concerned state 2. In some examples, control circuit 80 may redetect VT/VF when the RNID is met and any VT/VF rejection rules applied during redetection state 5 are not met. The RNID may be, for example, 25%, 30%, 50%, 60%, or 70% of the NID required to transition from the unconcerned state 1 to the concerned state 2. The RNID may be used by control circuit 80 for redetecting VT/VF when the probability of VT/VF is relatively high due to the previous known VT/VF detection made in concerned state 2. If VT/VF redetection criteria are met in state 5, control circuit 80 may return to the charging state 3 of block 106. Methods that may be performed by control circuit 80 during the redetection operating state 5 of block 110 are described below, e.g., in conjunction with the flow chart of FIG. 13.

A CV/DF shock can be delivered (one or more times) by therapy delivery circuit 84 to terminate the VT/VF. Control circuit 80 may be configured to apply redetection criteria and/or termination criteria to cardiac electrical signals sensed by sensing circuit 86 after each CV/DF shock (during redetection state 5) for determining if the detected VT/VF has been terminated or an additional shock is needed based on redetection criteria being met. If termination criteria are met during the redetection state 5 before redetection criteria are met, control circuit 80 may transition back to the unconcerned sensing state 1 of block 102. The VTI/VFI counters may be reset to zero, and GMA may be disabled upon transitioning to the unconcerned sensing state 1.

When control circuit 80 transitions back to the unconcerned sensing state 1 of block 102, e.g., from the concerned state 2 of block 104 or from the redetection state 5 of block 110 when termination criteria are met, control circuit 80 may restore the sensitivity of the sensing channel(s) 83 and 85 to the programmed sensitivity (if the sensitivity had previously been increased by setting a lower voltage amplitude used as the R-wave sensing floor in response to a cardiac signal segment being classified as VT/VF based on the GMA). When the sensitivity has been increased in the unconcerned state 1 of block 102 and criteria are subsequently met for transitioning to the concerned state 2 of block 104 with the increased sensitivity in effect, the increased sensitivity may remain in effect during all subsequently reached tachyarrhythmia operating states of blocks 104, 106, 108 and 110 (during the therapy delivery in state 4 of block 108 sensing circuit 86 may be blanked or disabled). The increased sensitivity of sensing circuit 86 may be applied for sensing ventricular event signals for determining when termination criteria are met (before and/or after therapy delivery), determining when abort therapy criteria are met, synchronizing a VT/VF shock, and determining when redetection criteria are met after therapy delivery, for example. Each time ICD 14 enters the unconcerned sensing state 1 of block 102, the sensitivity for sensing channels 83 and 85 may be restored to the programmed sensitivity, e.g., to the user programmed setting or a default sensitivity setting.

FIG. 6 is a conceptual diagram 200 of the unconcerned sensing state 1 of the tachyarrhythmia operating states shown in FIG. 5. Sensing methods performed by ICD 14 for detecting VT/VF, detecting termination of VT/VF, and redetecting VT/VF may generally be referred to as tachyarrhythmia sensing methods. Sensing methods performed by ICD 14 for determining a need for bradycardia pacing, e.g., for detecting a long pause in ventricular activity, may generally be referred to as bradycardia sensing methods. Tachyarrhythmia sensing methods and bradycardia sensing methods may include interactions that can affect the operations being performed during the tachyarrhythmia operating states and during the bradycardia operating states.

Referring to FIG. 6, at block 120 of the unconcerned sensing state 1 of the tachyarrhythmia sensing methods, each sensing channel 83 and 85 of sensing circuit 86 may be sensing ventricular event signals, e.g., R-waves, according to a programmed sensitivity. Control circuit 80 may determine RRIs between consecutively received Vsense signals received from a given sensing channel for counting VTIs and VFIs in a VTI counter and VFI counter, respectively, (and in some examples a combined VT/VF interval counter) designated for each respective sensing channel.

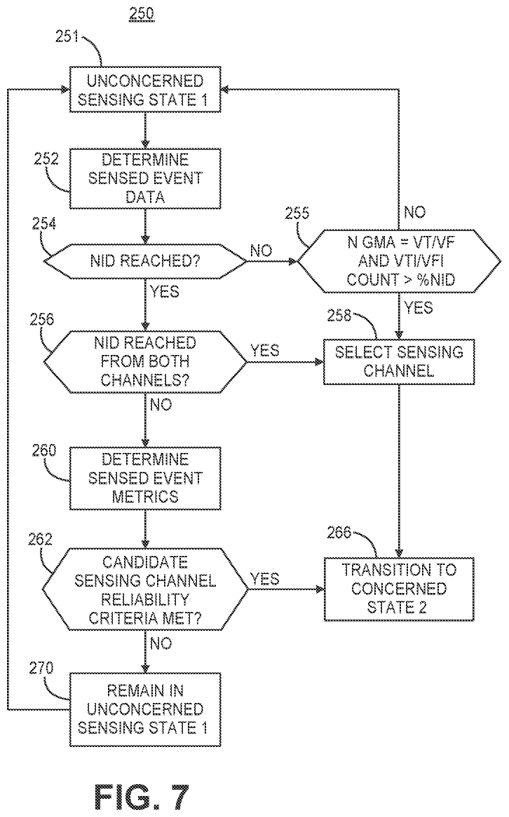

As further described below in conjunction with FIG. 7, when an NID is reached by one sensing channel 83 or 85, based on RRIs determined from Vsense signals received from that sensing channel, control circuit 80 may analyze sensed event data determined for Vsense signals received from both sensing channels 83 and 85 to select one of sensing channels 83 or 85 as a reliable sensing channel for VT/VF detection. If the selected sensing channel 83 or 85 is the sensing channel that has reached the NID, control circuit 80 may transition to the concerned state 2 of block 104 (as indicated by arrow 129). The selected sensing channel is used for sensing ventricular event signals (and generating corresponding Vsense signals) upon transitioning to the concerned state 2 of block 104. Control circuit 80 may use the Vsense signals received from the selected sensing channel for the tachyarrhythmia sensing methods for detecting VT/VF, including updating the VTI and VFI counters, detecting termination, detecting an abort therapy condition, redetecting VT/VF and any other operations that require ventricular event signal sensing such as determining RRIs and triggering a sensed event signal segment to be stored for R-wave morphology matching or other sensed event signal analysis as further described below.

If the selected sensing channel is not the sensing channel that has reached the NID, however, control circuit 80 may remain in the unconcerned state 1 of block 102. Control circuit 80 may continue to use Vsense signals received from both sensing channels 83 and 85 for counting VTIs and VFIs for each respective sensing channel. When an NID is reached by a VTI or VFI counter for both sensing channels 83 and 85, control circuit 80 may transition to the concerned state 2 of block 104 (as indicated by arrow 129).

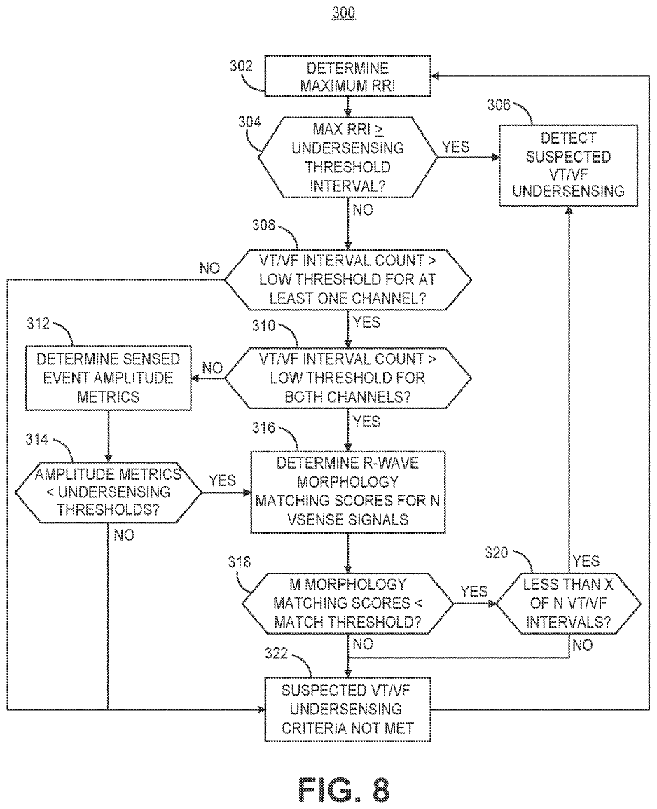

While operating in the unconcerned state 1 of block 102, sensing channels 83 and 85 may sense ventricular event signals based on the programmed sensitivity. During ventricular event sensing at the programmed sensitivity (block 120), control circuit 80 may determine when suspected VT/VF undersensing (also referred to herein as “suspected tachyarrhythmia undersensing”) criteria are met based on the ventricular event sensing. Methods for detecting suspected VT/VF undersensing are described below in conjunction with FIG. 8. In one example, when an RRI determined from Vsense signals received from either sensing channel 83 or 85 is greater than an undersensing threshold, control circuit 80 may detect suspected VT/VF undersensing. Other examples of criteria for detecting suspected VT/VF undersensing are described below in conjunction with FIG. 8.

When suspected VT/VF undersensing is detected (as indicated by arrow 123), control circuit 80 may trigger a GMA of a cardiac signal segment at block 124. In some examples, morphology sensing channel 87 may be powered down or disabled until GMA is triggered by control circuit 80, e.g., to conserve power source 98, when no other morphology signal analyses are required by control circuit 80 for tachyarrhythmia or bradycardia sensing methods. In other examples, cardiac signal segments may be buffered in memory 82 from morphology signal channel 87 but not processed and analyzed by control circuit 80 according to the GMA until the GMA is triggered, e.g., based on detecting suspected VT/VF undersensing. If GMA is not triggered, a buffered cardiac signal segment may be discarded or overwritten by a new cardiac signal segment, e.g., on a first-in-first-out basis.

When GMA is triggered, control circuit 80 may buffer an n-second cardiac signal segment in memory 82 for processing and analysis for determining if morphological evidence of VT/VF is present in the cardiac signal segment. Based on the GMA (block 124), control circuit 80 may classify the cardiac signal segment as a VT/VF segment, a non-VT/VF segment or an asystole segment. Examples of GMA methods that may be performed by control circuit 80 for classifying a cardiac signal segment as VT/VF, non-VT/VF or asystole are described below in conjunction with FIG. 9.

When the cardiac signal segment is not classified as VT/VF (e.g., classified as asystole or non-VT/VF), control circuit 80 may return to block 120 (as indicated by arrow 125). Sensing channels 83 and 85 may continue to sense ventricular event signals according to the programmed sensitivity and updating VTI and VFI counters as RRIs are determined between Vsense signals and fall in a respective VT detection interval zone or a VF detection interval zone.

Referring again to block 124, when a cardiac signal segment is classified as VT/VF based on the GMA performed at block 124, sensing circuit 86 may advance to block 122 (as indicted by arrow 127) and may increase the sensitivity of sensing channel 83 and/or sensing channel 85 for sensing ventricular event signals. In some examples, the sensitivity of both sensing channels 83 and 85 is increased by decreasing the voltage amplitude of the sensitivity setting, e.g., to lower the sensing floor. The sensitivity of both sensing channels 83 and 85 may be increased to a maximum sensitivity, e.g., to the minimum available voltage amplitude setting that defines the sensing floor. For the sake of convenience, illustrative examples described herein refer to both sensing channels 83 and 85 being adjusted to control the R-wave sensing threshold according to a maximum sensitivity in response to the GMA resulting in a VT/VF classification of the cardiac signal segment. It is noted that the “maximum sensitivity” refers to the lowest voltage amplitude setting available for sensing circuit 86, which may be in a range of tens of microvolts to millivolts (mV) in various examples. For instance, the sensitivity may be programmable in a range of 0.03 to 2.0 mV or between 0.075 and 1.2 mV, as examples. The maximum sensitivity, therefore, may be a voltage amplitude of 0.03, 0.05, 0.075, 0.1, or 0.15 mV, in various examples, and can be any minimum voltage amplitude setting that is available for sensing circuit 86 to use as the sensing floor for sensing ventricular event signals.

It is to be understood, however, that in other examples sensing circuit 86 may increase the sensitivity of one sensing channel 83 or sensing channel 85 at block 122, but not necessarily both, in response to the cardiac signal segment being classified as a VT/VF segment. In still other examples, sensing circuit 86 may increase the sensitivity of one or both sensing channels 83 and/or 85 toward a maximum sensitivity (e.g., toward the minimum available voltage amplitude setting for sensitivity) but not necessarily to the maximum sensitivity. For instance, sensing circuit 86 may adjust the sensitivity to be one-half or other fraction or percentage of the current voltage amplitude setting for the programmed sensitivity or may decrease the voltage amplitude setting by a specified decrement or to the next lower voltage amplitude setting available in various examples.

If an NID is reached based on ventricular event signals received from at least one sensing channel 83 or 85 after increasing the sensitivity, and that channel is selected as the reliable sensing channel for VT/VF detection (as described in conjunction with FIG. 7 below), control circuit 80 may transition to the concerned state 2 of block 104 (as indicated by arrow 129). By increasing the sensitivity, R-waves or fibrillation waves that may have been undersensed during a VT/VF episode, as evidenced by the GMA result, may now be sensed using the lower sensing floor thereby enabling the VTI and VFI counters to properly track VTIs and VFIs and reach a corresponding NID. As such, when an NID is met by a VTI or VFI counter for one or both sensing channels, the sensitivity that is in effect may be the programmed sensitivity or an increased sensitivity. The sensitivity in effect at the time that the NID is reached may remain in effect, for use in sensing ventricular event signals by the selected sensing channel 83 or 85, after transitioning to the concerned state 2 of block 104. The sensitivity in effect at the time of the transition to state 2 may remain in effect until control circuit 80 transitions back to the unconcerned state 1 of block 102 from any of tachyarrhythmia operating states 2, 3 or 5 (according to the methods described above in conjunction with FIG. 5).

Referring again to blocks 124 and 122, if one GMA results in a VT/VF classification of a cardiac signal segment at block 124 (e.g., as indicated by arrow 127), control circuit 80 may continue to perform the GMA for at least one or more subsequent cardiac signal segments at block 122. One or more cardiac signal segments consecutively received from morphology sensing channel 87 can be buffered in memory 82 for processing and analysis by control circuit 80 at block 122 of the unconcerned sensing state 1. The GMA may continue to be performed by control circuit 80 at block 122 until at least one cardiac signal segment (or another specified number of cardiac signal segments) is not classified as VT/VF (e.g., classified as asystole or non-VT/VF). Control circuit 80 may return to block 120 and restore the programmed sensitivity of sensing channel 86 if a cardiac signal segment is classified as asystole or non-VT/VF, for example, as shown by arrow 128.

In some examples, the GMA is performed a maximum number of times, e.g., by analyzing a maximum number of cardiac signal segments. In an example, GMA may be performed at block 122 on up to a maximum of three cardiac signal segments (which may correspond to about 9 to 12 seconds of operating at the increased sensitivity in block 122). If a cardiac signal segment is not classified as VT/VF, control circuit 80 returns to block 120. If three consecutive cardiac signal segments are classified as VT/VF, control circuit 80 may make a decision whether to transition to block 120 and restore the programmed sensitivity setting or transition to state 2 based on the status of the VTI/VFI counters.

When a threshold number of cardiac signal segments are classified as VT/VF based on the GMA, control circuit 80 may transition to the concerned state 2 of block 104 (as indicated by arrow 130) before an NID is reached by either sensing channel 83 or 85 in some examples. For instance, when a threshold number of consecutive cardiac signal segments are classified as VT/VF based on the GMA and at least one of the VTI counter or the VFI counter for a sensing channel 83 or 85 has reached at least a specified percentage of the respective NID, control circuit 80 may transition to the concerned state 2 of block 104 (arrow 130). In an illustrative example, if the NID for VF detection is 30 VFIs out of 40 RRIs and at least 20 VFIs have been counted for one sensing channel 83 or 85, control circuit 80 may transition to the concerned state 2 of block 104 when at least three consecutive cardiac signal segments are classified as VT/VF. The sensing channel 83 or 85 having a VTI/VFI counter meeting the threshold number of VTIs or VFIs may be selected as the reliable sensing channel for use in detecting VT/VF after transitioning to the concerned state 2.

Other criteria may be conceived for forcing a transition to the concerned state 2 before an NID is met but sufficient evidence for a suspected VT/VF episode is detected based on one or more cardiac signal segments being classified as VT/VF according to the GMA along with an increasing trend of the VTI and/or VFI counters for at least one sensing channel 83 or 85. For example, if three cardiac signal segments are classified as VT/VF and a majority of the most recent RRIs are counted as VTIs or VFIs, control circuit 80 may transition to state 2 of block 104. In various examples, if at least two, three or other threshold number of consecutive cardiac signal segments are classified as VT/VF based on the GMA and a VTI/VFI counter has reached at least a threshold percentage of the NID and/or at least a threshold percentage (e.g., 60%, 75%, 80%, or 100%) of a specified number of recent RRIs, e.g., most recent 8, 10, or 12 RRIs, are VTIs and/or VFIs for at least one sensing channel 83 or 85, control circuit 80 may force a transition to the concerned state 2 of block 104 prior to an NID to be met.

After control circuit 80 transitions to concerned state 2 of block 104, the GMA may continue to be enabled until control circuit 80 transitions back to the unconcerned sensing state of block 102. Control circuit 80 may continue to buffer cardiac signal segments in memory 82 and perform the GMA on the cardiac signal segments in any of the tachyarrhythmia operating states 2, 3 or 5 described above in conjunction with FIG. 5.

Referring again to block 122, if an NID is not met and a threshold number of cardiac signal segments have been analyzed and are classified as VT/VF, control circuit 80 may return to block 120 (as indicated by arrow 128 when the criteria N GMA=VT/VF and VTI/VFI<M, where M may equal the NID or a specified percentage of the NID in various examples). If the VTI/VFI counters are not trending upward, e.g., have not reached a specified percentage of the NID when the threshold number of cardiac signal segments are classified as VT/VF (as indicated by arrow 130), control circuit 80 may return to block 120 (as indicated by arrow 128). For example, if all VTI/VFI counters are zero, less than five, less than ten, or less than twenty (or other specified threshold value which may be less than or equal to the NID in various examples) and the maximum number of consecutive cardiac signal segments have been analyzed (and classified as VT/VF), control circuit 80 may return to block 120. It may be assumed that if a true VT/VF episode is occurring, an NID would be reached by at least one of the sensing channels 83 or 85 within the threshold number of cardiac signal segments after increasing the sensitivity of sensing channels 83 and 85 for sensing ventricular event signals.

Upon transitioning back to block 120, control circuit 80 may restore the sensitivity of the sensing channels 83 and 85 to their programmed values. Control circuit 80 may discontinue buffering and/or performing GMA of cardiac signal segments (if GMA is not currently triggered by bradycardia sensing methods as described below). GMA may be discontinued to conserve power source 98 and reduce processing burden.

In some examples, control circuit 80 may trigger a GMA of a cardiac signal segment during bradycardia sensing methods when a suspected long pause (in ventricular activity) is detected. A “long pause” as used herein refers to a minimum specified time interval during which no ventricular activity is detected according to the bradycardia sensing methods disclosed herein. A long pause may occur when no ventricular activity is sensed for at least 3 to 9 seconds, for example. Ventricular activity may be detected based on Vsense signals received from one or both sensing channels 83 and/or 85 and/or based on the GMA of a cardiac signal segment. A GMA triggered by bradycardia sensing methods for detecting or confirming a long pause may affect the tachyarrhythmia sensing methods in some examples. As shown in block 102 by arrow 126, for example, control circuit 80 may advance to block 122 from block 120 when a GMA triggered by bradycardia sensing methods results in a VT/VF classification of the analyzed cardiac signal segment.

For example, when control circuit 80 is operating in block 120 of the unconcerned tachyarrhythmia operating state, ventricular event signal sensing is performed using the programmed sensitivity and Vsense signals can be received by control circuit 80 from both sensing channels 83 and 85 for use in bradycardia sensing methods and tachyarrhythmia sensing methods. Control circuit 80 may perform a first analysis of Vsense signals received from sensing circuit 86 for detecting a suspected long pause during a pause detection interval. Control circuit 80 may perform a second analysis of Vsense signals received from sensing circuit 86 for detecting suspected VT/VF undersensing at block 120 of the concerned state 1 of the tachyarrhythmia operating states. If the first analysis results in a suspected long pause detection or if the second analysis results in determining suspected VT/VF undersensing, control circuit 80 may trigger the GMA to be performed for either confirming the long pause or confirming the likelihood of VT/VF undersensing, respectively.

The first analysis for detecting a suspected long pause may include determining if any Vsense signals are received during a long pause detection interval (which may be 2 to 3 seconds long as examples). Generally, if a Vsense signal is received, control circuit 80 may evaluate the peak amplitude of the cardiac electrical signal sensed by one or both sensing channels 83 and 85, e.g., during the post-sense blanking period, for determining if the Vsense signal can be trusted. Control circuit 80 may detect a suspected long pause when no trusted Vsense signals are received for a long pause detection interval. As such, as shown in block 102 by arrow 126, sensing circuit 86 may be operating according to the programmed sensitivity in block 120 during the unconcerned state 1 when a cardiac signal segment is classified as VT/VF based on the GMA triggered by bradycardia sensing methods. Control circuit 80 may advance to block 122. Example methods that may be performed by control circuit 80 for triggering a GMA according to bradycardia sensing methods and tachyarrhythmia sensing methods are generally described in provisional U.S. Patent Application No. 63/486,725 (Greenhut, et al., filed Feb. 24, 2023), incorporated herein by reference in its entirety.

Sensing circuit 86 may increase the sensitivity of sensing channels 83 and 85 at block 122, according to any of the examples described above. Control circuit 80 may perform at least one additional GMA of a subsequent cardiac electrical signal segment at block 122. In some examples, control circuit 80 may additionally require that all VTI/VFI counters for both sensing channels be at a value that is less than a threshold value in order to advance to block 122 from block 120 in response to a cardiac signal segment being classified as a VT/VF segment when bradycardia sensing methods trigger the GMA. For instance, control circuit 80 may require that all VTI/VFI counters be at 0 or less than 3, 5, 8, 10, 12, 15, 20 or other threshold value in order to advance to block 122 and increase the sensitivity of sensing circuit 86. When a VTI/VFI counter is greater than the threshold value, indicating Vsense signals are being received at VTIs and/or VFIs and counted as such, undersensing of VT/VF may not be occurring. An increased sensitivity may not be warranted. However, when the VTI/VFI counters are all less than a threshold value and a VT/VF classification of a cardiac signal segment is made, control circuit 80 may increase the sensitivity by advancing to block 122 to avoid undersensing of low amplitude R-waves and fibrillation waves that may be occurring at VT/VF intervals that are not being counted.

Control circuit 80 may continue to perform the GMA of cardiac signal segments received from the morphology signal channel 87 at block 122 until any of the conditions for transitioning to the concerned state 2 are met or until at least one cardiac signal segment is not classified as VT/VF (arrow 128) or a threshold number of VT/VF classifications are made based on the GMA but the NID is not trending up despite the increased sensitivity of sensing channel 86 according to any of the examples described above. When control circuit 80 is operating in block 122 with sensing circuit 86 operating according to the increased sensitivity, Vsense signals received from sensing channels 83 and 85 generated in response to sensing ventricular event signals according to the increased sensitivity can be used by control circuit 80 for both tachyarrhythmia sensing methods and bradycardia sensing methods. In this way, control circuit 80 may increase the sensitivity of sensing channels 83 and/or 85 for tachyarrhythmia sensing based on bradycardia sensing methods being performed for detecting a long pause and a need for bradycardia pacing. Conversely, control circuit 80 may increase the sensitivity of sensing channels 83 and/or 85 for bradycardia sensing based on tachyarrhythmia sensing methods being performed for detecting suspected VT/VF undersensing and a need for a CV/DF shock.

FIG. 7 is a flow chart 250 of a method that may be performed by control circuit 80 for selecting a reliable sensing channel for detecting VT/VF according to some examples. A reliable sensing channel may be selected by control circuit 80 in response to a condition being met for transitioning to the concerned state 2 of block 104 of FIG. 5.

At block 251, control circuit 80 is operating in the unconcerned sensing state 1 (block 102 of FIG. 6). Control circuit 80 receives Vsense signals from sensing circuit 86, from both sensing channel 83 and sensing channel 85, as they are being generated. In response to each Vsense signal, sensing circuit 86 and control circuit 80 may cooperatively determine sensed event data at block 252. The sensed event data can be buffered in memory 82 for analysis for selecting a reliable sensing channel for detecting VT/VF, e.g., when an NID is reached based on Vsense signals received from one of the sensing channels 83 or 85.

Sensing circuit 86 and control circuit 80 may cooperatively determine the sensed event data at block 252 by determining a matched or unmatched sensed event classification, a noise metric, the sensed event peak amplitude, the R-wave sensing threshold amplitude at the time of the R-wave sensing threshold crossing that resulted in the Vsense signal being generated, and the RRI. Control circuit 80 may determine the RRI as the time interval from the currently received Vsense signal to the most recent preceding in-channel Vsense signal, received from the same sensing channel 83 or 85 (or in some instances from a preceding pacing pulse). The RRI, also referred to herein as a “ventricular sensed event interval,” may be buffered in memory 82.

In response to the Vsense signal, sensing circuit 86 may start an in-channel blanking period that is applied to the received cardiac electrical signal by the respective sensing channel 83 or 85, e.g., to avoid sensing the same signal twice. The maximum peak amplitude of the sensed signal may be determined during the in-channel blanking period, e.g., by a peak track and hold circuit of the R-wave detector 66a or 66b (shown in FIG. 4) of the respective sensing channel 83 or 85. The maximum peak amplitude may be buffered in memory 82 in conjunction with the RRI at block 152 as sensed event data corresponding to the Vsense signal.

Control circuit 80 may determine an amplitude to sense threshold ratio (ASTR) at block 252 as sensed event data for each Vsense signal. The ASTR can be determined by determining the ratio of the maximum peak amplitude determined during the in-channel blanking period to the amplitude of the R-wave sensing threshold at the time that the cardiac electrical signal crossed the R-wave sensing threshold resulting in the Vsense signal.

A noise metric may be determined by sensing circuit 86 and/or control circuit 80 at block 252 for assessing the noisiness of the cardiac electrical signal at the time of the Vsense signal. In some examples, the noise metric can be determined as a noise pulse count by counting the number of signal pulses that cross a noise pulse threshold amplitude during the in-channel blanking period. The noise pulse threshold amplitude may be equal to or based on the R-wave sensing threshold amplitude that was crossed by the cardiac electrical signal resulting in the Vsense signal. The noise pulses may be counted by applying the noise pulse threshold to the narrowband, notch filtered and rectified signal, received during the in-channel blanking period, by the respective R-wave detector 66a or 66b. The noise pulse count of the number of signal pulses during the in-channel blanking period having an amplitude greater than or equal to the R-wave sensing threshold amplitude at the time of the Vsense signal can be representative of the noisiness of the cardiac electrical signal at the time of the Vsense signal. In other examples, the noise metric may be determined by counting a number of signal peaks, zero crossings, determining a mean amplitude, mean slope or other feature of the cardiac electrical signal during the in-channel blanking period. The noise metric may be buffered in memory 82 in association with the RRI and other sensed event data determined for the Vsense signal at block 252. In other examples, the noise metric may be compared to a threshold value or range for identifying the sensed event signal as a noisy event. The Vsense signal may be classified as a noisy or non-noisy event based on the noise metric. The noisy or non-noisy event classification may be buffered in memory 82 at block 252 with other sensed event data corresponding to the Vsense signal. For example, if the noise pulse count is at least 3, 4, 5, 6 or other specified threshold number, a noisy event classification may be stored with the sensed event data for the corresponding Vsense signal.

Control circuit 80 may determine if the current Vsense signal is a matched or unmatched event signal at block 252. The current Vsense signal is a matched signal when control circuit 80 receives a Vsense signal from the other sensing channel 83 or 85 within a specified time window of the current Vsense signal. For instance, control circuit 80 may buffer a matched event signal classification in memory 82 for the current Vsense signal when it is preceded or followed by a Vsense signal received from the other sensing channel within a matching window. The matching window may be, in one example, a 160 ms time window that may extend 80 ms earlier and 80 ms later than the current Vsense signal. If another Vsense signal is not received from the other sensing channel during the matching window, control circuit 80 may buffer an unmatched event signal classification in memory 82 at block 252 for the current Vsense signal. The matching window may be 50 to 200 ms long in various examples and may extend (equally or unequally) before and/or after the time of the current Vsense signal.

For each Vsense signal received from each sensing channel 83 and 85, control circuit 80 may determine and buffer the RRI, the peak amplitude, the ASTR, the noise metric (or the noisy event or non-noisy event classification) and the matched or unmatched event classification. This sensed event data determined for the Vsense signal may be stored in a buffer in memory 82 that is allocated for storing sensed event data for the respective sensing channel. In other examples, other features of the cardiac electrical signal received by the respective sensing channel 83 or 85 may be determined and buffered in memory 82 as sensed event data for use in determining a reliable sensing channel for VT/VF detection. For example, in addition to or alternatively to the RRI, peak amplitude, ASTR, noise metric and matched/unmatched classification, examples of other cardiac signal features that may be determined from the cardiac electrical signal received from the sensing channel 83 or 85 may include: a peak positive slope; peak negative slope; R-wave template morphology matching score; signal width of the maximum amplitude pulse following the R-wave sensing threshold crossing; maximum signal width; number of signal pulses having less than a threshold signal width; a sum of pulse widths during the blanking interval (or another baseline portion of the cardiac electrical signal); or any combination of any of these examples.

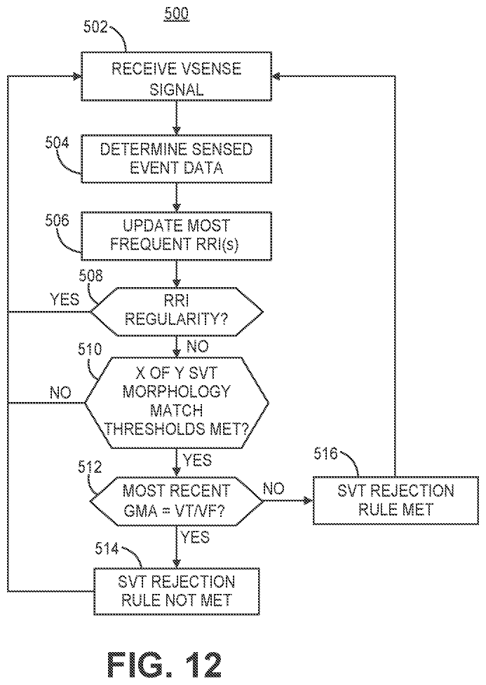

The sensed event data that is determined and stored in memory 82 by control circuit 80 may include data that is used by control circuit 80 for determining if a rejection rule for withholding a VT/VF detection is met when an NID is reached. Examples of sensed event data that may be determined for applying a rejection rule when an NID is met are described below in conjunction with FIGS. 11 and 12. The sensed event data that is determined in response to a Vsense signal may depend on the value of a VTI/VFI counter. For example, the morphology sensing channel 87 may be enabled for passing a morphology signal from which R-wave morphology matching scores can be determined as sensed event data when a VTI/VFI counter has reached at least a threshold value, e.g., 3, 5, 8 or other specified value.

It is to be understood that control circuit 80 may buffer two different types of cardiac signal segments in memory 82 for processing and analysis. Control circuit 80 may buffer sensed event signal segments for analyzing sensed event signals for determining sensed event data. The sensed event signal segments are different than the cardiac signal segments that are obtained and buffered for performing the GMA. A sensed event signal segment is buffered in response to a Vsense signal received from sensing circuit 86. The sensed event signal segments can be relatively short signal segments compared to the cardiac signal segments acquired for performing GMA. For example, the sensed event signal segments may be 500 ms or less or 350 ms or less, as examples. The sensed event signal segments are intended to encompass the signal waveform that was sensed by one of sensing channels 83 and/or 85 and caused sensing circuit 86 to generate a Vsense signal. Thus the signal waveform buffered in the sensed event signal segment corresponds to one sensed event signal and the one corresponding Vsense signal produced by a sensing channel (though both sensing channels 83 and 85 may produce a Vsense signal in response to sensing the same signal waveform). The sensed signal waveform buffered in the sensed event signal segment may be a true R-wave or fibrillation wave but can be a oversensed signal, e.g., an oversensed P-wave, T-wave, or non-cardiac noise signal. Thus, buffering of a sensed event signal segment for R-wave morphology matching (and/or other sensed event signal analysis) for use in selecting a reliable sensing channel for VT/VF detection and/or for determining if a rejection rule for withholding a VT/VF detection is met or not (e.g., as described below in conjunction with FIGS. 11 and 12) is dependent on receiving a Vsense signal from sensing channel 83 or 85. That is, a sensed event signal segment of the morphology signal received from morphology sensing channel 87 can be fetched by control circuit 80 in response to receiving a Vsense signal. The sensed event signal segment has a beginning and ending time dependent on and defined relative to the timing of the Vsense signal.

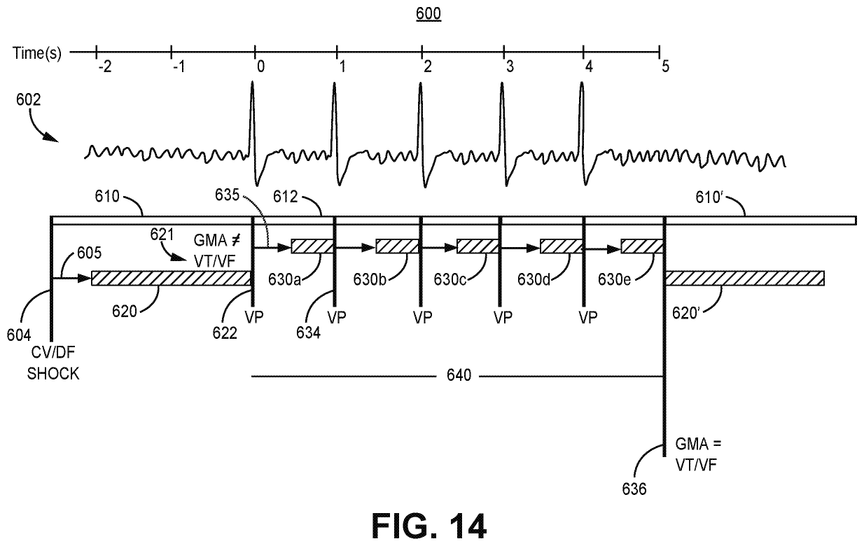

In contrast, the cardiac signal segment acquired for undergoing GMA can be relatively longer than the sensed event signal segments and can begin and end independent of the timing of any Vsense signal. The cardiac signal segment buffered for GMA can be, for example, 1 to 6 seconds long or 3 seconds in the illustrative examples presented herein. The cardiac signal segment used for GMA may have a total time duration that is greater than multiple VT or VF detection intervals, e.g., five to ten times longer than a VT or VF detection interval. The sensed event signal segment has a total time duration that encompasses a single in-channel Vsense signal whereas a cardiac signal segment buffered for GMA may have none, one or many Vsense signals occurring during the cardiac signal segment but the timing of the beginning and end of the cardiac signal segment can be independent or random relative to any Vsense signal that occurs. As described below in conjunction with FIG. 14, during bradycardia or post-shock pacing a relatively shorter cardiac signal segment may be buffered for GMA between pacing pulses, e.g., a 0.3 to 1.0 second segment so that GMA result can be determined from multiple cardiac signal segments encompassing a total time that is greater than multiple VT or VF detection intervals. The cardiac signal segments acquired for GMA may or may not include and are independent of the timing of any Vsense signals. The time interval over which a cardiac signal segment for GMA can be independent of receiving a Vsense signal because, in some instances, no Vsense signals are being received when the cardiac signal segment is being buffered. None, one or more sensed event signal segments may be buffered during the buffering of the cardiac signal segment depending on how many Vsense signals, if any, are received from sensing circuit 86 during the cardiac signal segment.

At block 254, control circuit 80 determines if an NID is reached by a VTI/VFI counter for either sensing channel 83 or 85 based on the RRIs determined for the Vsense signals received from the respective sensing channel 83 or 85. If the VTI/VFI counters for both sensing channels 83 and 85 are less than a respective NID for detecting VT or VF, control circuit 80 may remain in the unconcerned sensing state 1 by returning to block 251. However, as shown in FIG. 7 and as described above in conjunction with FIG. 6, if a threshold number, e.g., 3, cardiac signal segments are classified as VT/VF based on the GMA (decision block 255) and a VTI/VFI counter is trending upward (e.g., has reached at least a specified percentage of the NID), control circuit 80 may select a sensing channel for VT/VF detection at block 258. Control circuit 80 may transition to the concerned state 2 at block 266.

At block 258, control circuit 80 may select a sensing channel 83 or 85 in response to the N GMA results being VT/VF and at least one VTI/VFI counter reaching a threshold percentage of the NID. The selected sensing channel may be associated with the VTI/VFI counter that reached the threshold percentage of the NID. If both sensing channels 83 and 85 have reached the threshold percentage of the NID, the selected sensing channel may be a programmed default sensing channel or the sensing channel 83 or 85 that was most recently selected as being a reliable sensing channel for VT/VF detection. In other examples, the sensing channel 83 or 85 associated with the highest VTI, VFI or combined VTI and VFI count may be selected as the reliable sensing channel at block 258. The Vsense signals received from the selected sensing channel 83 or 85 can be used by control circuit 80 for adjusting the VTI/VFI counters for VT/VF detection and for detecting a long pause for initiating ventricular pacing according to bradycardia sensing methods while control circuit 80 operates in the concerned state 2 for detecting VT/VF.

Referring again to block 254, when control circuit 80 determines that an NID is reached (“yes” branch), control circuit 80 determines if the NID is reached by the VTI/VFI counters for only one sensing channel 83 or 85 or both sensing channels 83 and 85 at block 256. In some instances, VTI/VFI counters for both sensing channels 83 and 85 may reach the NID required for detecting VT/VF at about the same time, e.g., on the same matched Vsense signal. When VTI/VFI interval counters associated with both sensing channels 83 and 85 have reached an NID (“yes” branch of block 256), control circuit 80 may select a default sensing channel at block 258 and transition to the concerned state 2 at block 266. The default sensing channel may be a programmed default sensing channel or the sensing channel 83 or 85 that was most recently selected as being a reliable sensing channel for VT/VF detection (according to the methods described below in conjunction with blocks 260-262). The Vsense signals received from the selected sensing channel 83 or 85 is used for adjusting the VTI/VFI counters for VT/VF detection and may be used for bradycardia sensing methods until control circuit 80 transitions back to the unconcerned sensing state 1 from either of the tachyarrhythmia operating states 2 or 5 (see FIG. 5).

Referring again to block 256, when the NID is reached for only one sensing channel 83 or 85, e.g., when a VTI/VFI counter associated with one sensing channel 83 or 85 has not reached an NID but a VTI/VFI counter associated with the other sensing channel has reached an NID, control circuit 80 may advance to block 260. At block 260, control circuit 80 may determine sensed event metrics from the sensed event data buffered in memory 252 for use in selecting a reliable sensing channel. Control circuit 80 may analyze the buffered sensed event data for determining which sensing channel 83 or 85 is deemed most reliable for detecting VT/VF.

At block 260, control circuit 80 may determine sensed event metrics for each sensing channel 83 and 85 from the sensed event data buffered at block 252. The sensed event data may be retrieved for a specified number of most recent Vsense signals for each sensing channel 83 and 85. The sensed event data may be retrieved for all Vsense signals received from both sensing channels 83 and 85 during a specified time interval preceding the NID being reached by one sensing channel 83 or 85. In some examples, the sensed event metrics may be determined from sensed event data buffered for Vsense event signals received over a most recent, specified time interval, e.g., over the most recent 3 seconds, up to a specified maximum number of most recent Vsense events. For the sake of illustration, sensed event metrics can be determined for the most recent M Vsense signals from each sensing channel, where M may be the same or different for each sensing channel and the M Vsense signals occur within the most recent 3 seconds or less prior to the NID being reached for one of the sensing channels 83 or 85.

The sensed event metrics are determined for each sensing channel 83 and 85 from the sensed event data store for the respective sensing channel. The sensed event metrics determined by control circuit 80 at block 260 may include a maximum RRI out of the buffered RRIs determined for each sensing channel 83 and 85. The sensed event metrics determined at block 260 by control circuit 80 may include a representative peak amplitude, e.g., a mean peak amplitude, determined from the peak amplitudes buffered for Vsense signals for each sensing channel 83 and 85.

The sensed event metrics determined for each sensing channel 83 and 85 by control circuit 80 at block 260 may include a matched events ratio. The matched events ratio can be determined as the ratio of the number of Vsense signals classified as matched events to the total number M of Vsense signals being evaluated for the respective sensing channel 83 or 85.

The sensed event metrics determined for each sensing channel 83 and 85 by control circuit 80 at block 260 may include a matched to unmatched event amplitude ratio (MUAR). The MUAR can be determined as the median (or other representative value) of the peak amplitudes determined for Vsense signals classified as matched event signals and the median (or other representative value) of the peak amplitudes determined for all Vsense signals classified as unmatched event signals. In some examples, if all Vsense signals of the M Vsense signals for a given sensing channel 83 or 85 are classified as matched events, the MUAR may be set to 0.

The sensed event metrics determined for each sensing channel 83 and 85 by control circuit 80 may include a representative ASTR, e.g., a mean ASTR, determined from the ASTRs buffered for the Vsense signals for each respective sensing channel. The sensed event metrics determined for each sensing channel 83 and 85 by control circuit 80 may include a noisy beat count. The noisy beat count may be the number of Vsense signals classified as a noisy event based on the noise metric, e.g., the signal pulse count during the in-channel blanking period, being greater than a noise threshold, e.g., more than 3, 5, 7 or other selected noisy event threshold.

At block 262, control circuit 80 identifies the sensing channel 83 or 85 associated with the NID being reached as the “candidate” sensing channel. The other sensing channel 85 or 83, associated with VTI/VFI counters that have not reached an NID, may be referred to as the “non-candidate” sensing channel for the sake of convenience. Control circuit 80 may determine if the sensed event metrics determined at block 260 meet candidate sensing channel reliability criteria at block 262. In some examples, multiple combinations of criteria may be applied to the sensed event metrics to determine which one of the candidate sensing channel or the non-candidate sensing channel is deemed most reliable for sensing ventricular event signals for VT/VF detection.

In various examples, one combination of criteria that verifies reliable sensing by the candidate sensing channel during likely VT/VF may be applied to the sensed event metrics by control circuit 80. The likely VT/VF criteria may include a criterion applied to the matched events ratio for the candidate sensing channel. The likely VT/VF criterion may include a criterion applied to the maximum RRI determined for the non-candidate sensing channel. For example, when the matched events ratio for the candidate sensing channel is greater than or equal to a threshold ratio and the maximum RRI for the non-candidate sensing channel is less than a threshold interval, control circuit 80 may determine that the likely VT/VF criteria are met. The matched events ratio for the candidate sensing channel may be required to be at least 0.7, 0.8 or 0.9, as examples. The maximum RRI for the non-candidate sensing channel may be required to be less than the longest VT or VF detection interval in effect plus an offset, for example. The offset may be 30 ms, 40 ms, 50 ms or 60 ms as examples. The candidate sensing channel reliability criteria may be met at block 262 based on the likely VT/VF criteria being met by the matched events ratio of the candidate sensing channel and the maximum RRI of the non-candidate sensing channel. The likely VT/VF criteria can be satisfied by the sensed event metrics when the NID is reached for one sensing channel, but not both, due to some jittering in the timing of Vsense signals received from the non-candidate sensing channel. Selecting the candidate sensing channel as the reliable sensing channel and transitioning to the concerned state 2 (block 266) avoids delaying or missing a VT/VF detection when jitter in the RRIs determined from one sensing channel delays the VTI/VFI counters associated with the non-candidate sensing channel from reaching the NID at the same time as the candidate sensing channel.

Additionally or alternatively, control circuit 80 may apply a second combination of criteria to the sensed event metrics for verifying reliable sensing and unlikely oversensing by the candidate sensing channel. In one example, this combination of criteria may include applying a criterion to the noisy beat count determined for the candidate sensing channel. This combination of criteria may include applying a threshold to the MUAR determined for the candidate sensing channel. This combination of criteria may include applying a threshold to the mean ASTR of the candidate sensing channel. In some examples, this combination of criteria may include applying a threshold to a ratio of the mean ASTR determined for the candidate sensing channel to the mean ASTR determined for the non-candidate sensing channel. This ratio of the mean ASTRs can be referred to as the candidate ASTR to non-candidate ASTR ratio. In an example, when the noisy beat count is zero and the MUAR is less than or equal to 1.3 for the candidate sensing channel and the ratio of the candidate ASTR to non-candidate ASTR is greater than or equal to 0.9, control circuit 80 may determine that the candidate sensing channel reliability criteria are met at block 262 based on likely reliable sensing and unlikely oversensing by the candidate sensing channel. The noisy beat count threshold may be 0, 1 or 2 in various examples, The MUAR threshold may be 1.2, 1.3, 1.4 or 1.5 in various examples. The threshold applied to the ratio of the candidate ASTR to non-candidate ASTR may be 0.8, 0.9 or 1.0 in various examples. These and other example values of various thresholds, ranges or other criteria applied to the sensed event metrics are intended to be illustrative and non-limiting in nature. Selecting the candidate sensing channel and transitioning to the concerned state 2 (block 266) when the sensed event metrics indicate reliable sensing without oversensing by the candidate sensing channel enables ICD 14 to detect a VT/VF episode with minimal delay when the non-candidate sensing channel has not reached an NID at the same time as the candidate sensing channel.

Additionally or alternatively, control circuit 80 may apply a third combination of criteria to the sensed event metrics to verify likely undersensing by the non-candidate sensing channel at block 262, thereby indicating that the candidate sensing channel is the more reliable sensing channel for VT/VF detection. In one example, this combination of criteria may include applying a threshold to the mean peak amplitude determined for the non-candidate sensing channel. This combination of criteria may include applying a threshold interval to the maximum RRI determined for the non-candidate sensing channel. For instance, when the mean peak amplitude determined for the M Vsense signals buffered for the non-candidate channel is less than a specified multiple of the sensitivity for that sensing channel and the maximum RRI is greater than the threshold interval, the non-candidate sensing channel may be undersensing low amplitude R-waves or fibrillation waves. The candidate sensing channel reliability criteria may be met at block 262 due to likely undersensing by the non-candidate sensing channel. The specified multiple of the sensitivity may be 1.5, 2.0 2.5, 2.75, 3.0, 3.25 or 3.5 times the sensitivity currently in effect for that sensing channel, which may be the programmed sensitivity or an increased sensitivity due to classification of a cardiac signal segment as VT/VF based on GMA (e.g., as described above in conjunction with FIG. 6). The threshold interval may be 1250 ms, 1500 ms, 1750 ms, 2000 ms or other specified threshold interval. When the mean peak amplitude is relatively small (less than a multiple of the sensitivity) and at least one relatively long RRI has occurred in the last M Vsense events, undersensing on the non-candidate sensing channel may be likely. Selecting the candidate sensing channel and transitioning to the concerned state 2 (block 266) may minimize the likelihood of a delayed detection of a VT/VF episode due to undersensing by one sensing channel 83 or 85.

Control circuit 80 may transition to the concerned state 2 at block 266 in response to any of the applied candidate sensing channel reliability criteria being met at block 262. The candidate sensing channel can be the selected sensing channel that is used by control circuit 80 for determining RRIs and other analyses performed for detecting VT/VF. Vsense event signals received from only the candidate sensing channel may be used by control circuit 80 for detecting a long pause and resetting bradycardia pacing escape intervals and/or other time intervals used for detecting a long pause and for controlling delivery of bradycardia pacing pulses by therapy delivery circuit 84. In some examples, the non-candidate sensing channel may be powered down during the concerned state 2 for conserving power source 98.

When control circuit 80 determines that the candidate sensing channel reliability criteria are not met, control circuit 80 may remain in the unconcerned sensing state 1 (at block 270). In this case, the non-candidate sensing channel may be deemed the reliable sensing channel such that the NID being reached by the candidate sensing channel may be falsely reached, e.g., due to oversensing. Because the non-candidate sensing channel is the channel having VTI/VFI counters that did not reach the NID, control circuit 80 does not transition to the concerned sensing state 2. The process of flow chart 250 may return to block 251. Control circuit 80 may continue to receive Vsense signals from both sensing channels 83 and 85 and determine sensed event data at block 252 until either (or both) sensing channels 83 and/or 85 reach an NID (or a threshold number of cardiac signal segment are classified as VT/VF and a specified percentage of the NID is reached).

While examples of three different combinations of criteria are described here for determining when candidate sensing channel reliability criteria are met, it is to be understood that fewer than or more than three combinations of criteria may be applied to the sensed event metrics determined for one or both sensing channels in various examples. Furthermore, different sensed event data and/or different sensed event metrics than those described here may be determined for use in ascertaining when one sensing channel 83 or 85 is more reliable than the other sensing channel 85 or 83 due to likely undersensing, oversensing or reliable sensing without undersensing or oversensing by a given sensing channel when one sensing channel reaches the NID and the other does not. Methods performed by sensing circuit 86 and control circuit 80 for selecting a reliable sensing channel for VT/VF detection and controlling when a transition from the unconcerned state 1 to the concerned state 2 occurs may include aspects of the techniques generally disclosed in U.S. patent application Ser. No. 17/823,055 (Liu, et al.), filed on Aug. 29, 2022, the content of which is incorporated herein by reference in its entirety.

FIG. 8 is a flow chart 300 of a method that may be performed by control circuit 80 for detecting suspected VT/VF undersensing during the unconcerned sensing state 1 of FIG. 6. As described above in conjunction with FIG. 7, control circuit 80 may determine and store sensed event data for each Vsense signal received from each sensing channel 83 and 85 during the unconcerned sensing state 1. Sensed event data may be stored in a first-in-first-out buffer for example, for the most recent 8, 10, 16, 20, 30 or other specified number of Vsense signals for each sensing channel. Control circuit 80 may analyze the sensed event data for determining when suspected VT/VF undersensing criteria are met.

In various examples, control circuit 80 may analyze the RRIs, peak amplitudes, and/or morphology, of sensed event signals for determining if VT/VF undersensing may be occurring. In general, VT/VF undersensing is occurring when R-waves and/or fibrillation waves are undersensed by sensing circuit 86 such that VTIs and/or VFIs are undercounted at a given point in time, which may delay or prevent an NID from being reached. The process of flow chart 300 includes examples of various criteria, e.g., thresholds or other values, that may be applied to sensed event data or metrics of sensed event data for determining that undersensing of R-waves and/or fibrillation waves during a possible VT/VF episode is likely to be occurring.

At block 302, control circuit 80 may determine a maximum RRI from among the RRIs buffered for both sensing channels 83 and 85. When an RRI determined for a Vsense signal received from either sensing channel 83 or 85 is greater than or equal to an undersensing threshold interval as determined at block 304, control circuit 80 may determine that suspected undersensing criteria are met at block 306. The undersensing threshold interval may be, for example, 1.5 seconds to 4 seconds and is 2.5 seconds in one example.

As described above in conjunction with FIG. 6, control circuit 80 may initiate buffering and GMA of a cardiac signal segment in response to the suspected VT/VF undersensing detection. In some examples, if buffering and GMA of a cardiac signal segment is already underway, e.g., due to a suspected long pause detection according to bradycardia sensing methods, the result of that GMA may be used by control circuit 80 for responding to the suspected VT/VF undersensing detected at block 306, rather than buffering a new cardiac signal segment.

In some examples, if the maximum RRI is not at least the undersensing threshold interval (“no” branch of block 304), amplitude and/or morphology criteria may be applied to sensed event data when a VTI/VFI counter has started counting up but has not yet reached an NID. At block 308, control circuit 80 may determine if a VTI/VFI count associated with either sensing channel 83 or 85 is greater than a low threshold value that is less than the NID. The low threshold value may be 2, 3, 4, 5, 8, 10 or other selected threshold. The low threshold value is referred to as being “low” because it can be a lower value or percentage of the NID than the threshold VTI/VFI counter required to transition to tachyarrhythmia operating state 2 when a threshold number of cardiac signal segments are classified as VT/VF based on the GMA (e.g., as shown by arrow 130 of FIG. 6 or described in conjunction with block 255 of FIG. 7). If the VTI/VFI counts are all zero or less than the low threshold value for both sensing channels 83 and 85 (“no” branch of block 308), control circuit 80 may determine that suspected VT/VF undersensing criteria are not met at block 322.

If a VTI/VFI counter associated with one sensing channel 83 or 85 is greater than the low threshold value (“yes” branch of block 308) but not both sensing channels 83 and 85 (“no” branch of block 310), and an NID is not reached for either sensing channel, control circuit 80 may advance to block 312 to determine and analyze amplitude metrics determined from buffered sensed event data stored for each sensing channel 83 and 85. For example, at block 312, control circuit 80 may determine an amplitude metric based on the peak amplitudes buffered for the Vsense signals for each sensing channel 83 and 85. As described above in conjunction with FIG. 7, control circuit 80 (or sensing circuit 86) may determine the maximum rectified peak amplitude during the in-channel blanking period for each Vsense signal received from sensing channels 83 and 85. At block 312, control circuit 80 may determine an amplitude metric for each sensing channel 83 and 85 as a representative value of the peak amplitudes, e.g., a mean, median, minimum, maximum or other representative value, buffered for each respective sensing channel 83 and 85. In one example, the median peak amplitude is determined for each sensing channel 83 and 85 from twelve (or other selected number of) buffered peak amplitudes stored for the respective sensing channel 83 or 85.

At block 314, control circuit 80 may compare the amplitude metrics, e.g., the representative peak amplitude determined for each sensing channel 83 and 85, to an undersensing threshold amplitude. When the representative peak amplitudes determined for sensing channel 83 and for sensing channel 85 are each less than a respective undersensing threshold amplitude, control circuit 80 may advance to block 316 for determining R-wave morphology matching scores for the next N Vsense signals. In other examples, control circuit 80 may detect suspected VT/VF undersensing at block 306 in response to the amplitude metrics being less than the respective undersensing thresholds at block 314 without requiring a determination and analysis of R-wave morphology matching scores.

Control circuit 80 may apply the undersensing threshold amplitude as a multiple of the programmed sensitivity for each respective sensing channel 83 and 85. When the process of flow chart 300 is performed at block 120 of FIG. 6), the programmed sensitivity for each sensing channel 83 and 85 can be in effect. The undersensing threshold amplitudes applied to the representative peak amplitudes determined from each sensing channel 83 and 85 may be different from each other or the same. Each undersensing threshold amplitude may be set based on the respective sensitivity of the sensing channel 83 or 85 using the same multiple or a different multiple of the sensitivity, for example. The multiple of the sensitivity used to set the undersensing threshold amplitude may range from 2 to 6 and may be between 2.5 and 5 as examples.

In an illustrative example, when the median peak amplitude determined from buffered sensed event peak amplitudes for a first sensing channel 83 or 85 is less than 2.6 times the programmed sensitivity of the first sensing channel and the median peak amplitude determined from buffered sensed event peak amplitudes for the second sensing channel 85 or 83 is less than 5 times the programmed sensitivity for the second sensing channel, control circuit 80 may determine that the amplitude metrics are less than respective undersensing thresholds at block 314. The first sensing channel may be the sensing channel having a VTI/VFI count greater than the threshold (as determined at block 310), and the second sensing channel may be the sensing channel having a VTI/VFI count less than the threshold (as determined at block 310).

If the amplitude metric(s) are not less than the respective undersensing threshold(s) (“no” branch of block 314), control circuit 80 may determine that suspected VT/VF undersensing criteria are not met at block 322. Suspected VT/VF undersensing is not detected. Sensing circuit 86 may continue to operate according to the programmed sensitivity, e.g., at block 120 of FIG. 6. No sensitivity adjustments may be made by sensing circuit 86, and GMA may remain disabled (unless already enabled due to a suspected long pause detection in which case control circuit 80 advances to block 122 of FIG. 6).

When the amplitude metric(s) are less than the respective undersensing threshold(s) at block 314, control circuit 80 may detect suspected VT/VF undersensing at block 306 in some examples. However, in the example shown, control circuit 80 may be configured to determine R-wave morphology matching scores and/or RRIs for the next N Vsense signals for determining if suspected VT/VF undersensing criteria are met. As such, control circuit 80 may advance to block 316 in response to the amplitude metrics being less than the respective undersensing thresholds (“yes” branch of block 314).

Referring again to block 310, if VTI/VFI counters associated with both sensing channels 83 and 85 are greater than the low threshold value, e.g., a VFI count for sensing channel 83 is at least 5 and a VFI count for sensing channel 85 is at least 5 (“yes” branch of block 310), control circuit 80 may omit the amplitude analysis performed at blocks 312 and 314 in some examples. Control circuit 80 may proceed directly to block 316 for determining R-wave morphology matching scores for the next N Vsense signals.

In some examples, when a VTI/VFI counter reaches the low threshold value, e.g., 2, 3, 4 or 5, control circuit 80 may enable buffering of sensed event signal segments of the morphology signal received from morphology sensing channel 87. Morphology signal channel 87 may be powered down or disabled to conserve power source 98 until the cardiac electrical signal that is sensed by morphology signal channel 87 (also referred to herein as the “morphology signal”) is needed by control circuit 80 for R-wave morphology matching, GMA or other morphology related analyses. Sensed event morphology analysis of a sensed event signal segment obtained in time relation to a Vsense signal may be enabled by control circuit 80 when a VTI/VFI counter reaches the low threshold value or another specified value. The sensed event morphology analysis may be used for determining when suspected VT/VF undersensing criteria are met in some examples.

For example, starting when a VTI/VFI counter reaches the low threshold value, control circuit 80 may determine an R-wave morphology matching score for each Vsense signal. The R-wave morphology matching score may be buffered with other sensed event data determined for each Vsense signal received after a VTI/VFI counter reached the low threshold value. In other examples R-wave morphology matching scores may be determined for each Vsense signal, even before any of the VTI/VFI counters reach the low threshold value. To illustrate, control circuit 80 may begin determining and storing R-wave morphology matching scores for each Vsense signal when a VTI/VFI counter reaches 3. The low threshold value applied at blocks 308 and 310 may be 5 so that when the low threshold value is reached by at least one sensing channel, R-wave morphology matching scores for one or more of the most recent preceding Vsense signals can already be available for analysis at block 318.

At block 316 control circuit 80 may determine the R-wave morphology matching score, also referred to herein as a “matching score,” for each of N Vsense signals received from one or both sensing channels. The N Vsense signals may be the next N Vsense signals received after the low threshold is reached by the VTI/VFI counters of at least one sensing channel 83 or 85. The value of N may be 3, 4, 6, 8, 10 or other specified number. In some examples, the N Vsense signals may include one or more Vsense signals occurring before the low threshold is reached when morphology matching scores are available for one or more most recent preceding Vsense signals. An R-wave template may be previously stored in memory 82 for use in determining matching scores. The R-wave template may be established by control circuit 80 from the morphology signal received from morphology sensing channel 87 based on sensed event signal segments that are known or likely normal sinus R-waves. A variety of methods may be used for establishing an R-wave template that can be stored in memory 82 for determining matching scores.

Control circuit 80 may determine matching scores at block 316 by buffering a cardiac signal segment received from morphology sensing channel 87. When a Vsense signal is received from either sensing channel 83 or 85, a sensed event signal segment that may extend before and/or after the timing of the Vsense signal may be retrieved by control circuit 80 from the morphology signal for determining a matching score. The sensed event signal segment may extend for 48 samples of a 256 Hz sampled morphology signal segment that is centered on the time of the Vsense signal in an example. Control circuit 80 may perform a R-wave morphology matching analysis to obtain matching score between the sensed event signal segment and the stored R-wave template. Morphology matching techniques may include performing a wavelet transform, waveform correlation or other methods. The matching score may range from 0 to 100 in an example. A variety of morphology matching techniques may be used for determining a matching score for a sensed event signal segment based on a comparison to a previously established R-wave template (e.g., stored in memory 82).

At block 318, control circuit 80 may determine a count of the matching scores that are less than a match threshold out of the N morphology matching scores determined for the N Vsense signals received from either (or both) sensing channels 83 and 85. The match threshold may be 40, 50, 60, or 70 as examples. A matching score greater than or equal to the match threshold is indicative of a sensed waveform that matches a normal sinus R-wave with a high degree of confidence. If less than M morphology matching scores determined for the N Vsense signals received from a given sensing channel 83 or 85 are less than the match threshold (“no” branch of block 318), control circuit 80 may determine that suspected VT/VF undersensing criteria are not met at block 322. When fewer than M matching scores are less than the match threshold, meaning that more than N-M matching scores are greater than the match threshold, the Vsense signals associated with the matching scores greater than the match threshold may correspond to normal sinus R-waves and be a contraindication for VT/VF. Control circuit 80 may remain in block 120 of the unconcerned sensing state 1 (see FIG. 6) without adjusting the sensitivity of sensing channels 83 or 85.

Referring again to block 318, when at least M out of the N matching scores are less than the match threshold, control circuit 80 may advance to block 320 to evaluate the N RRIs determined for the N Vsense signals for one or both sensing channels 83 and/or 85. In some examples, control circuit 80 determines a matching score for each of the next 6 Vsense signals received from at least one sensing channel 83 or 85 that reached a VTI/VFI count greater than or equal to the low threshold value. When at least 5 of the 6 matching scores are less than the match threshold, for example, control circuit 80 may advance to block 320 to analyze the RRIs for the 6 Vsense signals. In other examples, at least 70%, 80%, or 90% of the matching scores may be required to be less than the match threshold at block 318.

At block 320, control circuit 80 may verify that fewer than a threshold number of the RRIs determined for the N Vsense signals (for which morphology matching scores were determined) are VTIs or VFIs. In an example, less than 4 of the RRIs determined for 6 Vsense signals received from a given sensing channel 83 or 85 may be counted as a VTI or VFI in order for control circuit 80 to detect suspected VT/VF undersensing at block 306. If more than X, e.g., 4, of the N RRIs are counted as VT/VF intervals (“no” branch of block 320), the VTI counter and/or VFI counter may be trending upward toward the NID. In this case, GMA need not be triggered or the sensitivity increased to avoid undersensing of VT/VF because the NID may be reached by the VTI/VFI counters when sensing circuit 86 is operating according to the programmed sensitivity. As such, if at least X out of N RRIs (e.g., at least 50%, 60%, 70% or 80% of the N RRIs), are counted as VT/VF intervals (“no” branch of block 320), control circuit 80 may determine that suspected VT/VF criteria are not met at block 322.

In general, suspected VT/VF undersensing may be detected when the peak amplitudes buffered for Vsense signals are relatively small (less than a multiple of the sensitivity in effect), R-wave morphology matching scores are relatively low (at least M morphology matching scores less than the match threshold), and few (if any) RRIs are being counted as VT/VF intervals (less than X of N VT/VF intervals such that VT/VF intervals may be undercounted due to undersensing of low amplitude R-waves or fibrillation waves). The comparisons at block 314, 318 and 320 check for each of these conditions for detecting suspected VT/VF undersensing by control circuit 80 according to some examples.

In the example shown in FIG. 8, control circuit 80 determines that suspected VT/VF undersensing criteria are met at block 306 when at least one RRI is greater than or equal to the undersensing threshold interval (decision block 304) or when criteria applied to the VTI/VFI counters, sensed event amplitude metrics, morphology matching scores and/or RRIs (e.g., any combination of decision blocks 308-320) are met. In other examples, one or more requirements relating to RRIs, sensed event peak amplitudes, and/or morphology matching scores or any combination thereof may be applied as criteria for determining when suspected VT/VF undersensing criteria are met. The criteria shown in the decision blocks of FIG. 8 may be applied in a different order or combination than shown in flow chart 300 and some decision blocks may be omitted in some examples. For instance, when a VTI count or a VFI count is greater than a specified threshold and less than the NID for either or both sensing channels 83 and 85, control circuit 80 may perform the amplitude analysis at blocks 312 and 314 with or without performing the R-wave morphology matching analysis at blocks 316 and 318. In other examples, the morphology analysis may be performed at blocks 316 and 318 with or without performing the amplitude metric analysis at blocks 312 and 314. Any combination of amplitude criteria, RRI criteria and/or R-wave morphology matching criteria may be applied by control circuit 80 to buffered sensed event data and/or in association with subsequent Vsense event signals for detecting suspected VT/VF undersensing. Other examples of methods that may be used for detecting suspected VT/VF undersensing are generally disclosed in provisional U.S. Patent Application 63/310,558 (Heinks, et al.), filed on Feb. 15, 2022, the content of which is incorporated herein by reference in its entirety.

It is to be understood that in some examples, the process of flow chart 300 is performed for detecting suspected VF undersensing when VT detection is disabled. However, even if VT detection is enabled, the process of flow chart 300 may be performed for primarily detecting suspected VF undersensing such that at blocks 308 and 310, control circuit 80 may determine if the VFI counter for each sensing channel 83 and 85 is at least a threshold value (and less than the NID for detecting VF). At block 320, the RRIs determined for the N Vsense signals may be compared to the VF detection interval for determining when less than X VFIs are counted from the RRIs determined for the N Vsense signals.

When suspected VT/VF undersensing criteria are met (as determined at block 306), control circuit 80 may trigger the GMA of a cardiac signal segment as described above in conjunction with FIG. 6 (e.g., at block 124 of FIG. 6). In some instances, a GMA may already be underway due to a suspected long pause detection according to bradycardia sensing methods. In this case, the result of the bradycardia-triggered GMA may be used for responding to the detection of the suspected VT/VF undersensing without necessarily buffering a new cardiac signal segment. When the cardiac signal segment is classified as a VT/VF segment based on the GMA, control circuit 80 may increase the sensitivity of one or both sensing channels 83 and 85 as shown by block 122 of FIG. 6 and continue buffering at least one more cardiac signal segments for performing the GMA. When the next cardiac signal segment is classified as non-VT/VF or asystole, control circuit 80 may return to block 120 of FIG. 6 and may restore the sensitivity of sensing channels 83 and 85 to the programmed sensitivity. Control circuit 80 may disable buffering of cardiac signal segments for the GMA.

FIG. 9 is a flow chart 350 of a method for performing GMA by control circuit 80 according to some examples. The GMA may be performed in response to detecting a suspected long pause according to bradycardia sensing methods or in response to detecting suspected VT/VF undersensing according to tachyarrhythmia sensing methods. As shown in FIG. 6, control circuit 80 may trigger the GMA of a cardiac signal segment to be performed in response to detecting suspected VT/VF undersensing, e.g., using the techniques described in conjunction with FIG. 8. As described above, control circuit 80 may trigger the GMA of a cardiac signal segment to be performed in response to detecting suspected long pause. Control circuit 80 may trigger the GMA of a cardiac signal segment upon starting a long pause confirmation interval, e.g., upon transitioning to redetection state 5 as described below in conjunction with FIG. 13.

Once GMA is enabled during the unconcerned state 1 in response to a triggering event such as a suspected long pause detection or a suspected VT/VF undersensing detection, the GMA of flow chart 350 may be repeated for at least one additional cardiac signal segment if the first cardiac signal segment is classified as VT/VF. When control circuit 80 transitions to any of tachyarrhythmia operating states 2, 3 or 5, the GMA of flow chart 350 may remain enabled. As such, the process of flow chart 350 may be performed any time control circuit 80 needs the result of the GMA for use in bradycardia sensing and pacing control and/or for use in detecting VT/VF and for controlling CV/DF shock therapy.

At block 351, control circuit 80 obtains a cardiac signal segment for performing the GMA. The cardiac signal segment may be received from the morphology signal channel 87. The cardiac signal segment may be at least one second and can be 1.5 to 6 seconds long in various examples. In an illustrative example, the cardiac signal segment is 3 seconds long and its starting point is independent of the timing of a Vsense signal because there may not be any Vsense signals being received, e.g., if a long pause in ventricular activity or VT/VF undersensing is occurring.

The GMA of a cardiac signal segment is different than the R-wave morphology matching or other morphology analysis of a sensed event signal segment that is associated with a Vsense signal due to an R-wave threshold crossing. The R-wave morphology matching or other morphology analysis of a sensed event signal segment is performed on a sensed event signal segment that is acquired having beginning and end times that are in timed relation to a Vsense signal and is intended to encompass one R-wave or QRS waveform, for example. The cardiac signal segment obtained at block 351 for GMA may be relatively long compared to a cardiac cycle length, e.g., greater than a VT or VF detection interval, and may therefore include multiple VT or VF cycles when VT/VF is present. As such, the GMA may be performed on cardiac signal segments having beginning and ending times that do not necessarily encompass the time of a Vsense signal (because no Vsense signals may be being received). The GMA may be performed to assess the frequency content of the cardiac signal segment for an indication of relatively high frequency VT/VF cycles that may be present in the cardiac signal segment without knowing the relative timing of the VT/VF cycles or R-waves or fibrillation waves.

At block 352, control circuit 80 may determine an amplitude metric from the cardiac signal segment. The amplitude metric may be determined by identifying signal peaks in the cardiac signal segment and determining a representative amplitude of the identified signal peaks. In some examples, control circuit 80 may determine a first derivative signal from the cardiac signal segment, which may be estimated as a first order difference signal by determining the difference between consecutive sample points of the cardiac signal segment. The derivative signal can be rectified for facilitating analysis of the cardiac signal segment for identifying signal peaks. In some examples, a gradient signal is determined from the morphology signal received from morphology signal channel 87, which may be subsequently bandpass filtered, prior to determining the rectified, difference signal from the cardiac signal segment.

Control circuit 80 may identify signal peaks of the rectified difference signal that are greater than a signal pulse amplitude threshold and separated by at least a threshold time interval from other signal peaks. For example, starting from the beginning of the cardiac signal segment, the first earliest signal pulse that is greater than the signal pulse amplitude threshold may be identified. The next earliest signal pulse that is greater than the signal pulse amplitude threshold and has a peak that is at least 100 ms later than the first signal pulse peak may be identified and so on. In this way, the next earliest signal pulse that is at least 100 ms after the most recently identified signal pulse is identified as a signal pulse. In other examples, if more than one signal pulse peak that is greater than the signal pulse amplitude threshold occurs within 100 ms of each other, the signal pulse peak having the greatest amplitude may be identified as a signal pulse peak and other signal pulse peaks that are within 100 ms of the identified signal pulse peak may be ignored. In this way, a single R-wave or fibrillation wave that may have more than one peak is not identified twice.

Control circuit 80 may determine the signal pulse amplitude threshold as a percentage, e.g., 15 to 50% or about one-sixth to one-half, of an average of local maximum amplitudes of multiple subsegments of the cardiac signal segment. In an illustrative example, control circuit 80 may determine the local maximum peak amplitude of each of four subsegments of the 3-second cardiac signal segment. Control circuit 80 may determine the signal pulse amplitude threshold as one-third of the mean amplitude of the four local maximum amplitudes.

Control circuit 80 may determine an amplitude metric from the identified signal pulses at block 352. Control circuit 80 may determine the amplitude metric by determining a mean, median, maximum, minimum, range, and/or standard deviation or other representative value(s) of the peak amplitudes of the signal pulses identified as being greater than the signal pulse amplitude threshold and at least the minimum time interval apart. At block 354, control circuit 80 may compare the amplitude metric to an amplitude threshold. The amplitude threshold may be between 0.05 and 0.2 millivolts, as examples. In one example the amplitude threshold is 0.1 millivolt but other thresholds may be applied to the amplitude metric. The amplitude threshold may be based on the minimum expected amplitude of fibrillation waves. The amplitude threshold may be referred to as an “asystole amplitude threshold” because the amplitude threshold may represent a minimum amplitude of the cardiac signal segment when ventricular activity is present, e.g., R-waves or fibrillation waves. When the amplitude metric is less than the amplitude threshold, ventricular asystole may be present. When the amplitude metric is less than the amplitude threshold (“yes” branch of block 354), control circuit 80 may perform a first analysis of the cardiac signal segment for determining if asystole criteria are met at block 356. When the amplitude metric is greater than (or equal to) the amplitude threshold (“no” branch of block 354), control circuit 80 may perform a second analysis of the cardiac signal segment for determining if VT/VF criteria are met at block 360.

The asystole analysis performed at block 356 may generally include determining that the cardiac signal segment amplitude variation is consistently within an asystole range for at least a specified portion of the cardiac signal segment. During asystole, the cardiac signal segment is expected to be relatively flat with a low degree of signal fluctuations. The asystole analysis of the cardiac signal segment may include determining a signal stability metric that is representative of the degree of signal fluctuations present in the cardiac signal segment. In some examples, a signal stability metric can be determined as a count of a number of moving windows in the cardiac signal segment during which less than a threshold number of sample points of the signal segment fall outside, e.g., are greater than or less than, an asystole amplitude range.

At block 356, control circuit 80 may determine a low pass filtered signal to smooth the cardiac signal segment. Signal smoothing by averaging or filtering may result in a relatively flat signal when no ventricular activity is present and enhance any ventricular activity signals relative to attenuated noise signals that may be present in the cardiac signal segment. Control circuit 80 may be configured to determine a low pass filtered signal from the wideband and notch filtered cardiac signal received from morphology signal channel 87. In some examples, a bandpass filter may be applied to the wideband and notch filtered cardiac signal received from morphology signal channel 87 and used for determining when asystole criteria are met. Control circuit 80 may determine a gradient signal from the low pass filtered signal. The gradient signal may be generated by a central difference method in some examples, e.g., by determining the difference between the i+1 and i−1 sample points and dividing by 2. In this way, a signal representing the gradient or rate of change of the low pass filtered cardiac signal segment is determined. In other examples, a forward difference or backward difference method could be used.

Control circuit 80 may determine if the cardiac signal segment meets asystole criteria when the cardiac signal sample points (e.g., of the gradient signal) remain within an asystole range for at least a specified number of sample points or specified cumulative time interval, which may be all or a portion of the cardiac signal segment. When asystole is present, the gradient signal is expected to be a substantially flat signal with small fluctuations. If asystole is not present, ventricular activity will cause fluctuations in the gradient signal that exceed the asystole range. When the gradient signal exceeds the asystole range for a threshold number of sample points of the gradient signal, control circuit 80 may determine that the cardiac signal segment is not an asystole segment. When the gradient signal is within the asystole range for at least a threshold number of sample points out a specified number of consecutive or non-consecutive sample points (or predetermined time interval), control circuit 80 may determine that a persistent asystole condition exists and classify the cardiac signal segment as being an asystole segment.

In some examples, control circuit 80 may determine that asystole criteria are met at block 356 when at least Y consecutive running windows of Z sample points of the cardiac signal segment include at least X sample points of the gradient signal that fall within the asystole range. In an illustrative example, when at least 5 sample points out of a running window of 21 sample points of the gradient signal are within the asystole range for 100 to 700 consecutive running windows, or other portion of the cardiac signal segment or number of sample points, control circuit 80 may determine that the asystole criteria are met at block 356. The cardiac signal segment can be classified as an asystole segment by control circuit 80 at block 358 in response to the asystole criteria being met at block 356. When the asystole criteria are not met at block 356, control circuit 80 may classify the cardiac signal segment as non-VT/VF at block 362.

Referring again to block 354, when control circuit 80 determines that the amplitude metric is not less than the amplitude threshold, the signal pulses identified at block 352 for determining the amplitude metric may correspond to ventricular activity, e.g., R-waves or fibrillation waves. At block 360, control circuit 80 may determine if VT/VF criteria are met by the cardiac signal segment. Control circuit 80 may determine one or more morphology metrics from the cardiac signal segment for detecting evidence of VT/VF. The morphology metric(s) determined from the cardiac signal segment may discriminate between true VT/VF rhythms and non-VT/VF rhythms, which may include supraventricular tachycardia, rapidly conducted atrial fibrillation, or oversensing of P-waves, T-waves, and/or non-cardiac noise. The morphology metrics can be determined from sample points spanning the entire cardiac signal segment and are not dependent on identifying cardiac signal segments that include the time of a Vsense signal received from sensing channel 83 or 85. In some instances, no Vsense signals may be received from sensing circuit 86 during the cardiac signal segment being analyzed for VT/VF detection.

As described above, the cardiac signal segment may be received from the morphology signal channel 87 and may be a bandpass and notch filtered signal. In some examples, the cardiac electrical signal is filtered, e.g., using a 2 Hz to 40 Hz bandpass filter, a 3 Hz to 32 Hz bandpass filter, or a 4 Hz to 30 Hz bandpass filter. Control circuit 80 may determine a gradient signal and/or bandpass filtered signal from the signal received from morphology signal channel 87 followed by determining a first order difference signal that is rectified for determining the VT/VF morphology metric(s) from the cardiac signal segment.

One morphology metric relating to the frequency content of the cardiac signal segment that may be determined by control circuit 80 is a mean period (MP). The MP may be calculated as the inverse of the mean frequency of the cardiac signal segment, which is an estimate of the center frequency of the cardiac signal segment. The mean frequency may be determined by control circuit 80 as the ratio of the average absolute amplitude of the rectified first order difference signal (sum of all sample point amplitudes of the rectified difference signal divided by the total number of sample points) to the average absolute amplitude of the rectified cardiac signal segment (sum of all sample point amplitudes of the rectified cardiac signal segment divided by total number of sample points). As such, the MP may be estimated as the ratio of the sum of all sample point amplitudes of the rectified cardiac signal segment to the sum of all sample point amplitudes of the rectified first order difference signal. The MP may optionally be converted to a radian measure by multiplying this ratio by the factor 27π/(sampling frequency).

In some examples, control circuit 80 may use the MP for determining the spectral width (SW) at block 360. Control circuit 80 may determine the SW as the fundamental period of the cardiac signal segment less the MP. In some examples, control circuit 80 may determine the fundamental period as the mean of peak intervals determined between signal peaks identified at block 352 as described above. In some examples, the fundamental frequency may be determined as a trimmed mean, e.g., by removing one or more longest and/or one or more of the shortest peak intervals between the signal peaks identified from the rectified difference signal determined from the cardiac signal segment. The SW may then be determined by control circuit 80 by subtracting the MP from the fundamental period.

In some examples, control circuit 80 determines when VT/VF criteria are met based on the SW and the MP. Control circuit 80 may compare the ratio of SW to MP to a threshold in some examples. When the MP is at least 60 ms and the SW/MP ratio is less than or equal to 0.1, for example, the cardiac signal segment may be classified as a VT/VF segment at block 364. In another example, when the MP is at least 65 and the SW/MP ratio is less than or equal to a variable threshold that may be defined as a function of the MP, the cardiac signal segment may be classified as VT/VF at block 364. In an illustrative example, the SW/MP ratio may be compared to a linear function of the MP given by the equation {−0.0035*(MP)+0.145}. When the SW/MP ratio is less than or equal to this variable threshold determined as a function of the MP, the cardiac signal segment may be classified as a VT/VF segment at block 364. Other coefficients and constants may be used to define a variable threshold as a function of a morphology metric, e.g., MP, that is applied to another morphology metric or mathematical relationship of two or more morphology metrics, e.g., SW/MP ratio.

A variable threshold defined as a function of a morphology metric may be tailored to an individual patient or optimized based on data from a population of patients for classifying cardiac signal segments as VT/VF with high sensitivity and/or specificity. Other thresholds may be defined depending on the time duration of the cardiac signal segment for reliably discriminating between the morphology metrics of a VT/VF segment and a non-VT/VF segment based on MP and the SW/MP ratio.

The VT/VF criteria applied to the morphology metrics at block 360 may depend on the duration of the cardiac signal segment and/or the tachyarrhythmia operating state of ICD 14. In one example, when ICD 14 is operating in the unconcerned sensing state 1 and the GMA is triggered in response to detection of a suspected long pause or detection of suspected VT/VF undersensing, control circuit 80 may apply thresholds to the MP and the SW/MP ratio that are different than the thresholds applied during the concerned state 2 of the tachyarrhythmia detection states. For instance, when ICD 14 is operating in unconcerned state 1, control circuit 80 may determine that VT/VF criteria are met at block 360 when the MP is at least 60 and the SW/MP ratio is less than or equal to 0.1. When ICD 14 is operating in the concerned state 2 (or other higher tachyarrhythmia operating state), control circuit 80 may determine that VT/VF criteria are met at block 360 when the MP is at least 65 and the SW/MP ratio is less than or equal to −0.0035*MP+0.145. When the MP and SW do not meet the VT/VF criteria applied at block 360, the cardiac signal segment may be classified as non-VT/VF at block 362.

As described below in conjunction with FIG. 14, during post-shock pacing (or bradycardia pacing following a long pause detection), GMA may be performed using relatively shorter cardiac signal segments, e.g., 0.5 second segments, that can be acquired between delivered pacing pulses. When the cardiac signal segment is only 0.5 seconds long instead of 3 seconds long, for example, control circuit 80 may classify the cardiac signal segment as a VT/VF segment when the MP is at least 60 and the SW/MP ratio is greater than −1 and less than or equal to 0.085, as an illustrative example. As such, while the process of flow chart 350 may be performed whenever a GMA result is needed according to tachyarrhythmia sensing methods and/or bradycardia sensing methods, the criteria applied to the morphology metrics determined from the cardiac signal segment may be different depending on the tachyarrhythmia operating state and/or the bradycardia operating state of control circuit 80.

In other examples, a low slope content (LSC) and/or one or more noise metrics such as a muscle noise pulse count, mean rectified area, normalized mean rectified area may be determined by control circuit 80 as morphology metrics for determining when VT/VF criteria are satisfied at block 360. The low slope content may be determined by summing all of the sample points of the rectified difference signal that have an amplitude that is less than or equal to a low slope amplitude threshold and dividing the sum by the total number of sample points in the cardiac signal segment.

In some examples, LSC, MP, and/or SW may each be compared to respective thresholds for classifying the cardiac signal segment as a VT/VF segment or non-VT/VF segment. In an illustrative example, when the MP is at least 60 ms, the SW is less than or equal to 10 ms and the LSC is less than or equal to 0.7(70%), control circuit 80 may determine that the cardiac signal segment is a VT/VF segment at block 364. When the MP is less than 60 ms or the SW is greater than 10 ms or the LSC is greater than 0.7, control circuit 80 may determine that the cardiac signal segment is a non-VT/VF segment at block 362.

In some examples, control circuit 80 may determine a heart rate estimate as a morphology metric at block 360. A peak interval metric may be determined as a mean, median or other representative value of peak intervals determined between peaks of signal pulses identified at block 352 as described above. The peak interval metric may be correlated to the rate of any ventricular event signals in the cardiac signal segment and provide evidence for classifying the cardiac signal segment as VT/VF without requiring or in the absence of Vsense signals from sensing circuit 86. In some examples, control circuit 80 may determine a count of the signal peaks identified in the cardiac signal segment. The identified signal peaks are at least a threshold time interval apart based on the methods for identifying signal peaks described above in conjunction with block 352. Based on the time duration of the cardiac signal segment, a count of the identified signal peaks can be an indication of the average rate of signal pulses during the cardiac signal segment. A heart rate estimate based on the peak interval metric or the identified signal pulse count may be compared to VT/VF rate criteria at block 360 for classifying the cardiac signal segment as VT/VF or non-VT/VF in some examples.

The combination of MP and SW/MP ratio as morphology metrics used for discriminating between VT/VF segments and non-VT/VF segments can reliably classify VF segments with a high sensitivity without requiring determining LSC, heart rate estimate metrics or noise metrics from the cardiac signal segment. It is recognized, however, that numerous criteria may be conceived for discriminating between VT/VF segments and non-VT/VF segments using a variety of combinations of LSC, MP, SW, one or more heart rate estimate metrics, and/or one or more noise metrics. Such combinations may include mathematical combinations of two or more metrics (e.g., SW/MP ratio). VT/VF criteria applied by control circuit 80 at block 360 may include one or more thresholds applied to a respective metric or mathematical combination of metrics where each threshold can be defined as a constant or as a function, e.g., a linear function, of a metric determined from the cardiac signal segment. Other examples of performing a GMA for classifying a cardiac signal segment as being asystole, VT/VF or non-VT/VF are generally disclosed in U.S. Patent No. Ser. No. 18/045,135 (Aranda Hernandez, et al.), filed on Oct. 7, 2022, the entire contents of which is incorporated herein by reference and in the above-incorporated U.S. Patent. No. 63/310,558 (Heinks, et al.).

When the VT/VF criteria are met at block 360, the cardiac signal segment is classified as a VT/VF segment at block 364. Control circuit 80 may restart a long pause detection interval to avoid cardiac pacing by therapy delivery circuit 84 which may interfere with or delay the detection of a VT/VF episode. Control circuit 80 may increase the sensitivity of sensing circuit 86, e.g., by adjusting the sensitivity of both sensing channels 83 and 85 to a lower or minimum voltage amplitude setting available in response to a VT/VF classification when control circuit 80 is operating in the unconcerned sensing state 1 (FIG. 6). Control circuit 80 may continue performing GMA on one or more subsequent cardiac signal segments until at least one cardiac signal segment is classified as asystole or non-VT/VF. If a cardiac signal segment is classified as VT/VF and is the Nth VT/VF segment required for forcing a transition to the concerned state 2 and at least one VTI/VFI counter has reached a threshold percentage of an NID, control circuit 80 may transition from the unconcerned state 1 to the concerned state 2 (see FIGS. 5 and 6). If the cardiac signal segment is classified as VT/VF at block 364 and is the Nth VT/VF segment classified as VT/VF when all VTI/VFI counters are less than a threshold value, control circuit 80 may transition from block 122 to block 120 of the unconcerned sensing state 1 (see FIG. 6) of the tachyarrhythmia operating states.

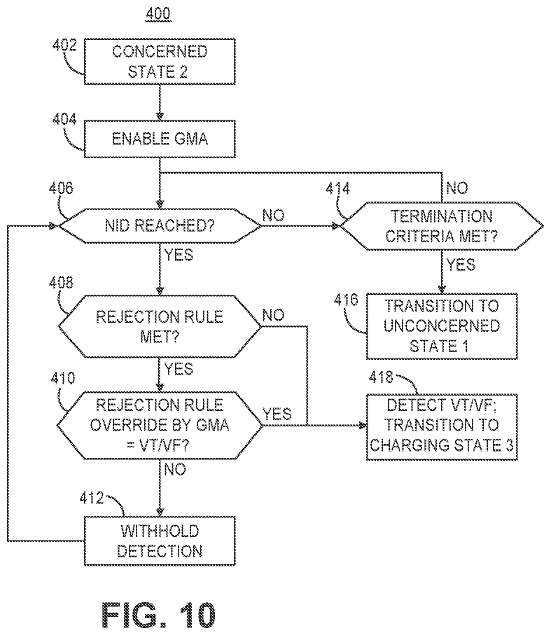

When the VT/VF criteria are not met at block 360, control circuit 80 may classify the cardiac signal segment as non-VT/VF. When the first GMA performed in response to suspected long pause detection results in a non-VT/VF or asystole classification, control circuit 80 may deliver at least one pacing pulse when a long pause is confirmed. When the first GMA performed in response to detecting suspected VT/VF undersensing results in a non-VT/VF classification, sensing circuit 86 may return to block 120 from block 124 of the unconcerned sensing state 102 of FIG. 6. Sensing circuit 86 may continue sensing ventricular event signals according to the programmed sensitivity of each sensing channel 83 and 85. The GMA may be disabled unless needed for other bradycardia or tachyarrhythmia sensing purposes. These and other responses to a cardiac signal segment classification based on the GMA performed according to flow chart 350 are further described above in conjunction with FIG. 6 and below in conjunction with FIGS. 10-14.

FIG. 10 is a flow chart 400 of a method for detecting VT/VF that may be performed by ICD 14 during the concerned state 2 of the tachyarrhythmia operating states described above in conjunction with FIG. 5, according to some examples. At block 402, control circuit 80 transitions to the concerned tachyarrhythmia suspected state 2 when any of the transition criteria described above in conjunction with FIG. 5 are met. If cardiac signal segment buffering and GMA of cardiac signal segments are not already in progress, control circuit 80 may enable cardiac signal segment buffering and GMA at block 404. In some instances, the transition to the concerned state 2 occurs in response to an NID being met when the programmed sensitivity is in effect. In this case, GMA may not be enabled or in progress upon transitioning to concerned state 2 and is enabled at block 404.

In other instances, GMA may be enabled during the unconcerned state 1 as described above in conjunction with FIG. 6, e.g., due to suspected VT/VF undersensing as described in conjunction with FIG. 8 or due to a suspected long pause based on bradycardia sensing methods. When the transition to the concerned state 2 occurs when the ventricular sensitivity is increased (e.g., at block 122 as shown in FIG. 6 and described above), buffering of cardiac signal segments for GMA may already be enabled and may remain enabled upon transitioning to the concerned state 2.

At block 406, control circuit 80 determines if an NID is met by the sensing channel 83 or 85 that is selected for reliable sensing for VT/VF detection as described above in conjunction with FIG. 7. In some instances, the NID is met upon transition to the concerned state 2. In other instances, a percentage of the NID may be reached, but not the full value of the NID, when control circuit 80 transitions to the concerned state 2 if a threshold number of cardiac signal segments have been classified as VT/VF based on the GMA enabled during the unconcerned sensing state (see arrow 130 of FIG. 6). If the NID is not met, control circuit 206 may determine if termination criteria are met at block 414. When termination criteria are not yet met after transitioning to state 2, control circuit 80 returns to block 406 and waits for either the NID to be reached or termination criteria to be met, whichever comes first.

If the termination criteria are met at block 414, e.g., according to any of the examples described above in conjunction with FIG. 5, control circuit 80 can transition back to the unconcerned state 1 at block 416. Upon transitioning back to the unconcerned sensing state 1, the sensitivity may be restored to the programmed sensitivity (if previously increased), and the GMA can be disabled. The VTI/VFI counters may be reset to zero. Control circuit 80 may begin counting VT/VF intervals based on Vsense signals received from both sensing channels 83 and 85, e.g., according to the methods performed at block 120 of FIG. 6.

If the NID is reached at block 406 before termination criteria are met at block 414, control circuit 80 may determine if a rejection rule is met at block 408. When the NID is reached causing the transition to the concerned state 2, control circuit 80 may advance direction to block 408 to determine if a rejection rule is met. As generally described above in conjunction with FIG. 7, control circuit 80 may determine sensed event data corresponding to Vsense signals received from the selected sensing channel. Sensed event data may continue to be determined and stored for use in determining if a rejection rule is met at block 408. One or more rejection rules may be applied to sensed event data for determining when oversensing of cardiac signals (e.g., P-waves or T-waves falsely sensed as R-waves), oversensing of non-cardiac signals (e.g., skeletal muscle electrophysiological signal pulses or electromagnetic interference falsely sensed as R-waves), and/or a conducted SVT may be present. When a rejection rule is met based on various criteria applied to the sensed event data, a VT/VF detection based on the NID being met can be withheld. Examples of methods that may be performed by control circuit 80 for determining when a rejection rule is met are described below, e.g., in conjunction with the flow charts of FIGS. 11 and 12.

When the NID is met and a rejection rule is not met (“no” branch of block 408), control circuit 80 may advance to block 418. Control circuit 80 may detect VT/VF and transition to the charging state 3 (shown in FIG. 5). If control circuit 80 determines that a rejection rule is met at block 408, control circuit 80 may determine whether the rejection rule being met is overridden by a GMA result being VT/VF at block 410. A rejection rule for withholding a VT/VF detection may be overridden when a GMA result is VT/VF such that control circuit 80 may still detect VT/VF. In some examples, multiple rejection rules may be applied by control circuit 80, such as a T-wave oversensing rejection rule, a P-wave oversensing rejection rule, one or more noise rejection rules (e.g., for rejecting oversensing of EMI or skeletal muscle noise pulses) and/or one or more SVT rejection rules (e.g., for rejecting sinus tachycardia or rapidly conducted atrial fibrillation). One or more rejection rules may be met or “fire,” e.g., when the respective rejection rule is determined to be true based on criteria applied to sensed event data by control circuit 80. Some, but not necessarily all, rejection rules may be overridden by a GMA result that is VT/VF. As such, in some cases, a rejection rule that fires may take precedence over the NID being met and a GMA result of VT/VF such that VT/VF detection is withheld at block 412. In other cases, when a rejection rule that fires can be overridden by a GMA result, and the current or the most recent GMA result is VT/VF, control circuit 80 may disregard or ignore the rejection rule. Control circuit 80 may detect VT/VF at block 418.

One or more rejection rules applied at block 408 may be overridden by a GMA result of VT/VF when the rejection rule is met. As such, if a GMA result is available at the time that the NID is met and the rejection rule is determined to be met, control circuit 80 may determine if the GMA result is VT/VF. The GMA result may be required to be less than 0.5 seconds old, 0.75 seconds old, or 1 second old in various examples. For instance, if the GMA result was determined more than 1 second earlier than the rejection rule fired, the GMA result may not be used to override the rejection rule. VT/VF detection may be withheld at block 412 and control circuit 80 may return to block 406. If the GMA result is determined within 1 second (or other specified time interval) prior to the rejection rule firing, control circuit 80 may use the GMA result of VT/VF to override the rejection rule. The GMA result of VT/VF in combination with the NID being reached may cause control circuit 80 to detect VT/VF at block 418 and transition to the charging state 3, even if a rejection rule has been met.

In some examples, a GMA result may not be available at the time that the rejection rule fires. In some examples, in order to avoid an unnecessary CV/DF shock, control circuit 80 may wait for buffering of a cardiac signal segment and the processing required for classifying the cardiac signal segment as VT/VF, non-VT/VF or asystole to be completed. If a GMA result is not available at the time that the rejection rule is met, therefore, control circuit 80 may withhold the VT/VF detection at block 412 and return to block 406. Control circuit 80 may continue to update VTI/VFI counters for a determination of whether the NID is met or not with each received Vsense signal. Control circuit 80 may apply termination criteria to RRIs with each received Vsense signal. Control circuit 80 can continue to determine sensed event data and any other signal analysis as required for updating the status of the rejection rule(s) applied at block 408.

If the NID is still met and the rejection rule is still met at block 408 when the GMA result becomes available, control circuit 80 may continue to withhold the VT/VF detection at block 412 if the cardiac signal segment is classified as asystole or non-VT/VF based on the GMA. Control circuit 80 may return to block 406. If, however, the cardiac signal segment is classified as VT/VF based on the GMA, the rejection rule may be overridden. Control circuit 80 may detect VT/VF at block 418 and transition to the charging state 3 in response to the NID being met and the VT/VF classification of the cardiac signal segment. It is to be understood that, at any time during the process of performing the GMA and determining if a rejection rule is met, control circuit 80 may determine that termination criteria are met (block 414) and return to the unconcerned state 1 before detecting VT/VF.

FIG. 11 is a flow chart 450 of a method that may be performed by control circuit 80 for determining when a cardiac event oversensing (OS) rejection rule is met according to some examples. In this example, the cardiac event oversensing rejection rule being applied is a P-wave oversensing (PWOS) rejection rule. PWOS occurs when a P-wave of the cardiac electrical signal crosses the R-wave sensing threshold, causing the selected sensing channel 83 or 85 to produce a false Vsense signal that is passed to control circuit 80. It is to be understood, however, that the techniques for detecting cardiac event OS described in conjunction with FIG. 11 could be applied to other types of oversensing, e.g., T-wave oversensing.

At block 452, control circuit 80 receives a Vsense signal from the sensing channel selected as the reliable sensing channel for VT/VF detection (e.g., as described on conjunction with FIG. 7). At block 454, control circuit 80 may determine one or more signal features from a sensed event signal segment buffered in memory 82 in response to the Vsense signal. The sensed event data may include features determined from the cardiac electrical signal sensed by the sensing channel 83 or 85 selected for VT/VF detection upon transitioning to the concerned state 2. For example, the maximum peak of the rectified cardiac electrical signal following an R-wave sensing threshold crossing and/or the peak-to-peak amplitude of the selected sensing channel signal during an R-wave peak tracking period or post-sense blanking period may be determined by sensing circuit 86 or control circuit 80 following the R-wave sensing threshold crossing corresponding to the Vsense signal. The sensed event data may include the RRI determined for the received Vsense signal.

The sensed event data determined at block 454 may include features determined from the morphology signal received from morphology signal channel 87. For example, the R-wave morphology matching score may be determined by control circuit 80 and stored with sensed event data for the Vsense signal. The sensed event data may be buffered in memory 82, e.g., for at least 3 to 24 of the most recent Vsense signals, so that the sensed event data is available for determining when a rejection rule is met. It is contemplated that sensed event data buffered in memory 82 for determining if a rejection rule is met in response to an NID being reached may be determined for Vsense signals received from the selected sensing channel 83 or 85 before transitioning to the concerned state 2 and/or after transitioning to the concerned state 2.

Other examples of sensed event data that may be buffered for determining if a PWOS rejection rule is met may include the polarity (positive or negative) of the maximum absolute peak amplitude. In still other examples, a signal feature determined at block 454 may be a maximum slope, total area (e.g., integral or summation of the sample point amplitudes), or other feature of a sensed event signal segment received from morphology signal channel 87. Any signal feature that is expected to alternate due to alternating R-waves and oversensed P-waves when the Vsense signals produced by the selected sensing channel 83 or 85 correspond to an alternating pattern of R-waves and P-waves (e.g., P-R-Por R-P-R) may be determined and stored as sensed event data for consecutive Vsense signals. Two or more signal features may be determined from each sensed event signal segment to detect an alternating pattern of the combination of two or more signal features in some examples.

At block 456, control circuit 80 may compare the sensed event data determined for consecutively received Vsense signals, e.g., three consecutively received Vsense signals, to each other for detecting an alternating pattern of signal features. The sensed event data may be additionally compared to criteria for identifying true R-waves among the oversensed P-waves. For example, an alternating pattern of relatively high and relatively low maximum peak amplitudes may be detected at block 456. The peak amplitude may be the maximum peak of the rectified signal passed to the R-wave detector (66a or 66b in FIG. 4) determined by sensing circuit 86 during the R-wave peak tracking period. In other examples, the peak amplitude may be the maximum peak-to-peak amplitude of a non-rectified signal sensed by the sensing circuit 86, e.g., by the selected sensing channel 83 or 85 or by morphology signal channel 87, during a post-sense blanking period following a Vsense signal.

For the sake of illustration, the maximum peak amplitudes determined for three consecutive Vsense signals may be referred to as A1, A2 and A3. A3 can be the peak amplitude corresponding to the most recent (e.g., ith) Vsense signal. A2 can be the peak amplitude of the most recent preceding (e.g., i−1) Vsense signal. Al can be the peak amplitude of the i−2 Vsense signal. An alternating pattern of maximum peak amplitudes may be determined by control circuit 80 by determining a first amplitude difference D1 between amplitude A1 and amplitude A2, e.g., D1=A2−A1. A second amplitude difference D2 can be determined between the second amplitude A2 and the third amplitude A3, e.g., D2=A3−A2. The first and second amplitude differences D1 and D2 between the three consecutively determined maximum peak amplitudes A1, A2 and A3 may be compared to difference thresholds to detect an alternating pattern of high-low-high or low-high-low maximum peak amplitudes associated with the Vsense signals.

The difference thresholds may be a fixed value or set based on at least one of the determined A1, A2 or A3 peak amplitudes. In some examples, the difference threshold may be set as a percentage of one of the two amplitudes being compared (e.g., a percentage of A1 or A2 for comparison to D1 and a percentage of one of A2 or A3 for comparison to D2). The percentage of the peak amplitude applied by control circuit 80 as a difference threshold may be 15 to 35% of one of the two peak amplitudes being compared. In some examples, the difference threshold is 22% to 25% of one of the two peak amplitudes being compared.

In an illustrative example, control circuit 80 may compare D1 to 25% of A2 and compare D1 to 25% of A1. Control circuit 80 may compare D2 to 25% of A3 and to 25% of A2. Control circuit 80 may detect a high-low-high alternating pattern of three consecutive peak amplitudes when the negative of difference D1 (A2-A1) is greater than 25% of A1 and the difference D2 (A3-A2) is greater than 25% of A3. Control circuit 80 may detect a low-high-low alternating pattern of the three consecutive peak amplitudes when the difference D1 (A2-A1) is greater than 25% of A2 and the negative of the difference D2 (A3-A2) is greater than 25% of A2. It is recognized that other criteria may be conceived and applied for detecting an alternating pattern of high-low-high or low-high-low peak amplitudes of three consecutively sensed event signals.

Detecting an alternating pattern of high-low-high or low-high-low peak amplitudes that may correspond to PWOS may further require that sensed event signal segments buffered in response to the peak amplitudes identified as high amplitude events based on the comparative analysis of D1 and D2 have an R-wave morphology matching score that is greater than a match threshold. For example, when the amplitude criteria applied to D1 and D2 indicate a high-low-high pattern, control circuit 80 may determine if the R-wave morphology matching score determined for the first Vsense signal (corresponding to A1) and/or the third, most recent Vsense signal (corresponding to A3) are greater than a match threshold. The R-wave morphology matching score (also referred to herein as “morphology matching score”) may be determined between the sensed event signal segment buffered from morphology signal channel 87 and an R-wave template stored in memory 82 as generally described above in conjunction with FIG. 8. When the amplitude criteria applied to D1 and D2 indicate a low-high-low pattern, control circuit 80 may determine if the R-wave morphology matching score determined for the second Vsense signal (corresponding to A2) is greater than a match threshold. The match threshold may be 40%, 50%, 60%, 70% or other specified threshold. Additionally or alternatively, the morphology match scores stored for the Vsense signals corresponding to low peak amplitudes (A2 in the high-low-high or A1 and/or A3 in the low-high-low pattern) may be required to be less than a match threshold, e.g., less than 10%, 20%, 25%, 30%, 35% or 40%.

In other examples, an alternating pattern of event signal polarity may be detected at block 456. In still other examples, an RRI pattern of long-short when the amplitude pattern is high-low-high or an RRI pattern of short-long when the amplitude pattern is low-high-low may be required by the alternating pattern detection criteria applied by control circuit 80 at block 456. In yet another example, a pattern of low-high-low or high-low-high maximum slope of the sensed event signal may be required for detecting an alternating pattern of cardiac event signals at block 456. Control circuit 80 is described as analyzing three consecutively sensed event signals for detecting an alternating pattern of signal features in a high-low-high or low-high-low pattern in the illustrative examples described here. It is contemplated, however, that two consecutively sensed event signals for detecting low-high or high-low patterns could be analyzed, or more than three consecutively sensed event signals may be analyzed for detecting an alternating pattern (e.g., high-low-high-low or low-high-low-high, etc.).

Other sensed event data that may be determined at block 454 for a given Vsense signal may include a normalized rectified amplitude and/or a maximum pulse width of the sensed event signal segment received from the morphology signal channel 87. The normalized rectified amplitude and/or pulse width may be compared to criteria for identifying a sensed event signal waveform as being a true VT/VF waveform and not an oversensed P-wave. In some examples, the alternating pattern of sensed event data is not detected at block 456 when the normalized rectified amplitude and/or maximum pulse width meet true VT/VF waveform criteria.

In the example of FIG. 11, if either amplitude criteria or the R-wave morphology matching criteria for detecting an alternating pattern of sensed event signals are not met at block 456, control circuit 80 may return to block 452 to wait for the next Vsense signal. The PWOS rejection rule may remain unsatisfied. When control circuit 80 detects a high-low-high or low-high-low alternating pattern of sensed event signals at block 456 based on the peak amplitude analysis and R-wave morphology matching score analysis, control circuit 80 may detect evidence of PWOS based on the alternating pattern. However, control circuit 80 may determine if the most recent GMA result is VT/VF at block 460. If the most recent GMA result is VT/VF, this GMA result may override the PWOS rejection rule. In this example, control circuit 80 may return to block 452 (“yes” branch of block 460) without increasing an OS evidence counter at block 464. The PWOS rejection rule may remain unmet due to the GMA result.

When the most recent GMA result is not VT/VF, e.g., when the most recent cardiac signal segment is classified as asystole or non-VT/VF, control circuit 80 may increase an OS evidence counter at block 464 based on the detection of the alternating pattern of sensed event signals at block 456. Control circuit 80 may increase the OS cardiac event evidence counter by one or flag the most recent Vsense event signal and corresponding sensed event data as PWOS evidence to enable control circuit 80 to track the number of times an alternating pattern of cardiac event oversensing is identified without being overridden by a GMA result being VT/VF.

At block 466, control circuit 80 may compare the value of the OS evidence counter to a threshold value for detecting cardiac event oversensing, in this case PWOS. If the OS evidence counter has reached a threshold value, which means that at least the most recent GMA result is not VT/VF, the OS rejection rule is met, or “fires,” at block 470. The threshold applied to the OS evidence counter may be 3, 4, 5 or 6 out of the most recent 8, 10, 12, 20 or 24 Vsense signals, for example. In one example, when 3 out of 12 of the most recent Vsense signals are flagged as being evidence of PWOS, control circuit 80 may determine that the OS evidence counter has met the threshold at block 466, and the PWOS rejection rule is met at block 470 without being overridden based on the GMA. If a VTI/VFI counter associated with the selected sensing channel 83 or 85 has reached an NID, the VT/VF detection may be withheld in response to the PWOS rejection rule being met and not overridden based on the most recent GMA.

If the OS evidence count has not reached the threshold value at block 466, control circuit 80 may determine that the OS rejection rule is not met at block 468. Control circuit 80 may return to block 452 to wait for the next Vsense signal. In this example, the GMA result of VT/VF may be used to override the PWOS rejection rule by not counting or flagging a Vsense signal as OS evidence when the alternating pattern is detected and morphology matching criteria are met for the high amplitude sensed event signals. In this way, a current or most recent GMA result of VT/VF overrides the PWOS rejection rule by preventing the PWOS rejection rule from being met (OS evidence counter is not increased). In other examples, Vsense signals may be counted as OS evidence as the alternating patterns of cardiac event signals are detected, but, once the OS evidence count has reached a threshold value, control circuit 80 may determine that the PWOS rejection rule is overridden based on the most recent GMA result being VT/VF. It is to be understood therefore, that a GMA result of VT/VF may override a rejection rule by preventing the rejection rule from becoming met (as shown in the example of FIG. 11), or by causing control circuit 80 to disregard a rejection rule that is met when the current GMA result is VT/VF (as generally described above in conjunction with FIG. 10). Either way, if the NID is reached and the most recent GMA result is VT/VF, control circuit 80 may detect VT/VF to enable ATP or shock delivery without withholding or delaying the detection due to the PWOS rejection rule being met. PWOS may be occurring in the presence of a true VT/VF episode that can still be detected based on the GMA and NID being reached. However, when PWOS is detected according to the PWOS rejection rule being met and the most recent GMA result is not VT/VF, the PWOS rejection rule can cause control circuit 80 to withhold or delay the VT/VF detection.

Other PWOS rejection rules, T-wave oversensing (TWOS) rejection rules and/or non-cardiac noise oversensing rejection rules may be applied by control circuit 80 for determining when oversensing may be leading to the NID being falsely reached when VT/VF is not occurring. Other examples of PWOS rejection rules that may be applied by control circuit 80, which may be overridden by a GMA result of VT/VF as disclosed herein, are generally disclosed in U.S. Patent Application Publication No. 2021/0170170 (Mischler et al., filed Jun. 10, 2021).

Examples of TWOS rejection rules that may be applied by control circuit 80 (e.g., at block 408 of FIG. 10) are generally disclosed in U.S. Pat. No. 9,597,525 (Cao, et al., filed on May 6, 2015) and in U.S. Pat. No. 10,850,113 (Cao, et al., filed on July 20, 2017), both incorporated herein by reference in their entirety. For example, the peak amplitude of the sensed event signal and R-wave morphology matching scores determined as sensed event data for Vsense signals may be analyzed for determining when a TWOS rejection rule is met. For instance, TWOS evidence may be detected by control circuit 80 when a specified number of the most recent Vsense signals include a threshold number of pairs of Vsense signals that present a high-low or low-high amplitude pattern and present a corresponding high-low or low-high R-wave morphology matching score pattern. Control circuit 80 may determine that the TWOS rejection rule is met when TWOS evidence is detected a threshold number of times over the most recent 8, 10, 15, 20, 30 or other specified number of most recent Vsense signals. In some examples, when the TWOS rejection rule is met and a current GMA result is VT/VF, the TWOS rejection rule is overridden so that control circuit 80 may detect VT/VF. In other examples, a current GMA result of VT/VF may prevent a TWOS evidence counter from being increased, thereby overriding the TWOS rejection rule, when TWOS evidence is detected based on the high-low or low-high pattern of amplitudes and R-wave morphology matching scores.

Examples of noise rejection rules that may be applied by control circuit 80, e.g., at block 408 of FIG. 10, may include a skeletal muscle noise rejection rule and/or an electromagnetic interference (EMI) rejection rule. Noise rejection rules may be overridden by a GMA result of VT/VF according to the techniques disclosed herein. Examples of noise rejection rules that may be applied by control circuit 80 are generally disclosed in U.S. Pat. No. 10,470,681 (Greenhut et al., filed on May 26, 2017) and U.S. Pat. No. 10,561,332 (Zhang et al., filed July 19, 2017), both incorporated herein by reference in their entirety. The peak amplitudes determined as sensed event data may be used for detecting noise based on maximum and minimum peak amplitudes for discriminating noise pulses from ventricular event signals and/or for counting noise pulses. A normalized mean rectified amplitude and/or a maximum pulse width may be determined as sensed event data that can be used by control circuit 80 for discriminating between noise pulses and true ventricular sensed event signals for use in determining when a noise rejection rule is met. A sensed event signal identified as a noise signal based on sensed event data may cause a noise evidence counter to be increased toward a threshold value for satisfying a noise rejection rule. A current GMA result of VT/VF may override a noise rejection rule by preventing a noise evidence counter from being increased (so that the noise rejection rule is prevented from being met) or by causing control circuit 80 to disregard the noise rejection rule when it is met.

FIG. 12 is a flow chart 500 of a method that may be performed by control circuit 80 for determining when an SVT rejection rule is met according to some examples. The criteria required for the SVT rejection rule to be met in this example may correspond to atrial fibrillation (AF) being conducted to the ventricles. The rejection rule may be referred to as an AF rejection rule in this case. However other SVT rejection rules may be applied and/or the SVT rejection rule criteria described in conjunction with FIG. 12 may cause control circuit 80 to withhold a VT/VF detection when other types of SVT are occurring, including but not necessarily limited to AF conducted to the ventricles.

At block 502, control circuit 80 receives a Vsense signal from sensing circuit 86. At block 504, control circuit 80 may determine sensed event data corresponding to the Vsense signal. The sensed event data may be stored in memory 82 to enable control circuit 80 to determine when the sensed event data for a threshold number of Vsense signals satisfy the SVT rejection rule. As indicated above, sensed event data determined before and/or after transitioning to the concerned state 2 may be used for determining the status of a rejection rule. The sensed event data buffered for Vsense signals received from only the sensing channel 83 or 85 that is selected for VT/VF detection upon transitioning to the concerned state 2 may be analyzed by control circuit 80 for determining if a rejection rule is met.

As described above, the sensed event data may include the RRI ending with the currently received Vsense signal. The sensed event data may include an R-wave morphology matching score determined from a sensed event signal segment buffered in memory 82 from the morphology signal received from morphology signal channel 87 in response to the Vsense signal.

At block 506, control circuit 80 may update an RRI mode from the sensed event data. The RRI mode may be the most frequently occurring RRI, or a range of the most frequently occurring RRIs, out of the RRIs buffered for the most recent 12 to 30 RRIs. The RRI mode may be the most common RRI determined out of the most recent 24 RRIs in some examples. Control circuit 80 may track the frequency of RRIs occurring over multiple RRI ranges in a histogram allocated in memory 82. For example, control circuit 80 may log RRIs between 150 ms and 1 second or between 150 ms and 600 ms, as examples, in the RRI histogram. The histogram may be divided into 10 ms bin widths in an example. The histogram bins may have a bin width of 5 ms to 30 ms in other examples. The RRI mode may be identified as the histogram bin having the greatest number of RRIs logged for the corresponding RRI bin range. In some examples, the two most frequently occurring RRI ranges as defined by the histogram bin widths during the most recent 24 RRIs (or other specified number of RRIs) are determined at block 506. In still other examples, three or more of the most frequently occurring RRIs may be determined at block 506. The one, two or more most frequently occurring RRIs may be identified as the RRI bins having the highest counts of RRIs logged in the respective bins over the most recent 16 to 30 RRIs, for example. In other examples, an RRI mode may be determined from all buffered RRIs, and a most frequent RRI range may be determined as being the RRI mode ±5 ms, 10 ms, 15 ms or other specified range from the RRI mode at block 506.

At block 508, control circuit 80 may determine if RRI regularity criteria are met. When AF is being conducted to the ventricles, the RRIs are expected to be irregular. Control circuit 80 may determine what percentage of all RRIs fall within the one, two or more RRI histogram bins (ranges) identified as the most frequently logged bins at block 506. In other examples, control circuit 80 determines what percentage of all RRIs fall within a specified range of the RRI mode. Control circuit 80 may determine if RRIs are regular by comparing the percentage of RRIs logged in the two most frequent RRI bins, each having a bin width of 10 ms, to a threshold percentage. If at least 80%, 90% or 100% of the most recent 24 RRIs fall within the two most commonly logged RRI bins, control circuit 80 may determine RRI regularity criteria are met at block 508. Control circuit 80 may return to block 502 to wait for the next Vsense signal. The SVT rejection rule, in this case an AF rejection rule, remains unmet such that if the NID is reached by a VTI/VFI counter and no other rejection rules are met, VT/VF may be detected by control circuit 80.

If less than a threshold percentage of the RRIs fall within the two most frequently logged histogram bins (or other most common RRI range identified at block 506), control circuit 80 may determine that the RRIs are irregular. The RRI regularity criteria at block 508 are not met, which may be an indication of conducted AF. In one example, if less than 100% of the most recent 24 RRIs fall within the two most frequently logged histogram bins, control circuit 80 determines that RRI regularity criteria are not met at block 508. During AF, depolarizations conducted from the atria to the ventricles are expected to occur at irregular intervals. RRI irregularity criteria required for the SVT rejection rule to be met can be satisfied when the RRI regularity criteria are not met at block 508. Control circuit 80 may advance to block 510 to apply R-wave morphology matching criteria.

Control circuit 80 may compare the R-wave morphology matching scores buffered with the sensed event data to an SVT match threshold. The SVT match threshold may be defined differently than a match threshold applied to R-wave morphology matching scores for other purposes, such as determining when the PWOS rejection rule is met as described above in conjunction with FIG. 11. The SVT match threshold may be relatively lower, e.g., less than the match threshold used for identifying true R-waves in a high-low-high or low-high-low alternating pattern of sensed event signals. The SVT match threshold may be 10% to 50% in various examples and is 10% to 30% in some examples. If at least a threshold percentage of the most recent morphology matching scores meet the SVT match threshold, control circuit 80 may determine evidence of an SVT. For example, control circuit 80 may identify evidence of conducted AF when the RRI regularity criteria are not met (“no” branch of block 508) and at least 18 out of the most recent 24 morphology matching scores are at least 10%, 15%, 20% or 25% as examples. It is to be understood that the sensed event data buffered for another specified number of the most recent Vsense events may be analyzed and/or different thresholds than the example thresholds provided here may be used in determining when RRI irregularity and R-wave morphology matching scores satisfy an SVT rejection rule.

When the RRI regularity criteria are not met and the SVT morphology matching criteria are met at the respective blocks 508 and 510, control circuit 80 may determine if the most recent GMA result is VT/VF. If so, the VT/VF classification of the cardiac signal segment may override the evidence of an SVT. If the most recent GMA result is VT/VF, control circuit 80 may override the SVT rejection rule by determining that the SVT rejection rule is not met at block 514. In other examples, control circuit 80 may determine that the SVT rejection rule is met based on the RRI regularity criteria not being met at block 508 and the threshold number of R-wave morphology matching scores meeting the SVT match threshold at blocks 508 and 510. The SVT rejection rule, however, may be overridden by the GMA result being VT/VF so that if an NID is reached, VT/VF is not withheld based on the SVT rejection rule which is ignored or disregarded when the GMA result is VT/VF.

When the most recent GMA result is not VT/VF (e.g., when the GMA result is non-VT/VF or asystole), and the RRI regularity criteria are not met at block 508 and the SVT morphology matching criteria are met at block 510, control circuit 80 may determine that the SVT rejection rule is met at block 516. If an NID is reached by a VTI/VFI counter of the selected sensing channel 83 or 85, control circuit 80 may withhold a VT/VF detection based on the SVT rejection rule being met and not overridden by the GMA.

Other SVT rejection rules may be applied during the concerned state 2, charging state 3, and/or redetection state 5. Each SVT rejection rule may be overridden by a GMA result of VT/VF when the rejection rule is met. Each SVT rejection rule may be applied by control circuit 80 by applying various criteria to sensed event data, e.g., RRIs, R-wave morphology matching scores, and/or signal features derived from the sensed event signal segment for identifying sensed event signals characterized by RRIs and/or a morphology that has a higher likelihood of arising in the atria and being conducted to the ventricles than being a VT/VF signal waveform. A peak polarity pattern of the sensed event signal, a peak time interval extending from a maximum positive peak to a minimum negative peak of the sensed event signal, a normalized width of the sensed event signal, and an R-wave morphology matching score are examples of sensed event data that may be determined from a sensed event signal segment for use in determining if an SVT rejection rule is met by control circuit 80. Other examples of SVT rejection rules that can be applied for withholding a VT/VF detection when an NID is reached are generally disclosed in U.S. U.S. Pat. No. 10,555,684 (Zhang et al.), incorporated herein by reference in its entirety.

FIG. 13 is a flow chart 550 of a method that may be performed by control circuit 80 for redetecting VT/VF while operating in the redetection state 5 of the tachyarrhythmia operating states shown in FIG. 5 according to some examples. Control circuit 80 transitions to the redetection state 5 at block 551. Control circuit 80 may transition to the redetection state 5 from therapy delivery state 4 after delivery of a CV/DF shock. In other examples, control circuit 80 may transition to the redetection state 5 in response to abort therapy criteria being met after VT/VF is detected. Control circuit 80 may determine that abort therapy criteria are met while operating in charging state 3 (block 106 of FIG. 5) or while operating in the therapy delivery state 4 (block 108 of FIG. 5), e.g., during capacitor charging or while synchronizing ATP or shock delivery with the patient's heart rhythm, up until the time that ATP or a CV/DF shock is delivered.

When control circuit 80 transitions to redetection state 5 after therapy delivery, control circuit 80 may begin buffering a new cardiac signal segment for performing GMA. When control circuit 80 transitions to redetection state 5 in response to abort therapy criteria being met, cardiac signal segment buffering or processing for performing the GMA may be underway and is not necessarily restarted upon transitioning to the redetection state 5. GMA remains enabled, and the sensing channel 83 or 85 selected for reliable VT/VF detection and the programmed or increased sensitivity in effect at the time of initial VT/VF detection may remain in effect during redetection state 5.

Upon transitioning to the redetection state 5 following delivery of a CV/DF shock, control circuit 80 may start a long pause confirmation interval, referred to hereafter as a “pause confirmation interval,” for detecting post-shock asystole. If control circuit 80 has transitioned to the redetection state 5 in response to abort therapy criteria being met, control circuit 80 may start a relatively longer hysteresis pacing interval during which bradycardia sensing methods may be performed. The relatively long hysteresis pacing interval can be started to avoid bradycardia pacing pulses from interfering with redetection of the VT/VF after the abort therapy criteria are met. The relatively shorter pause confirmation interval may be started following a CV/DF shock to enable post-shock pacing to begin without an undue delay. The pause confirmation interval may be 2 to 4 seconds or 2.75 to 3.75 seconds and is 3.0 to 3.5 seconds in an example. The relatively longer hysteresis pacing interval, which may include a long pause detection interval followed by the pause confirmation interval, may be 4 to 9 seconds or 5 to 8 seconds and is 5.5 to 6.5 seconds or about 6 seconds in some examples. Bradycardia sensing and pacing control methods generally disclosed in U.S. patent application Ser. No. 17/822,681 (Greenhut, et al., filed Aug. 26, 2022), incorporated herein by reference in its entirety, and in the above-incorporated provisional U.S. Patent Application No. 63/486,725 (Greenhut, et al., filed February 24, 2023) may be combined with the techniques disclosed herein.

Control circuit 80 may wait for a Vsense signal from the selected sensing channel 83 or 85 at block 552. If a Vsense signal is not received before the pause confirmation interval expires, as determined at block 554, control circuit 80 may determine at block 556 if a cardiac signal segment is classified as VT/VF based on the GMA. Upon starting the pause confirmation interval, buffering a cardiac signal segment for GMA may begin so that a GMA result is available upon expiration of the pause confirmation interval. If the GMA result is VT/VF (“yes” branch of block 556), control circuit 80 may restart the pause confirmation interval at block 568. Post-shock pacing is not delivered if the GMA result is VT/VF to avoid pacing pulse delivery from interfering with redetection of VT/VF.

If the GMA result is not VT/VF, control circuit 80 may control therapy delivery circuit 84 to deliver at least one pacing pulse at block 560. Control circuit 80 may start a post-shock pacing period at block 560. A post-shock pacing period may be started at block 560 that is a specified time duration, e.g., 10 seconds, 20 seconds or 30 seconds. Post-shock pacing may be delivered by therapy delivery circuit 84 at a programmed post-shock pacing rate during the post-shock pacing period. The post shock pacing rate may be 40, 50, or 60 pulses per minute as examples. However, if a Vsense signal is received from sensing circuit 86 (from the selected sensing channel 83 or 85), a scheduled pacing pulse may be inhibited by therapy delivery circuit 84. Control circuit 80 may control therapy delivery circuit 84 to deliver the post-shock pacing in a VVI pacing mode so that Vsense signals inhibit a pacing pulse and restart the pacing escape interval for scheduling the next pacing pulse. Therapy delivery circuit 84 may deliver VVI pacing for the post-shock pacing period or until a GMA result is VT/VF, which may terminate the post-shock pacing period.

Control circuit 80 may advance to block 562 (from block 554, 558 or 560) to determine if termination criteria are met. It is to be understood that control circuit 80 may be applying termination criteria to the Vsense signals being received from sensing circuit 86 during a pause confirmation interval started upon transitioning to the redetection state 5. Termination may be detected according to any of the examples given above. For instance, control circuit 80 may detect termination of VT/VF (following a delivered or aborted therapy) when at least eight Vsense signals are received at RRIs that meet a slow interval threshold (e.g., the RCL plus 60 ms or another specified offset). Additionally or alternatively, control circuit 80 may detect termination of VT/VF at block 562 when the median RRI determined from a specified number of most recent RRIs (e.g., 6 to 12 RRIs) is longer than a VT/VF detection interval for a specified time interval, e.g., for at least 10 to 20 seconds. In an example, when the median RRI determined from the most recent 8 RRIs is persistently longer than the programmed VT detection interval for at least 20 seconds, control circuit 80 may detect termination at block 562. Control circuit 80 may transition to the unconcerned sensing state at block 564 in response to detecting VT/VF termination.

When termination criteria are not met, control circuit 80 may wait for the next Vsense signal at block 552. If the post-shock pacing period has been started at block 560, a Vsense signal received at block 552 causes therapy delivery circuit 84 to inhibit a scheduled ventricular pacing pulse. If a Vsense signal is not received before the pacing interval expires, therapy delivery circuit 84 may deliver the scheduled pacing pulse. While not shown explicitly in FIG. 13, it is to be understood that if the post-shock pacing period expires and control circuit 80 is still operating in the redetection state 5, control circuit 80 may restart the pause confirmation interval. However, if the post-shock pacing period is 10 seconds or more, e.g., 30 seconds, it is likely that termination or redetection of the VT/VF may be detected before the post-shock pacing period expires.

In response to a Vsense signal received at block 552, control circuit 80 determines sensed event data at block 570 for use applying termination criteria and redetection criteria, including any rejection rules that may be enabled during the redetection state 5. The sensed event data may include the sensed event signal peak amplitude (which may be a rectified maximum peak amplitude and/or a maximum peak to peak amplitude), the RRI, and the R-wave morphology matching score. Other sensed event data described herein may be determined at block 570 as needed for detecting termination and for redetecting VT/VF. At block 572, control circuit 80 may determine if the RNID (redetection NID) is reached. If not, control circuit 80 may advance to block 562 to determine if termination criteria are met.

When the RNID is met by a VTI/VFI counter of the selected sensing channel 83 or 85 (block 572), control circuit 80 may apply one or more rejection rules at block 574 to discriminate between true VT/VF and oversensing of cardiac events, oversensing of noise, and/or SVT which may be causing the RNID to be reached based on Vsense signals. Control circuit 80 may apply at least some of the rejection rules that are applied during the concerned state 2 for detecting VT/VF. For example, the PWOS rejection rule as described above in conjunction with FIG. 11 may be applied during the redetection state 5. However, in some examples, at least some rejection rules applied during the concerned state 2 may be disabled or not applied during the redetection state 5. For example, an SVT rejection rule, such as the conducted AF rejection rule described above in conjunction with FIG. 12, may be disabled during the redetection state 5. The RRIs following a delivered CV/DF shock may be irregular, even during monomorphic VT. Regularity criteria applied to post-shock RRIs for determining if a conducted AF rejection rule is met may not be reliable for discriminating between a conducted AF and VT/VF after CV/DF shock delivery. As such, during redetection state 5, control circuit 80 may apply one or more rejection rules that are applied during the concerned sensing state 2 but may disable other rejection rules that are applied during the concerned sensing state 2.

When none of the applied rejection rules are met, as determined at block 574, control circuit 80 may redetect VT/VF at block 582 in response to the RNID being met. Control circuit 80 may transition back to the charging state 3 (as shown in FIG. 5). When a rejection rule is met at block 574, control circuit 80 may determine if a GMA result is available. The rejection rule may be overridden by a GMA result of VT/VF. In some cases, the RNID may be met and a rejection rule may be met before a GMA result is available, however.

The first GMA result may not be available for up to 3 to 4 seconds after transitioning to the redetection state depending on the duration of the cardiac signal segment being buffered and required GMA processing time. Furthermore, buffering of the cardiac signal segment for GMA may be delayed by a post-shock artifact delay period which may be 0.2 to 1 second or about 0.5 seconds in duration after the delivered CV/DF shock. In the example of a 3-second cardiac signal segment being buffered for GMA, the cardiac signal segment buffering and processing time for determining the GMA result may therefore not be available for at least 3.5 seconds after transitioning to the redetection state 5 in some examples.

In the example shown FIG. 13, if a rejection rule is met at block 574 and can be overridden by the GMA, but the GMA result is not yet available, control circuit 80 may withhold VT/VF redetection at block 580 based on the rejection rule being met. The VT/VF redetection may be withheld at least until the GMA result is available. If a rejection rule is met at block 574 and a GMA result is available (“yes” branch of block 576), e.g., determined within the last 0.5 to 1 second, control circuit 80 may advance to block 578. When the GMA result is a VT/VF classification of the cardiac signal segment, control circuit 80 may redetect VT/VF (block 582) based on the RNID being met and the GMA result of VT/VF. When the GMA result is available and is VT/VF, the rejection rule may be overridden. If, however, the GMA result is available and not VT/VF, e.g., if the cardiac signal segment is classified as non-VT/VF or asystole, control circuit 80 may withhold redetection of the VT/VF at block 580. Control circuit 80 may return to block 552 to wait for the next Vsense signal.

In other examples, however, when a rejection rule is met at block 574 and the GMA result is not available, but the RNID is reached by a VTI/VFI counter, control circuit 80 may designate the GMA result to be VT/VF by default. Control circuit 80 may override a rejection rule that is met based on a default GMA result of VT/VF when an RNID is met. Control circuit 80 may advance directly to block 582 to redetect VT/VF and transition to charging state 3 instead of waiting for the GMA result to become available even though a rejection rule is met. VT/VF may be redetected without waiting for the GMA result based on a higher likelihood of VT/VF still being present when the VT/VF was previously detected.

In some examples, multiple rejection rules may be applied during the redetection state 5. Any of the example rejection rules described herein may be applied at block 574. Each rejection rule can be applied to determine when a threshold number of Vsense signals (that are contributing to the NID being reached) are more likely to be oversensed signals or correspond to depolarizations conducted from the atria than true VT/VF R-waves or fibrillation waves. Any or all of the rejection rules applied, e.g., a TWOS rejection rule, a PWOS rejection rule, one or more SVT rejection rules, and/or one or more noise rejection rules, may be overridden by a GMA result of VT/VF in some examples. The criteria for applying a rejection rule may be highly sensitive for detecting oversensing (of P-waves, T-waves or non-cardiac noise) or detection of an SVT so that if the rejection rule is met, the GMA result can override a rejection rule that is falsely met. In this way, when PWOS, TWOS, an SVT or noise oversensing is causing an NID to be met, the PWOS, TWOS, SVT or noise oversensing can be detected with high sensitivity according to the rejection rule criteria, but VT/VF detection is only withheld if the GMA result confirms that a cardiac signal segment is not classified as VT/VF.

It is to be understood, however, in some cases a rejection rule that is applied during redetection state 5 may be a rejection rule that cannot be overridden by a GMA result. A rejection rule may be considered valid for withholding VT/VF redetection before a GMA result is available and/or when a GMA result is available but is not used for overriding the rejection rule. For example, a PWOS rejection rule, TWOS rejection rule, a noise rejection rule and/or other SVT rejection rules may be applied at block 574. If a rejection rule is met that cannot be overridden by the GMA result, control circuit 80 may withhold VT/VF redetection until the RNID is reached and all rejection rules are either unmet or overridden by the GMA result.

FIG. 14 is a diagram 600 of a method for performing GMA during a pacing period according to some examples. As described above, control circuit 80 may start a post-shock pacing period in response to a pause confirmation interval expiring without a Vsense signal being received and a GMA result being non-VT/VF or asystole. In order to promote redetection of VT/VF, control circuit 80 may determine sensed event data for Vsense signals received during (and after) the pacing period and may buffer and analyze cardiac signal segments according to the GMA for redetecting VT/VF until termination is detected or VT/VF is redetected, whichever occurs first. When post-shock pacing is not being delivered, a cardiac signal segment that is longer than one or more pacing escape intervals can be acquired without pacing artifact distorting the cardiac signal segment. For example, a 3-second cardiac signal segment can be buffered, independent of the timing of Vsense signals, for performing the GMA. However, when post-shock pacing is being delivered, multiple relatively shorter cardiac signal segments may be acquired during multiple pacing escape intervals to acquire a cumulative cardiac signal segment duration from which a VT/VF, asystole or non-VT/VF result can be determined according to the GMA of the multiple shorter cardiac signal segments. A method for performing GMA by control circuit 80 using cardiac signal segments buffered between post-shock pacing pulses (or bradycardia pacing pulses) is illustrated in FIG. 14.

In the example shown, therapy delivery circuit 84 may deliver a CV/DF shock 604 in response to a VT/VF detection. Control circuit 80 transitions to the redetection state 5 and may start the pause confirmation interval 620 after a post-shock delay interval 605 (to allow signal artifact to decay post-shock, prior to enabling sensing of ventricular event signals and buffering of the morphology signal from morphology sensing channel 87). The post-shock delay interval 605 may be 0.2 to 1 seconds long after delivery of the CV/DF shock 604.

Upon starting the pause confirmation interval 620, control circuit 80 may enable buffering of a cardiac signal segment from the morphology signal 602 received from morphology sensing channel 87 during the pause confirmation interval 620. When the pause confirmation interval 620 expires without receiving a Vsense signal from sensing circuit 86, control circuit 80 may determine the result of the GMA performed on the cardiac signal segment buffered during the pause confirmation interval 620. If the GMA result is VT/VF, control circuit 80 may restart the pause confirmation interval 620 without therapy delivery circuit 84 delivering a pacing pulse. In the example shown, however, the GMA result is not a VT/VF classification of the cardiac signal segment as indicated at 621 (e.g., the GMA result may be either a non-VT/VF or asystole classification). Therapy delivery circuit 84 may deliver a ventricular pacing pulse 622. Control circuit 80 may start a pacing period 640 and start a pacing escape interval 612 to schedule the next pacing pulse 634, e.g., according to a programmed post-shock pacing rate.

Buffering of cardiac signal segments for GMA may remain enabled during the pacing period 640. However, in order to avoid a very slow pacing rate or long delays between pacing pulses for buffering a relatively long cardiac signal segment for GMA, control circuit 80 may buffer the morphology signal 602 during multiple, relatively short time intervals 630a-630e during pacing escape intervals 612.

Control circuit 80 may wait for a pacing artifact delay interval 635 after the first pacing pulse 622 and each subsequent pacing pulse 634 before starting cardiac signal segment buffering during the relatively short time intervals 630a-630e. If a Vsense signal is not received during a pacing escape interval 612, therapy delivery circuit 84 may deliver a pacing pulse 634 upon expiration of the pacing escape interval 612. Control circuit 80 may restart the pacing escape interval 612, wait for a pacing artifact delay interval 635 and buffer a cardiac signal segment sensed over the relatively short time interval shown by each of time intervals 630 a-630e. The pacing artifact delay interval 635 may be 200 to 500 ms in various examples and may be the same or relatively shorter than the post-shock delay interval 605.

One relatively short cardiac signal segment corresponding to one of time intervals 630a-630e may be insufficient for determining a VT/VF, non-VT/VF or asystole result with a high degree of confidence based on the GMA, however. As such, control circuit 80 may determine VT/VF morphology metrics over multiple relatively short time segments 630a-630e for determining a GMA result from the cumulative time duration of the multiple post-pace cardiac signal segments. The VT/VF morphology metrics determined from each cardiac signal segment buffered over time segments 630a-630e may include the SW and MP and in some examples an amplitude metric, rate metric, LSC, and/or noise metric(s), e.g., as generally described above in conjunction with FIG. 9. The VT/VF morphology metrics determined for each individual post-pace cardiac signal segment corresponding to time intervals 630a-630e can be buffered as gross morphology data in memory 82. In some examples, control circuit 80 may determine if the relatively short cardiac signal segment is classified as VT/VF, asystole, or non-VT/VF based on the determined metrics and modified thresholds for classifying the relatively short cardiac signal segments. Control circuit 80 may label the cardiac signal segment in memory 82 accordingly.

Control circuit 80, however, may not delay, withhold or terminate the pacing period 640 or override a VT/VF rejection rule based on the GMA classification of one relatively short cardiac signal segment corresponding to one of time segments 630a-630e. A pending pacing pulse 634 may be delivered by therapy delivery circuit 84 upon expiration of the pacing escape interval 612 (when a Vsense signal is not received during the pacing escape interval) until a specified number of short cardiac signal segments are acquired and analyzed according to the GMA. Control circuit 80 may be configured to accumulate gross morphology data from multiple post-pace cardiac signal segments sensed over time intervals 630a-630e during successive pacing escape intervals 612, each started in response to a delivered pacing pulse (VP) 632 or 634. Control circuit 80 may determine the GMA result of the accumulated post-pace cardiac signal segments buffered over time intervals 630a-630e as being VT/VF when a threshold number of the post-pace cardiac signal segments (corresponding to time segments 630a-630e, collectively 630) are individually classified as being VT/VF evidence segments.

An individual post-pace cardiac signal segment may be classified as VT/VF based on modified VT/VF criteria compared to the VT/VF criteria that may be applied to a relatively longer cardiac signal segment, e.g., a 3-second segment that may be buffered during pause confirmation interval 620. For example, as described in conjunction with FIG. 9, when time intervals 630a-630e are 0.5 seconds, control circuit 80 may classify the corresponding relatively short cardiac signal segments as VT/VF when the MP is at least 60 and the SW/MP ratio is greater than −1 and less than or equal to 0.085. In an example, when at least 3, 4, 5, 6 or other specified number of relatively short cardiac signal segments are classified as VT/VF, e.g., according to modified VT/VF criteria, the GMA result may be determined to be VT/VF by control circuit 80. The threshold number of short cardiac signal segments classified as VT/VF may or may not be required to be consecutive during the pacing period 640.

In the illustrative example shown, control circuit 80 may classify the short cardiac signal segment corresponding to time interval 630e as VT/VF. When the VT/VF classification is the 5^{th} VT/VF classification (e.g., all of cardiac signal segments corresponding to time intervals 630a-e are classified as VT/VF), control circuit 80 determines a GMA result as being VT/VF as indicated at time 636. Control circuit 80 may cancel the pending pacing pulse scheduled at the expiration of the currently running pacing escape interval 612 in response to the GMA result of VT/VF based on the VT/VF classification of the threshold number of short cardiac signal segments. Control circuit 80 may terminate the pacing period 640 in response to the GMA result of VT/VF.

Control circuit 80 may restart the pause confirmation interval 620′. In this way, a GMA result may be obtained based on the classifications of multiple discontinuous cardiac signal segments during post-shock pacing for use in terminating a pacing period and for VT/VF redetection. The multiple discontinuous cardiac signal segments may be two or more segments and may or may not have the same time duration. When combined, however, the multiple discontinuous cardiac signal segments have a cumulative time duration that can provide a GMA result of VT/VF, non-VT/VF or asystole based on the classifications of the individual cardiac signal segments. It is to be understood that the threshold number of short cardiac signal segments for determining a GMA result of VT/VF may be a percentage of short cardiac signal segments (e.g., X out of Y). The short cardiac signal segments being classified as VT/VF may not necessarily be required to be consecutive as shown in FIG. 14. When a required number of short signal segments having a minimum cumulative cardiac signal segment time duration have been analyzed, a GMA result can be obtained. For example, at least Y short cardiac signal segments or a total minimum cumulative time duration of 2, 2.5, 3, or 3.5 seconds or other specified cumulative time duration of multiple cardiac signal segments may be analyzed for obtaining the GMA result.

When less than the threshold number of short cardiac signal segments are classified as VT/VF (e.g., less than X of Y), the GMA result may be non-VT/VF or asystole. More generally, when less than a specified percentage of the cumulative time duration of multiple cardiac signal segments is classified as VT/VF, the GMA result is not VT/VF (e.g., asystole or non-VT/VF). If asystole criteria are met for a threshold number of the short cardiac signal segments or percentage of the cumulative time duration of multiple cardiac signal segments, the GMA result may be asystole. In response to non-VT/VF or asystole GMA results, the pacing period 640 may continue running. As long as less than a threshold number of the short cardiac signal segments (or less than a percentage of the cumulative time duration of the cardiac signal segments) are classified as VT/VF (and a tachyarrhythmia operating state transition does not occur), pacing period 640 may continue running.

In various examples, the GMA result of VT/VF for terminating the pacing period 640 may require that at least a minimum number of the short cardiac signal segments, e.g., 2, 3 4, 5, 6, 7, or 8 cardiac signal segments, buffered during pacing escape intervals be classified as VT/VF before terminating the pacing period 640. For example, assuming no Vsense signals are received, at least 3, 4, 5, 6, 8 or other selected number of pacing pulses may be delivered before control circuit 80 terminates the pacing period 640 based on the GMA of the relatively short cardiac signal segments sensed between delivered pacing pulses. Control circuit 80 may determine the GMA result is a VT/VF classification when a predetermined percentage or ratio, e.g., at least half, at least two-thirds, at least three-fourths, 100% or other selected percentage or portion of a specified minimum number of cardiac signal segments buffered during pacing escape intervals 612 are classified as VT/VF segments. In some examples, the most recent one, two, or three cardiac signal segments may be required to be classified as VT/VF in addition to the threshold number of short cardiac signal segments being classified as being VT/VF in order to terminate the pacing period 640 based on the GMA result.

The GMA result based on multiple relatively short cardiac signal segments obtained during post-shock pacing may be used for overriding a rejection rule that has been met. When the GMA is not VT/VF, a rejection rule that is met may cause control circuit 80 to withhold VT/VF redetection if the RNID is reached. However, if the GMA result based on multiple post-pace cardiac signal segments is VT/VF, a rejection rule may be overridden such that control circuit 80 may redetect VT/VF if the RNID is reached.

In other examples, the post-pace cardiac signal segments obtained during multiple pacing intervals may be obtained for up to a specified number of delivered pacing pulses. After the specified number of pacing pulses, e.g., 3 to 15 pacing pulses, control circuit 80 may control the therapy delivery circuit 84 to withhold the next pacing pulse for at least three seconds (or other specified time interval) to allow buffering of one full length cardiac signal segment that is normally used for performing GMA when pacing is not being delivered, e.g., a 3-second cardiac signal segment. If the GMA is not VT/VF, control circuit 80 may enable therapy delivery circuit 84 to deliver a pacing pulse and resume delivery of ventricular pacing pulses at the post-shock pacing rate for another set of the specified number of pacing pulses or until the pacing period 640 expires. Control circuit 80 may resume buffering post-pace cardiac signal segments during delivery of another specified number of pacing pulses.

In still other examples, the relatively short, post-pace cardiac signal segments may be classified according to the GMA (with modified thresholds applied to morphology metrics as needed). When a threshold number of VT/VF classifications of the relatively short, post-pace cardiac signals segments (obtained during pacing escape intervals) are determined, which may be one or more, control circuit 80 may control therapy delivery circuit 84 to withhold the next scheduled pacing pulse. Control circuit 80 may start a pause confirmation interval to allow a full length, continuous cardiac signal segment to be obtained, e.g., a 3-second cardiac signal segment, for obtaining a GMA result based on one relatively long, continuous cardiac signal segment. This GMA result can be used for terminating the pacing period 640 and/or redetecting VT/VF. In this way, the GMA classifications of the relatively short, discontinuous post-pace cardiac signal segments obtained during pacing escape intervals can be used to trigger the buffering of a relatively longer, continuous cardiac signal segment during pacing inhibition. The relatively longer, continuous cardiac signal segment can be analyzed for obtaining the GMA result used for overriding a rejection rule and/or terminating the post-shock pacing period.

In the example of FIG. 14, no Vsense signals are received during the post-shock pacing period 640. It is recognized that in some instances a Vsense signal may be received during a pacing escape interval. In this case, the pacing escape interval may be restarted and the post-pace cardiac signal segment (if started) may be discarded. The next post-pace cardiac signal segment can be buffered after a subsequent pacing pulse is delivered. However, in other examples, if a Vsense signal is received before a pacing escape interval expires, buffering of a post-pace cardiac signal segment may continue when the pacing escape interval is restarted. Buffering of the post-pace cardiac signal may continue until a pacing escape interval expires or until a cardiac signal segment has been buffered for the normal time duration, e.g., 3 seconds, whichever comes first. If a full 3-second cardiac signal segment is buffered before the next pacing pulse is delivered, for example, the GMA result may be determined according to classification criteria applied to the normal duration cardiac signal segment.

If less than a full 3-second cardiac signal segment is buffered, the partial cardiac signal segment could be discarded. Alternatively, if less than the full 3-second cardiac signal segment is buffered, the partial cardiac signal segment may be divided into multiple short cardiac signal segments, e.g., equal to the post-pace time intervals 630a-630e, (with any excess portion of the partial cardiac signal segment being discarded). For example, if 2.25 seconds of a cardiac signal segment is buffered due to a Vsense signal causing pacing inhibition followed by a subsequently delivered pacing pulse, four 0.5 second segments may be analyzed and the remaining 0.25 seconds may be discarded. Each short cardiac signal segment could be classified according to the GMA toward obtaining a GMA result after at least Y short cardiac signal segments or a minimum cumulative time duration of multiple cardiac signal segments are classified. As such, two more cardiac signal segments, which may have different durations, may be combined to obtain a cumulative time duration of cardiac signal segments from which a GMA result can be obtained.

Further disclosed herein is the subject matter of the following examples:

Example 1. A medical device that includes a sensing circuit configured to sense a plurality of cardiac signals and sense ventricular event signals from the plurality of cardiac signals. The medical device further including a control circuit in communication with the sensing circuit. The control circuit can be configured to, for each of a plurality of ventricular event signals sensed by the sensing circuit, determine sensed event data from the plurality of cardiac signals and obtain a first cardiac signal segment from the plurality of cardiac signals sensed by the sensing circuit. The first cardiac signal segment may have a first time duration and a starting time that is independent of a timing of a ventricular event signal sensed by the sensing circuit. The control circuit may be further configured to perform a morphology analysis of the first cardiac signal segment for classifying the first cardiac signal segment as being one of ventricular tachyarrhythmia, non-ventricular tachyarrhythmia or asystole. The control circuit may be further configured to determine, from the ventricular event signals sensed by the sensing circuit, that a first required number of tachyarrhythmia intervals is reached for detecting ventricular tachyarrhythmia, determine that a rejection rule for withholding a tachyarrhythmia detection is met based on the sensed event data and determine that the first cardiac signal segment is classified as ventricular tachyarrhythmia based on the morphology analysis. The control circuit may be further configured to override the rejection rule for withholding a tachyarrhythmia detection by detecting ventricular tachyarrhythmia in response to the first required number of tachyarrhythmia intervals being reached and the first cardiac signal segment being classified as ventricular tachyarrhythmia based on the morphology analysis. The medical device may further include a therapy delivery circuit including a capacitor. The therapy delivery circuit can be configured to start charging the capacitor for delivering a therapy in response to the control circuit detecting the ventricular tachy arrhythmia.

Example 2. The medical device of example 1 wherein the therapy delivery circuit is configured to deliver the therapy by delivering one of anti-tachycardia pacing or a CV/DF shock in response to the control circuit detecting the ventricular tachyarrhythmia. The control circuit can be further configured to obtain a post-therapy cardiac signal segment from one of the plurality of cardiac signals sensed by the sensing circuit after the therapy is delivered and determine from the sensed ventricular event signals that a second required number of tachyarrhythmia intervals is reached for redetecting the ventricular tachyarrhythmia after the therapy is delivered. The second required number of tachyarrhythmia intervals can be less than the first required number of tachyarrhythmia intervals. The control circuit may be further configured to determine that a rejection rule for withholding detection of the ventricular tachyarrhythmia is met based on an analysis of the plurality of cardiac signals sensed by the sensing circuit, determine that the post-therapy cardiac signal segment is not classified as ventricular tachyarrhythmia based on the morphology analysis, and, in response to the post-therapy cardiac signal segment not being classified as ventricular tachyarrhythmia, withhold redetection of the ventricular tachyarrhythmia in response to the second required number of tachyarrhythmia intervals being reached and the rejection rule being met.

Example 3. The medical device of example 1 wherein the therapy delivery circuit is configured to deliver the therapy by delivering one of anti-tachycardia pacing or a CV/DF shock in response to the control circuit detecting the ventricular tachyarrhythmia. The control circuit being further configured to obtain a post-therapy cardiac signal segment from one of the plurality of cardiac signals sensed by the sensing circuit after the therapy is delivered and determine from the sensed ventricular event signals that a second required number of tachyarrhythmia intervals is reached for redetecting the ventricular tachyarrhythmia after the therapy is delivered. The second required number of tachyarrhythmia intervals can be less than the first required number of tachyarrhythmia intervals. The control circuit may be further configured to determine that a rejection rule for withholding detection of the tachyarrhythmia is met based on an analysis of the plurality of cardiac signals, determine that the post-therapy cardiac signal segment is classified as ventricular tachyarrhythmia based on the morphology analysis and, in response to the classification of the post-therapy cardiac signal segment being ventricular tachyarrhythmia, override the rejection rule for withholding detection of the tachyarrhythmia. The control circuit may be configured to redetect the ventricular tachyarrhythmia in response to the second required number of tachyarrhythmia intervals being reached and the rejection rule being overridden.

Example 4. The medical device of any of examples 1-3 wherein determining that a rejection rule for withholding detection of the ventricular tachyarrhythmia is met can include, for each sensed ventricular event signal of the plurality of the sensed ventricular event signals, obtaining a sensed event signal segment obtained from the plurality of cardiac signals and determining at least a portion of the sensed event data from the sensed event signal segment. The sensed event signal segment may have a starting time dependent on a timing of the sensed ventricular event signal.

Example 5. The medical device of any of examples 1-4 wherein the control circuit can be further configured to determine the sensed event data by determining at least one or more of: a sensed event peak amplitude, an RR interval ending with the sensed event signal, and/or an R-wave morphology matching score.

Example 6. The medical device of example 5 wherein the control circuit is further configured to determine that a rejection rule for withholding detection of the ventricular tachyarrhythmia is met by increasing a cardiac event oversensing count in response to detecting an alternating pattern of a high sensed event peak amplitude and a low sensed event peak amplitude from the sensed event data and determining that an R-wave morphology matching score of the sensed event data that corresponds to at least one of the high sensed event peak amplitudes of the sensed event data meets a match threshold. The control circuit can be further configured to determine that the cardiac event oversensing count reaches an oversensing threshold value and determine that the rejection rule is met in response to determining that the cardiac event oversensing count reaches the oversensing threshold value.

Example 7. The medical device of example 5 wherein the control circuit may be further configured to determine that a rejection rule for withholding detection of the ventricular tachyarrhythmia is met by determining a most common RR interval range from the determined RR intervals, determining that less than a threshold percentage of the determined RR intervals match the most common RR interval range, and determining that at least a threshold number of the R-wave morphology matching scores are greater than a supraventricular tachyarrhythmia matching threshold. The control circuit may be further configured to determine that the rejection rule is met in response to determining that less than the threshold percentage of the determined RR intervals match the most common RR interval range and determining that at least the threshold number of the R-wave morphology matching scores are greater than the supraventricular tachyarrhythmia matching threshold.

Example 8. The medical device of example 7 wherein the control circuit is further configured to disable the rejection rule in response to the therapy delivery circuit delivering the therapy.

Example 9. The medical device of example 1 wherein the therapy delivery circuit may be configured to deliver the therapy by delivering one of anti-tachyarrhythmia pacing or a CV/DF shock in response to the control circuit detecting the ventricular tachyarrhythmia. The control circuit may be further configured to start a pause confirmation interval after the therapy is delivered, determine that a ventricular event signal is not sensed by the sensing circuit during the pause confirmation interval, and obtain a post-therapy cardiac signal segment during the pause confirmation interval. The control circuit may be further configured to determine that the post-therapy cardiac signal segment is classified as one of asystole or non-ventricular tachyarrhythmia based on the morphology analysis and start a pacing period in response to the post-therapy cardiac signal segment being classified as one of asystole or non-ventricular tachyarrhythmia based on the morphology analysis.

Example 10. The medical device of example 9 wherein the therapy delivery circuit is further configured to deliver cardiac pacing pulses in response to the control circuit starting the pacing period. The control circuit may be further configured to obtain at least one post-pace cardiac signal segment and determine that at least a threshold portion of a cumulative time duration of a plurality of cardiac signal segments comprising the at least one post-pace cardiac signal segment is classified as ventricular tachyarrhythmia based on the morphology analysis. The control circuit may be further configured to redetect the ventricular tachyarrhythmia in response to at least the threshold portion of the cumulative time duration of the plurality of cardiac signal segments being classified as ventricular tachyarrhythmia. The control circuit may be further configured to terminate the pacing period in response to redetecting the ventricular tachyarrhythmia.

Example 11. The medical device of example 10 wherein the control circuit may be further configured to schedule each of a plurality of cardiac pacing pulses delivered by the therapy delivery circuit during the pacing period by starting a pacing escape interval. The control circuit may be further configured to obtain the cumulative time duration of the plurality of cardiac signal segments by obtaining each of a plurality of post-pace cardiac signal segments during a respective one of the pacing escape intervals.

Example 12. The medical device of example 9 wherein the therapy delivery circuit can be further configured to deliver cardiac pacing pulses in response to the control circuit starting the pacing period. The control circuit may be further configured to obtain one or more post-pace cardiac signal segments where each post-pace cardiac signal segment can be shorter than the first time duration of the first cardiac signal segment. The control circuit may be further configured to determine that at least a threshold portion of the one or more post-pace cardiac signal segments are classified as ventricular tachyarrhythmia based on the morphology analysis. The control circuit may be further configured to control the therapy delivery circuit to withhold delivery of cardiac pacing in response to at least the threshold portion of the one or more post-pace cardiac signal segments being classified as ventricular tachyarrhythmia. The control circuit may obtain a second cardiac signal segment while the cardiac pacing is being withheld. The second cardiac signal segment may have a second time duration equal to the first time duration of the first cardiac signal segment. The control circuit may be further configured to determine if the second cardiac signal segment is classified as ventricular tachyarrhythmia based on the morphology analysis. The control circuit may be configured to terminate the pacing period when the second cardiac signal segment is classified as ventricular tachyarrhythmia based on the morphology analysis. The control circuit may be further configured to control the therapy delivery circuit to resume delivering cardiac pacing pulses in response to the second cardiac signal segment not being classified as ventricular tachyarrhythmia based on the morphology analysis.

Example 13. The medical device of any of examples 1-12 wherein the control circuit is further configured to perform the morphology analysis of the first cardiac signal segment by determining an amplitude metric from the first cardiac signal segment, performing an asystole analysis for classifying the first cardiac signal segment as one of asystole or non-ventricular tachyarrhythmia when the amplitude metric is less than an amplitude threshold, and performing a tachyarrhythmia analysis for classifying the first cardiac signal segment as one of ventricular tachyarrhythmia or non-ventricular tachyarrhythmia when the amplitude metric is at least the threshold amplitude.

Example 14. The medical device of example 13 wherein the control circuit is further configured to perform the tachyarrhythmia analysis of the first cardiac signal segment by determining at least one of a mean period from the first cardiac signal segment and/or a spectral width from the first cardiac signal segment.

Example 15. The medical device of any of examples 1-14 wherein the control circuit is further configured to obtain a previous cardiac signal segment from the plurality of cardiac signal segments prior to obtaining the first cardiac signal segment and determine that the previous cardiac signal segment is classified as one of non-ventricular tachyarrhythmia or asystole based on the morphology analysis performed on the previous cardiac signal segment. The control circuit may be configured to withhold detecting a ventricular tachyarrhythmia in response to the first required number of tachyarrhythmia intervals being reached, the previous cardiac signal segment being classified as one of non-ventricular tachyarrhythmia or asystole and the rejection rule being met.

Example 16. The medical device of example 1 wherein the control circuit is further configured to determine that abort therapy criteria are met based on an analysis of the plurality of cardiac signals sensed by the sensing circuit after starting charging of the capacitor. The therapy delivery circuit can be further configured to abort delivery of the therapy in response to the control circuit determining that the abort therapy criteria are met. The control circuit can be further configured to determine, from the ventricular sensed event signals, that a threshold number of tachyarrhythmia intervals is reached for redetecting the ventricular tachyarrhythmia after delivery of the therapy is aborted. The control circuit may be further configured to redetect the ventricular tachyarrhythmia in response to the threshold number of tachyarrhythmia intervals being reached and one of:
a) determining that the rejection rule for withholding a tachyarrhythmia detection is met;
or b) determining that the rejection rule for withholding a tachyarrhythmia detection is met and a second cardiac signal segment obtained from the plurality of cardiac signals is classified as ventricular tachyarrhythmia based on the morphology analysis.

Example 17. A method including sensing a plurality of cardiac signals, sensing ventricular event signals from the plurality of cardiac signals, and, for each of a plurality of sensed ventricular event signals, determining sensed event data from the plurality of cardiac signals. The method may further include obtaining a first cardiac signal segment from the plurality of cardiac signals. The first cardiac signal segment may have a first time duration and a starting time that is independent of a timing of a ventricular event signal sensed by the sensing circuit. The method may further include performing a morphology analysis of the first cardiac signal segment for classifying the first cardiac signal segment as being one of ventricular tachyarrhythmia, non-ventricular tachyarrhythmia or asystole. The method may further include determining from the ventricular event signals that a first required number of tachyarrhythmia intervals is reached for detecting ventricular tachyarrhythmia, determining that a rejection rule for withholding a tachyarrhythmia detection is met based on the sensed event data, and determining that the first cardiac signal segment is classified as ventricular tachyarrhythmia based on the morphology analysis. The method may further include overriding the rejection rule for withholding a tachyarrhythmia detection by detecting ventricular tachyarrhythmia in response to the first required number of tachyarrhythmia intervals being reached and the first cardiac signal segment being classified as ventricular tachyarrhythmia based on the morphology analysis. The method may further include starting charging a capacitor for delivering a therapy in response to detecting the ventricular tachyarrhythmia.

Example 18. The method of example 17 further comprising delivering the therapy by delivering one of anti-tachycardia pacing or a CV/DF shock in response to detecting the ventricular tachyarrhythmia and obtaining a post-therapy cardiac signal segment from one of the plurality of cardiac signals after the therapy is delivered. The method may further include determining from the sensed ventricular event signals that a second required number of tachyarrhythmia intervals is reached for redetecting the ventricular tachyarrhythmia after the therapy is delivered. The second required number of tachyarrhythmia intervals can be less than the first required number of tachyarrhythmia intervals. The method may further include determining that a rejection rule for withholding detection of the ventricular tachyarrhythmia is met based on an analysis of the plurality of cardiac signals and determining that the post-therapy cardiac signal segment is not classified as ventricular tachyarrhythmia based on the morphology analysis. The method may further include, in response to the post-therapy cardiac signal segment not being classified as ventricular tachyarrhythmia, withholding redetection of the ventricular tachyarrhythmia in response to the second required number of tachyarrhythmia intervals being reached and the rejection rule being met.

Example 19. The method of example 17 further comprising delivering the therapy by delivering one of anti-tachycardia pacing or a CV/DF shock in response to detecting the ventricular tachyarrhythmia and obtaining a post-therapy cardiac signal segment from one of the plurality of cardiac signals after the therapy is delivered. The method may further include determining from the sensed ventricular event signals that a second required number of tachyarrhythmia intervals is reached for redetecting the ventricular tachyarrhythmia after the therapy is delivered. The second required number of tachyarrhythmia intervals can be less than the first required number of tachyarrhythmia intervals. The method may further include determining that a rejection rule for withholding detection of the tachyarrhythmia is met based on an analysis of the plurality of cardiac signals, determining that the post-therapy cardiac signal segment is classified as ventricular tachyarrhythmia based on the morphology analysis, and, in response to the post-therapy cardiac signal segment being classified as ventricular tachyarrhythmia, overriding the rejection rule for withholding detection of the tachyarrhythmia. The method may further include redetecting the ventricular tachyarrhythmia in response to the second required number of tachyarrhythmia intervals being reached and the rejection rule being overridden.

Example 20. The method of any of examples 17-19 wherein determining that a rejection rule for withholding detection of the ventricular tachyarrhythmia is met comprises, for each sensed ventricular event signal of the plurality of the sensed ventricular event signals, obtaining a sensed event signal segment from the plurality of cardiac signals and determining at least a portion of the sensed event data from the sensed event signal segment. The sensed event signal segment can have a starting time dependent on a timing of the sensed ventricular event signal.

Example 21. The method of any of examples 17-20 wherein determining the sensed event data may include determining at least one of: a sensed event peak amplitude, an RR interval ending with the sensed event signal, and/or an R-wave morphology matching score.

Example 22. The method of example 21 wherein determining that a rejection rule for withholding detection of the ventricular tachyarrhythmia is met can include increasing a cardiac event oversensing count in response to detecting an alternating pattern of a high sensed event peak amplitude and a low sensed event peak amplitude from the sensed event data and determining that an R-wave morphology matching score of the sensed event data that corresponds to at least one of the high sensed event peak amplitudes of the sensed event data meets a match threshold. The method may further include determining that the cardiac event oversensing count reaches an oversensing threshold value and determining that the rejection rule is met in response to determining that the cardiac event oversensing count reaches the oversensing threshold value.

Example 23. The method of example 21 wherein determining that a rejection rule for withholding detection of the ventricular tachyarrhythmia is met may include determining a most common RR interval range from the determined RR intervals and determining that less than a threshold percentage of the determined RR intervals match the most common RR interval range. The method may further include determining that at least a threshold number of the R-wave morphology matching scores are greater than a supraventricular tachyarrhythmia matching threshold and determining that the rejection rule is met in response to determining that less than the threshold percentage of the determined RR intervals match the most common RR interval range and determining that at least the threshold number of the R-wave morphology matching scores are greater than the supraventricular tachyarrhythmia matching threshold.

Example 24. The medical device of example 23 further comprising disabling the rejection rule in response to the therapy delivery circuit delivering a therapy.

Example 25. The method of example 17 further comprising delivering the therapy by delivering one of anti-tachyarrhythmia pacing or a CV/DF shock in response to the control circuit detecting the ventricular tachyarrhythmia. The method may further include starting a pause confirmation interval after the therapy is delivered, determining that a ventricular event signal is not sensed during the pause confirmation interval, obtaining a post-therapy cardiac signal segment during the pause confirmation interval, and determining that the post-therapy cardiac signal segment is classified as one of asystole or non-ventricular tachyarrhythmia based on the morphology analysis. The method may further include starting a pacing period in response to the post-therapy cardiac signal segment being classified as one of asystole or non-ventricular tachyarrhythmia based on the morphology analysis.

Example 26. The method of example 25 further comprising delivering cardiac pacing pulses in response to starting the pacing period. The method may further include obtaining at least one post-pace cardiac signal segment and determining that at least a threshold portion of a cumulative time duration of a plurality of cardiac signal segments comprising the at least one post-pace cardiac signal segment is classified as ventricular tachyarrhythmia based on the morphology analysis. The method may further include redetecting the ventricular tachyarrhythmia in response to at least the threshold portion of the cumulative time duration of the plurality of cardiac signal segments being classified as ventricular tachyarrhythmia. The method may further include terminating the pacing period in response to redetecting the ventricular tachyarrhythmia.

Example 27. The method of example 26 further comprising scheduling each of a plurality of cardiac pacing pulses during the pacing period by starting a pacing escape interval and obtaining the cumulative time duration of the plurality of cardiac signal segments by obtaining each of a plurality of post-pace cardiac signal segments during a respective one of the pacing escape intervals.

Example 28. The method of example 25 further including delivering cardiac pacing pulses in response to starting the pacing period and obtaining one or more post-pace cardiac signal segments. Each post-pace cardiac signal segment can be shorter than the first time duration of the first cardiac signal segment. The method may further include determining that at least a threshold portion of the one or more post-pace cardiac signal segments are classified as ventricular tachyarrhythmia based on the morphology analysis. The method may further include, in response to at least the threshold portion of the one or more post-pace cardiac signal segments being classified as ventricular tachyarrhythmia, withholding delivery of cardiac pacing. The method may further include obtaining a second cardiac signal segment while cardiac pacing is being withheld. The second cardiac signal segment can have a second time duration equal to the first time duration of the first cardiac signal segment. The method may further include determining if the second cardiac signal segment is classified as ventricular tachyarrhythmia based on the morphology analysis. The method may further include terminating the pacing period when the second cardiac signal segment is classified as ventricular tachyarrhythmia based on the morphology analysis. The method may further include controlling the therapy delivery circuit to resume delivering cardiac pacing pulses in response to the second cardiac signal segment not being classified as ventricular tachyarrhythmia based on the morphology analysis.

Example 29. The method of any of examples 17-28 wherein performing the morphology analysis of the first cardiac signal segment may include determining an amplitude metric from the first cardiac signal segment, performing an asystole analysis for classifying the first cardiac signal segment as one of asystole or non-ventricular tachyarrhythmia when the amplitude metric is less than an amplitude threshold, and performing a tachyarrhythmia analysis for classifying the first cardiac signal segment as one of ventricular tachyarrhythmia or non-ventricular tachyarrhythmia when the amplitude metric is at least the threshold amplitude.

Example 30. The method of example 29 wherein performing the tachyarrhythmia analysis of the first cardiac signal segment may include determining at least one of a mean period from the first cardiac signal segment and/or determining a spectral width from the first cardiac signal segment.

Example 31. The method of any of examples 17-30 further including obtaining a previous cardiac signal segment from the plurality of cardiac signal segments prior to obtaining the first cardiac signal segment and determining that the previous cardiac signal segment is classified as one of non-ventricular tachyarrhythmia or asystole based on the morphology analysis performed on the previous cardiac signal segment. The method may further include withholding detecting a ventricular tachyarrhythmia in response to the first required number of tachyarrhythmia intervals being reached, the previous cardiac signal segment being classified as one of non-ventricular tachyarrhythmia or asystole and the rejection rule being met.

Example 32. The method of example 17 further including determining that abort therapy criteria are met based on an analysis of the plurality of cardiac signals sensed after starting charging of the capacitor. The method may further include aborting delivery of the therapy in response to determining that the abort therapy criteria are met. The method may further include determining from the sensed ventricular event signals that a threshold number of tachyarrhythmia intervals is reached for redetecting the ventricular tachyarrhythmia after the delivery of the therapy is aborted and redetecting the ventricular tachyarrhythmia in response to the threshold number of tachyarrhythmia intervals being reached and one of: a) determining that the rejection rule for withholding a tachyarrhythmia detection is not met; or b) determining that the rejection rule for withholding a tachyarrhythmia detection is met and a second cardiac signal segment obtained from the plurality of cardiac signals is classified as ventricular tachyarrhythmia based on the morphology analysis.

Example 33. A non-transitory, computer readable medium storing a set of instructions that, when executed by a control circuit of a medical device, cause the medical device to sense a plurality of cardiac signals, sense ventricular event signals from the plurality of cardiac signals and, for each of a plurality of sensed ventricular event signals, determine sensed event data from the plurality of cardiac signals. The instructions may further cause the medical device to obtain a cardiac signal segment from the plurality of cardiac signals. The cardiac signal segment can have a starting time that is independent of a timing of a ventricular event signal sensed by the sensing circuit. The instructions may further cause the medical device to perform a morphology analysis of the cardiac signal segment for classifying the cardiac signal segment as being one of ventricular tachyarrhythmia, non-ventricular tachyarrhythmia or asystole. The instructions may further cause the medical device to determine from the ventricular event signals that a required number of tachyarrhythmia intervals is reached for detecting ventricular tachyarrhythmia, determine that a rejection rule for withholding a tachyarrhythmia detection is met based on the sensed event data and determine that the cardiac signal segment is classified as ventricular tachyarrhythmia based on the morphology analysis. The instructions may further cause the medical device to override the rejection rule for withholding a tachyarrhythmia detection by detecting ventricular tachyarrhythmia in response to the required number of tachyarrhythmia intervals being reached and the first cardiac signal segment being classified as ventricular tachyarrhythmia based on the morphology analysis. The instructions may further cause the medical device to start charging a capacitor for delivering a therapy in response to detecting the ventricular tachyarrhythmia.

Example 34. A medical device including a sensing circuit configured to sense a plurality of cardiac signals and sense ventricular event signals from at least one of the plurality of cardiac signals. The medical device further includes a control circuit in communication with the sensing circuit. The control circuit can be configured to detect a ventricular tachyarrhythmia based on the sensed plurality of cardiac signals and, subsequent to detecting the ventricular tachyarrhythmia, determine from the ventricular event signals sensed by the sensing circuit that a redetection number of tachyarrhythmia intervals is reached. The control circuit can be further configured to determine when a first rejection rule for withholding a tachyarrhythmia detection is met based on an analysis of the plurality of cardiac signals. The control circuit may be further configured to withhold redetection of a ventricular tachyarrhythmia when the first rejection rule is met and the redetection number of tachyarrhythmia intervals is reached or redetect the ventricular tachyarrhythmia when the first rejection rule is not met and the redetection number of tachyarrhythmia intervals is reached.

Example 35. The medical device of example 34 wherein the control circuit may be further configured to determine when the first rejection rule for withholding a tachyarrhythmia detection is met by, for each of a plurality of the sensed ventricular event signals, obtaining a sensed event signal segment from the plurality of cardiac signals and determining sensed event data from the sensed event signal segment. The control circuit may be further configured to determine that the first rejection rule is met based on the sensed event data.

Example 36. The medical device of any of examples 34-35 wherein the control circuit is further configured to determine when the first rejection rule is met by determining when a supraventricular tachyarrhythmia rejection rule is met.

Example 37. The medical device of any of examples 34-35 wherein the control circuit is further configured to determine when the first rejection rule is met by determining a cardiac event oversensing rejection rule is met.

Example 38. The medical device of any of examples 34-35 wherein the control circuit is further configured to determine when the first rejection rule is met by determining when a noise rejection rule is met.

Example 39. The medical device of any of examples 34-38 further comprising a therapy delivery circuit configured to deliver a therapy. The therapy delivery circuit can be configured to deliver the therapy in response to the control circuit detecting the ventricular tachyarrhythmia. The control circuit can be further configured to determine from the ventricular event signals sensed by the sensing circuit that the redetection number of tachyarrhythmia intervals is reached after the therapy is delivered.

Example 40. The medical device of example 39 wherein the therapy delivery circuit is configured to deliver the therapy by delivering at least one of anti-tachycardia pacing or a cardioversion/defibrillation shock pulse.

Example 41. The medical device of any of examples 34-38 further comprising a therapy delivery circuit configured to deliver a therapy. The control circuit may be further configured to, subsequent to detecting the ventricular tachyarrhythmia, determine that abort therapy criteria are met based on the sensed plurality of cardiac signals. The control circuit may control the therapy delivery circuit to abort the therapy in response to the abort therapy criteria being met. The control circuit may be further configured to determine from the ventricular event signals sensed by the sensing circuit that the redetection number of tachyarrhythmia intervals is reached after the therapy is aborted.

Example 42. The medical device of any of examples 34-41 wherein the control circuit is further configured to detect the ventricular tachyarrhythmia by applying a first rejection rule for withholding a tachyarrhythmia detection, applying a second rejection rule for withholding a tachyarrhythmia detection, and detecting the ventricular tachyarrhythmia by determining that the first rejection rule is not met and the second rejection rule is not met. The control circuit may be further configured to disable the second rejection rule after detecting the ventricular tachyarrhythmia. The control circuit may be further configured to apply the first rejection rule after detecting the ventricular tachyarrhythmia and in response to determining that the redetection number of tachyarrhythmia interval is reached.

Example 43. The medical device of any of examples 34-42 wherein the control circuit is further configured to obtain a cardiac signal segment from the plurality of cardiac signal segments, classify the cardiac signal segment as ventricular tachyarrhythmia based on a morphology analysis of the cardiac signal segment, and override withholding redetection of the ventricular tachyarrhythmia by redetecting the ventricular tachyarrhythmia in response to the cardiac signal segment being classified as ventricular tachyarrhythmia and the redetection number of tachyarrhythmia intervals being reached.

Example 44. A method including sensing a plurality of cardiac signals, sensing ventricular event signals from at least one of the plurality of cardiac signals and detecting a ventricular tachyarrhythmia based on the sensed plurality of cardiac signals. Subsequent to detecting the ventricular tachyarrhythmia, the method may further include determining from the sensed ventricular event signals that a redetection number of tachy arrhythmia intervals is reached, determining when a first rejection rule for withholding a tachyarrhythmia detection is met based on an analysis of the plurality of cardiac signals. The method may further include withholding redetection of a ventricular tachyarrhythmia when the first rejection rule is met and the redetection number of tachyarrhythmia intervals is reached or redetecting the ventricular tachyarrhythmia when the first rejection rule is not met and the redetection number of tachyarrhythmia intervals is reached.

Example 45. The method of example 44 wherein determining when the first rejection rule for withholding a tachyarrhythmia detection is met can include, for each of a plurality of the sensed ventricular event signals, obtaining a sensed event signal segment from the plurality of cardiac signals and determining sensed event data from the sensed event signal segment. The method may further include determining that the first rejection rule is met based on the sensed event data.

Example 46. The method of any of examples 44-45 further comprising determining when the first rejection rule is met by determining when a supraventricular tachyarrhythmia rejection rule is met.

Example 47. The method of any of examples 44-45 further comprising determining when the first rejection rule is met by determining when a cardiac event oversensing rejection rule is met.

Example 48. The method of any of examples 44-45 further comprising determining when the first rejection rule is met by determining when a noise rejection rule is met.

Example 49. The method of any of examples 44-48 further including delivering a therapy in response to detecting the ventricular tachyarrhythmia and determining from the sensed ventricular event signals that the redetection number of tachyarrhythmia intervals is reached after the therapy is delivered.

Example 50. The method of example 49 wherein delivering the therapy comprises delivering at least one of anti-tachycardia pacing or a CV/DF shock pulse.

Example 51. The method of any of examples 44-48 further including, subsequent to detecting the ventricular tachyarrhythmia, determining that abort therapy criteria are met based on the sensed plurality of cardiac signals. The method may further include aborting a therapy in response to the abort therapy criteria being met. The method may further include determining from the sensed ventricular event signals that the redetection number of tachyarrhythmia intervals is reached after the therapy is aborted.

Example 52. The method of any of examples 44-51 further including detecting the ventricular tachyarrhythmia by applying the first rejection rule for withholding a tachyarrhythmia detection, applying a second rejection rule for withholding a tachyarrhythmia detection, and detecting the ventricular tachyarrhythmia by determining that the first rejection rule is not met and the second rejection rule is not met. The method may further include disabling the second rejection rule after detecting the ventricular tachyarrhythmia. The method may further include applying the first rejection rule after detecting the ventricular tachyarrhythmia and in response to determining that the redetection number of tachyarrhythmia interval is reached.

Example 53. The method of any of examples 44-52 further comprising obtaining a cardiac signal segment from the plurality of cardiac signal segments and classifying the cardiac signal segment as ventricular tachyarrhythmia based on a morphology analysis of the cardiac signal segment. The method may further include overriding withholding redetection of the ventricular tachyarrhythmia by redetecting the ventricular tachyarrhythmia in response to the cardiac signal segment being classified as ventricular tachyarrhythmia and the redetection number of tachyarrhythmia intervals being reached.

Example 54. A non-transitory, computer readable medium storing a set of instructions that, when executed by a control circuit of a medical device, cause the medical device to sense a plurality of cardiac signals, sense ventricular event signals from at least one of the plurality of cardiac signals, detect a ventricular tachyarrhythmia based on the sensed plurality of cardiac signals and, subsequent to detecting the ventricular tachyarrhythmia, determine from the sensed ventricular event signals that a redetection number of tachyarrhythmia intervals is reached. The instructions may further cause the medical device to determine when a first rejection rule for withholding a tachyarrhythmia detection is met based on an analysis of the plurality of cardiac signals and withhold redetection of a ventricular tachyarrhythmia when the first rejection rule is met and the redetection number of tachyarrhythmia intervals is reached or redetect the ventricular tachyarrhythmia when the first rejection rule is not met and the redetection number of tachyarrhythmia intervals is reached.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPLAs), or other equivalent integrated or discrete logic circuitry. Accordingly, the term “processor,” as used herein may refer to any of the foregoing structure or any other structure suitable for implementation of the techniques described herein. Also, the techniques could be fully implemented in one or more circuits or logic elements.

Thus, a medical device has been presented in the foregoing description with reference to specific examples. It is to be understood that various aspects disclosed herein may be combined in different combinations than the specific combinations presented in the accompanying drawings. It is appreciated that various modifications to the referenced examples may be made without departing from the scope of the disclosure and the following claims.

1. A medical device comprising:
a sensing circuit configured to:
sense a plurality of cardiac signals; and
sense ventricular event signals from the plurality of cardiac signals;
a control circuit in communication with the sensing circuit, the control circuit configured to:
for each of a plurality of ventricular event signals sensed by the sensing circuit, determine sensed event data from the plurality of cardiac signals;
obtain a first cardiac signal segment from the plurality of cardiac signals sensed by the sensing circuit, the first cardiac signal segment having a first time duration and a starting time that is independent of a timing of a ventricular event signal sensed by the sensing circuit;
perform a morphology analysis of the first cardiac signal segment for classifying the first cardiac signal segment as being one of ventricular tachyarrhythmia, non-ventricular tachyarrhythmia or asystole;
determine from the ventricular event signals sensed by the sensing circuit that a first required number of tachyarrhythmia intervals is reached for detecting ventricular tachyarrhythmia;
determine that a rejection rule for withholding a tachyarrhythmia detection is met based on the sensed event data;
determine that the first cardiac signal segment is classified as ventricular tachyarrhythmia based on the morphology analysis; and
override the rejection rule for withholding a tachyarrhythmia detection by detecting ventricular tachyarrhythmia in response to the first required number of tachyarrhythmia intervals being reached and the first cardiac signal segment being classified as ventricular tachyarrhythmia based on the morphology analysis; and
a therapy delivery circuit comprising a capacitor, the therapy delivery circuit configured to start charging the capacitor for delivering a therapy in response to the control circuit detecting the ventricular tachyarrhythmia.

2. The medical device of claim 1 wherein:
the therapy delivery circuit is configured to deliver the therapy by delivering one of anti-tachycardia pacing or a CV/DF shock in response to the control circuit detecting the ventricular tachyarrhythmia; and
the control circuit is further configured to:
obtain a post-therapy cardiac signal segment from one of the plurality of cardiac signals sensed by the sensing circuit after the therapy is delivered;
determine from the sensed ventricular event signals that a second required number of tachyarrhythmia intervals is reached for redetecting the ventricular tachyarrhythmia after the therapy is delivered, the second required number of tachyarrhythmia intervals being less than the first required number of tachyarrhythmia intervals;
determine that a rejection rule for withholding detection of the ventricular tachyarrhythmia is met based on an analysis of the plurality of cardiac signals sensed by the sensing circuit;
determine that the post-therapy cardiac signal segment is not classified as ventricular tachyarrhythmia based on the morphology analysis; and
in response to the post-therapy cardiac signal segment not being classified as ventricular tachyarrhythmia, withhold redetection of the ventricular tachyarrhythmia in response to the second required number of tachyarrhythmia intervals being reached and the rejection rule being met.

3. The medical device of claim 1 wherein:
the therapy delivery circuit is configured to deliver the therapy by delivering one of anti-tachycardia pacing or a CV/DF shock in response to the control circuit detecting the ventricular tachyarrhythmia;
the control circuit is further configured to:
obtain a post-therapy cardiac signal segment from one of the plurality of cardiac signals sensed by the sensing circuit after the therapy is delivered;
determine from the sensed ventricular event signals that a second required number of tachyarrhythmia intervals is reached for redetecting the ventricular tachyarrhythmia after the therapy is delivered, the second required number of tachyarrhythmia intervals being less than the first required number of tachyarrhythmia intervals;
determine that a rejection rule for withholding detection of the tachyarrhythmia is met based on an analysis of the plurality of cardiac signals;
determine that the post-therapy cardiac signal segment is classified as ventricular tachyarrhythmia based on the morphology analysis;
in response to the classification of the post-therapy cardiac signal segment being ventricular tachyarrhythmia, override the rejection rule for withholding detection of the tachyarrhythmia; and
redetect the ventricular tachyarrhythmia in response to the second required number of tachyarrhythmia intervals being reached and the rejection rule being overridden.

4. The medical device of claim 1 wherein determining that a rejection rule for withholding detection of the ventricular tachyarrhythmia is met comprises, for each sensed ventricular event signal of the plurality of the sensed ventricular event signals:
obtaining a sensed event signal segment obtained from the plurality of cardiac signals, the sensed event signal segment having a starting time dependent on a timing of the sensed ventricular event signal; and
determining at least a portion of the sensed event data from the sensed event signal segment.

5. The medical device of claim 1 wherein the control circuit is further configured to determine the sensed event data by determining:
a sensed event peak amplitude;
an RR interval ending with the sensed event signal; and
an R-wave morphology matching score.

6. The medical device of claim 5 wherein the control circuit is further configured to determine that a rejection rule for withholding detection of the ventricular tachyarrhythmia is met by:
increasing a cardiac event oversensing count in response to:
detecting an alternating pattern of a high sensed event peak amplitude and a low sensed event peak amplitude from the sensed event data; and
determining that an R-wave morphology matching score of the sensed event data that corresponds to at least one of the high sensed event peak amplitudes of the sensed event data meets a match threshold;
determining that the cardiac event oversensing count reaches an oversensing threshold value; and
determining that the rejection rule is met in response to determining that the cardiac event oversensing count reaches the oversensing threshold value.

7. The medical device of claim 5 wherein the control circuit is further configured to determine that a rejection rule for withholding detection of the ventricular tachyarrhythmia is met by:
determining a most common RR interval range from the determined RR intervals;
determining that less than a threshold percentage of the determined RR intervals match the most common RR interval range;
determining that at least a threshold number of the R-wave morphology matching scores are greater than a supraventricular tachyarrhythmia matching threshold; and
determining that the rejection rule is met in response to determining that less than the threshold percentage of the determined RR intervals match the most common RR interval range and determining that at least the threshold number of the R-wave morphology matching scores are greater than the supraventricular tachyarrhythmia matching threshold.

8. The medical device of claim 7 wherein the control circuit is further configured to disable the rejection rule in response to the therapy delivery circuit delivering the therapy.

9. The medical device of claim 1 wherein:
the therapy delivery circuit is configured to deliver the therapy by delivering one of anti-tachyarrhythmia pacing or a CV/DF shock in response to the control circuit detecting the ventricular tachyarrhythmia; and
the control circuit is further configured to:
start a pause confirmation interval after the therapy is delivered;
determine that a ventricular event signal is not sensed by the sensing circuit during the pause confirmation interval;
obtain a post-therapy cardiac signal segment during the pause confirmation interval;
determine that the post-therapy cardiac signal segment is classified as one of asystole or non-ventricular tachyarrhythmia based on the morphology analysis; and
start a pacing period in response to the post-therapy cardiac signal segment being classified as one of asystole or non-ventricular tachyarrhythmia based on the morphology analysis.

10. The medical device of claim 9 wherein:
the therapy delivery circuit is further configured to deliver cardiac pacing pulses in response to the control circuit starting the pacing period; and
the control circuit is further configured to:
obtain at least one post-pace cardiac signal segment;
determine that at least a threshold portion of a cumulative time duration of a plurality of cardiac signal segments comprising the at least one post-pace cardiac signal segment is classified as ventricular tachyarrhythmia based on the morphology analysis;
redetect the ventricular tachyarrhythmia in response to at least the threshold portion of the cumulative time duration of the plurality of cardiac signal segments being classified as ventricular tachyarrhythmia; and
terminate the pacing period in response to redetecting the ventricular tachyarrhythmia.

11. The medical device of claim 9 wherein:
the therapy delivery circuit is further configured to deliver cardiac pacing pulses in response to the control circuit starting the pacing period;
the control circuit is further configured to:
obtain one or more post-pace cardiac signal segments, each post-pace cardiac signal segment being shorter than the first time duration of the first cardiac signal segment;
determine that at least a threshold portion of the one or more post-pace cardiac signal segments are classified as ventricular tachyarrhythmia based on the morphology analysis;
control the therapy delivery circuit to withhold delivery of cardiac pacing in response to at least the threshold portion of the one or more post-pace cardiac signal segments being classified as ventricular tachyarrhythmia; and
obtain a second cardiac signal segment while cardiac pacing is being withheld, the second cardiac signal segment having a second time duration equal to the first time duration of the first cardiac signal segment;
determine if the second cardiac signal segment is classified as ventricular tachyarrhythmia based on the morphology analysis;
terminate the pacing period when the second cardiac signal segment is classified as ventricular tachyarrhythmia based on the morphology analysis; and
control the therapy delivery circuit to resume delivering cardiac pacing pulses in response to the second cardiac signal segment not being classified as ventricular tachyarrhythmia based on the morphology analysis.

12. The medical device of claim 1 wherein the control circuit is further configured to perform the morphology analysis of the first cardiac signal segment by:
determining an amplitude metric from the first cardiac signal segment;
performing an asystole analysis for classifying the first cardiac signal segment as one of asystole or non-ventricular tachyarrhythmia when the amplitude metric is less than an amplitude threshold; and
performing a tachyarrhythmia analysis for classifying the first cardiac signal segment as one of ventricular tachyarrhythmia or non-ventricular tachyarrhythmia when the amplitude metric is at least the threshold amplitude.

13. The medical device of claim 12 wherein the control circuit is further configured to perform the tachyarrhythmia analysis of the first cardiac signal segment by determining at least one of:
a mean period from the first cardiac signal segment; and
a spectral width from the first cardiac signal segment.

14. The medical device of claim 1 wherein the control circuit is further configured to:
obtain a previous cardiac signal segment from the plurality of cardiac signal segments prior to obtaining the first cardiac signal segment;
determine that the previous cardiac signal segment is classified as one of non-ventricular tachyarrhythmia or asystole based on the morphology analysis performed on the previous cardiac signal segment; and
withhold detecting a ventricular tachyarrhythmia in response to the first required number of tachyarrhythmia intervals being reached, the previous cardiac signal segment being classified as one of non-ventricular tachyarrhythmia or asystole and the rejection rule being met.

15. The medical device of claim 1 wherein:
the control circuit is further configured to determine that abort therapy criteria are met based on an analysis of the plurality of cardiac signals sensed by the sensing circuit after starting charging of the capacitor;
the therapy delivery circuit is further configured to abort delivery of the therapy in response to the control circuit determining that the abort therapy criteria are met;
the control circuit being further configured to:
determine from the ventricular sensed event signals that a threshold number of tachyarrhythmia intervals is reached for redetecting the ventricular tachyarrhythmia after delivery of the therapy is aborted; and
redetect the ventricular tachyarrhythmia in response to the threshold number of tachyarrhythmia intervals being reached and one of:
a) determining that the rejection rule for withholding a tachyarrhythmia detection is not met; or
b) determining that the rejection rule for withholding a tachyarrhythmia detection is met and a second cardiac signal segment obtained from the plurality of cardiac signals is classified as ventricular tachyarrhythmia based on the morphology analysis.

16. A method comprising:
sensing a plurality of cardiac signals;
sensing ventricular event signals from at least one of the plurality of cardiac signals;
detecting a ventricular tachyarrhythmia based on the sensed plurality of cardiac signals; and
subsequent to detecting the ventricular tachyarrhythmia:
determining from the sensed ventricular event signals that a redetection number of tachyarrhythmia intervals is reached;
determining when a first rejection rule for withholding a tachyarrhythmia detection is met based on an analysis of the plurality of cardiac signals; and
withholding redetection of a ventricular tachyarrhythmia when the first rejection rule is met and the redetection number of tachyarrhythmia intervals is reached; and
redetecting the ventricular tachyarrhythmia when the first rejection rule is not met and the redetection number of tachyarrhythmia intervals is reached.

17. The method of claim 16 further comprising:
delivering the therapy by delivering one of anti-tachycardia pacing or a CV/DF shock in response to detecting the ventricular tachyarrhythmia;
obtaining a post-therapy cardiac signal segment from one of the plurality of cardiac signals after the therapy is delivered;
determining from the sensed ventricular event signals that a second required number of tachyarrhythmia intervals is reached for redetecting the ventricular tachyarrhythmia after the therapy is delivered, the second required number of tachyarrhythmia intervals being less than the first required number of tachyarrhythmia intervals;
determining that a rejection rule for withholding detection of the tachyarrhythmia is met based on an analysis of the plurality of cardiac signals;
determining that the post-therapy cardiac signal segment is classified as ventricular tachyarrhythmia based on the morphology analysis;
in response to the post-therapy cardiac signal segment being classified as ventricular tachyarrhythmia, overriding the rejection rule for withholding detection of the tachyarrhythmia; and
redetecting the ventricular tachyarrhythmia in response to the second required number of tachyarrhythmia intervals being reached and the rejection rule being overridden.

18. The method of claim 16 wherein determining that a rejection rule for withholding detection of the ventricular tachyarrhythmia is met comprises, for each sensed ventricular event signal of the plurality of the sensed ventricular event signals:
obtaining a sensed event signal segment from the plurality of cardiac signals, the sensed event signal segment having a starting time dependent on a timing of the sensed ventricular event signal; and
determining at least a portion of the sensed event data from the sensed event signal segment.

19. The method of claim 16 wherein determining the sensed event data comprises determining:
a sensed event peak amplitude;
an RR interval ending with the sensed event signal; and
an R-wave morphology matching score.

20. The method of claim 16 further comprising:
delivering the therapy by delivering one of anti-tachyarrhythmia pacing or a CV/DF shock in response to the control circuit detecting the ventricular tachyarrhythmia;
starting a pause confirmation interval after the therapy is delivered;
determining that a ventricular event signal is not sensed during the pause confirmation interval;
obtaining a post-therapy cardiac signal segment during the pause confirmation interval;
determining that the post-therapy cardiac signal segment is classified as one of asystole or non-ventricular tachyarrhythmia based on the morphology analysis;
starting a pacing period in response to the post-therapy cardiac signal segment being classified as one of asystole or non-ventricular tachyarrhythmia based on the morphology analysis; and
delivering cardiac pacing pulses in response to starting the pacing period.

21. A non-transitory, computer readable medium storing a set of instructions that, when executed by a control circuit of a medical device, cause the medical device to:
sense a plurality of cardiac signals;
sense ventricular event signals from the plurality of cardiac signals;
for each of a plurality of sensed ventricular event signals, determine sensed event data from the plurality of cardiac signals;
obtain a cardiac signal segment from the plurality of cardiac signals, the cardiac signal segment having a starting time that is independent of a timing of a ventricular event signal sensed by the sensing circuit;
perform a morphology analysis of the cardiac signal segment for classifying the cardiac signal segment as being one of ventricular tachyarrhythmia, non-ventricular tachyarrhythmia or asystole;
determine from the ventricular event signals that a required number of tachyarrhythmia intervals is reached for detecting ventricular tachyarrhythmia;
determine that a rejection rule for withholding a tachyarrhythmia detection is met based on the sensed event data;
determine that the cardiac signal segment is classified as ventricular tachyarrhythmia based on the morphology analysis;
override the rejection rule for withholding a tachyarrhythmia detection by detecting ventricular tachyarrhythmia in response to the required number of tachyarrhythmia intervals being reached and the first cardiac signal segment being classified as ventricular tachyarrhythmia based on the morphology analysis; and
start charging a capacitor for delivering a therapy in response to detecting the ventricular tachyarrhythmia.
